(19) United States
(12) Patent Application Publication
(10) Pub. No.: US 2007/0207555 A1
(43) Pub. Date: Sep. 6, 2007

(54) Ultra-sensitive detection systems using multidimension signals

(76) Inventors: Cesar Guerra, Guilford, CT (US); Darin Latimer, East Haven, CT (US)

Correspondence Address: WILMER CUTLER PICKERING HALE AND DORR LLP, 60 STATE STREET, BOSTON, MA, 02109, US

(21) Appl. No.: 11/344,801

(22) Filed: Feb. 1, 2006

Related U.S. Application Data
(60) Provisional application No. 60/649,897, filed on Feb. 3, 2005.

(51) Int. Cl. G01N 33/543; G06F 19/00

(52) U.S. Cl. 436/518; 702/19

(57) ABSTRACT

Disclosed are compositions and methods for sensitive detection of one or multiple analytes. In general, the methods involve the use of special label components, referred to as multidimension signals. In the disclosed methods, analysis of multidimension signals can result in one or more predetermined patterns that serve to indicate whether a further level of analysis can or should be performed and/or which portion(s) of the analyzed material can or should be analyzed in a further level of analysis. In some forms, isobaric and non-isobaric elements can be used together in the same assay or assay system. Isobaric and non-isobaric multidimension signals used together can generate one or more predetermined patterns during analysis. The pattern generated in this first level of analysis indicates whether the second level of analysis should be performed. The second level of analysis can involve distinguishing the isobaric multidimension signals.

### CROSS-REFERENCE TO RELATED APPLICATIONS

This application claims priority to U.S. Provisional Application Ser. No. 60/649,897 filed Feb. 3, 2005, the entire contents of which are hereby incorporated by reference.

### FIELD OF THE INVENTION

### Background of the Invention

This invention is generally in the field of detection of analytes and biomolecules, and more specifically in the field of multiplex detection and analysis of analytes and biomolecules.

Detection of molecules is an important operation in the biological and medical sciences. Such detection often requires the use of specialized label molecules, amplification of a signal, or both, because many molecules of interest are present in low quantities and do not, by themselves, produce detectable signals. Many labels, labeling systems, and signal amplification techniques have been developed. For example, proteins have been detected using antibody-based detection systems such as sandwich assays (Mailini and Maysef, “A sandwich method for enzyme immunoassay. I. Application to rat and human alpha-fetoprotein” J. Immunol. Methods 8:223-234 (1975)) and enzyme-linked immunosorbent assays (Engvall and Perlmann, “Enzyme-linked immunosorbent assay (ELISA). Quantitative assay of immunoglobulin” Immunochemistry 8:871-874 (1971)), and two-dimensional (2-D) gel electrophoresis (Patton, Biotechniques 28: 944-957 (2000)). Although these techniques are useful, most have significant drawbacks and limitations. For example, radioactive labels are dangerous and difficult to handle, fluorescent labels have limited capacity for multiplex detection because of limitations on distinguishable labels, and amplification methods can be subject to spurious signal amplification. There is a need for improved detection labels and detection techniques that can detect minute quantities of specific molecules and that can be highly multiplexed.

Analysis of protein expression and presence, such as proteome profiling or proteomics, requires sensitive detection of multiple proteins. Current methods in proteome profiling suggest that there is a shortage of tools necessary for such detection (Haynes and Yates, Proteome profiling-pitfalls and progress. Yeast 17(2):81-87 (2000)). While the techniques of chromatography and capillary electrophoresis are amenable to proteomic studies and have seen significant development efforts (see for example, Krull et al., Specific applications of capillary electrochromatography to biopolymers, including proteins, nucleic acids, peptide mapping, antibodies, and so forth. J Chromatogr A, 887:137-63 (2000), Hage, Affinity chromatography: a review of clinical applications. Clin Chem, 45(5):593-615 (1999), Hage et al., Chromatographic Immunoassays., Anal Chem, 73(07):198 A-205 A, (2001), Krull et al., Labeling reactions applicable to chromatography and electrophoresis of minute amounts of proteins. J Chromatogr B Biomed Sci Appl, 699:173-208 (1997)), the workhorse of the industry remains two dimensional electrophoresis where the two dimensions are isoelectric focusing and molecular size. Haynes and Yates point out the significant shortcomings of the technique but discuss the utility of the method in light of such shortcomings. Hayes and Yates also discuss the techniques of Isotope Coded Affinity Tags (ICAT), LC-LC-MS/MS, and stable isotope labeling techniques (Shevchenko et al., Rapid ‘de novo’ peptide sequencing by a combination of nanoelectrospray, isotopic labeling and a quadrupole/time-of-flight mass spectrometer. Rapid Commun Mass Spectrom 11(9):1015-1024 (1997); Oda et al., Accurate quantitation ofprotein expression and site-specific phosphorylation. Proc Natl Acad Sci USA 96(12):6591-6596 (1999)).

Aebersold et al. (WO 00/11208) have described labels of the composition PRG-L-A, where PRG is a protein reactive group, L is a linker (that may contain isotopically distinguishable composition), and A is an affinity moiety. Aebersold et al. describes a method where the protein reactive group is used to attach the label to a protein, an affinity capture molecule is used to capture the affinity moiety, the remaining proteins are discarded, then the affinity moiety is released and the labeled proteins are detected by mass spectrometry. The method of Aebersold et al. does not involve fragmentation or other modification of the labels or proteins.

The technique of ICAT, where cysteine residues are labeled with heavy or light tags that each contain affinity moieties, in control and tester samples, has received significant interest and holds potential for protein profiling (Gygi et al., Quantitative analysis of complex protein mixtures using isotope-coded affinity tags. Nat. Biotechnol. 17(10):994-999 (1999), Griffin et al., Quantitative proteomic analysis using a MALDI quadrupole time-of-flight mass spectrometer., Anal. Chem., 73:978-986 (2001)). Gygi et al. and Griffin et al. have demonstrated relative profiling of two protein samples, where the two samples are distinguished utilizing linkers containing either eight normal hydrogen or eight heavy hydrogen (deuterium) atoms. The relative concentrations of labeled proteins are determined by ratio of peaks that are separated by the corresponding 8 amu difference in the linker molecules. Current implementations have been limited to two labels. This technique does not involve fragmentation or other modification of the labels or proteins.

Mass spectrometry has been used to detect phosphorylated proteins (DeGnore and Qin, Fragmentation ofphosphopeptides in an ion trap mass spectrometer. J. Am. Soc. Mass Spectrom. 9:1175-1188 (1998); Qin and Chait, Identification and characterization of posttranslational modifications ofproteins by MALDI ion trap mass spectrometry. Anal Chem, 69:4002-9 (1997); Annan et al., A multidimensional electrospray MS-based approach tophosphopeptide mapping. Anal. Chem. 73:393-404 (2001)). The methods make use of a signature mass to indicate the presence of a phosphate group, for example m/z=63 and/or m/z=79 corresponding to PO₂⁻ and PO₃⁻ ions in negative ion mode, or the neutral loss of 98 Daltons from the parent ion indicates the loss of H₃PO₄ from the phosphorylated peptide, indicate phosphorylated Ser, Tyr, Thr. Once phosphorylated amino acids are identified, the peptide containing the modification is sequenced by standard MS/MS techniques. There is a need for a high reliability, highly multiplexed readout system for proteomics.

The status of any living organism may be defined, at any given time in its lifetime, by the complex constellation of proteins that constitute its “proteome.” While the complete status of the proteome could be defined by listing all proteins present (including modified variants) as well as their intracellular locations and concentrations, such a task is beyond the capabilities of any current single analytical method. However, attempts have been made to define the status of a cell or tissue by identifying and measuring the relative concentrations of a small subset of proteins. For example, Conrads et al., Analytical Chemistry, 72:3349-3354 (2000), have described the use of “Accurate Mass Tags” (AMT) for proteome-wide protein identification. Conrads et al. show, for a simple organism, that a mass spectrometer of sufficient mass accuracy and resolution can be used to detect certain tryptic digest fragments from proteins. Once identified, the AMTs may be directly detected in samples by tryptic digest of the proteins, and high accuracy, high resolution mass spectrometry.

While the concept of Accurate Mass Tags is useful for protein discovery, as well as for generating peptide patterns in conventional biological experiments, it does not solve the problem of sensitivity that is at the heart of a truly useful diagnostic multi-protein assessment. A useful assessment consisting of AMTs will require samples containing a minimum of 2000 to 10,000 cells in order to permit reliable readout. This is so because many important cellular proteins are present at levels of only 500 to 5000 molecules per cell. If a clinically relevant protein is present in 500 copies per cell, and a precious clinical sample from a cancer patient contains only 1000 cells, the total number of proteins is 500,000, an amount that lies below the limit of detection by conventional mass spectrometry. Thus, the types of measurements proposed by Conrads et al. for the study of proteomes after identification of AMTs are not suitable for addressing important clinical problems such as the diagnosis of cancer.

### BRIEF SUMMARY OF THE INVENTION

Disclosed are compositions and methods for sensitive detection of one or multiple analytes. In general, the methods involve the use of special label components, referred to as multidimension signals (MDS).

Accordingly, in a first aspect, the invention provides groups of multidimension signals comprising one or more sets of reporter signals, optionally, and one or more indicator signals, wherein the set of reporter signals comprises a plurality of reporter signals, wherein the reporter signals in each set have a common property, wherein the common property allows the reporter signals in the set to be distinguished and/or separated from molecules lacking the common property, wherein the reporter signals can be altered, wherein the altered forms of each reporter signal in each set can be distinguished from every other altered form of reporter signal in the set, wherein the reporter signals and the optional one or more of the indicator signals will generate a predetermined pattern under conditions where the common property allows the reporter signals to be distinguished and/or separated from molecules lacking the common property. Where there is more than one set of reporter signals, the common property in each set of reporter signals is different from the common property in the other sets of reporter signals, wherein the reporter signals will generate a predetermined pattern under conditions where the common property allows the reporter signals to be distinguished and/or separated from molecules lacking the common property.

In a further aspect, the invention provides kits comprising (a) a set of reporter molecules, wherein each reporter molecule comprises a reporter signal and a decoding tag, wherein the reporter signals have a common property, wherein the common property allows the reporter signals to be distinguished and/or separated from molecules lacking the common property, wherein the reporter signals can be altered, wherein the altered forms of each reporter signal can be distinguished from every other altered form of reporter signal, wherein each different reporter molecule comprises a different decoding tag and a different reporter signal, and (b) one or more indicator molecules, wherein each indicator molecule comprises an indicator signal and a decoding tag, wherein the reporter signals and one or more of the indicator signals will generate a predetermined pattern under conditions where the common property allows the reporter signals to be distinguished and/or separated from molecules lacking the common property.

In a yet a further aspect, the invention provides kits comprising two or more sets of reporter molecules, wherein each reporter molecule comprises a reporter signal and a decoding tag, wherein the reporter signals in each set have a common property, wherein the common property allows the reporter signals in the set to be distinguished and/or separated from molecules lacking the common property, wherein the reporter signals can be altered, wherein the altered forms of each reporter signal in each set can be distinguished from every other altered form of reporter signal in the set, wherein each different reporter molecule comprises a different decoding tag and a different reporter signal, wherein the common property in each set of reporter signals is different from the common property in the other sets of reporter signals, wherein the reporter signals will generate a predetermined pattern under conditions where the common property allows the reporter signals to be distinguished and/or separated from molecules lacking the common property.

In various embodiments of all of the aspects of the invention, the reporter signals and indicator signals can comprise peptides, wherein the reporter signals have the same mass-to-charge ratio, wherein at least one of the indicator signals does not have the same mass-to-charge ratio as the reporter signals. In some forms, the indicator signals do not have the common property. The reporter signals and one or more of the indicator signals can generate a predetermined pattern under conditions where the common property allows the reporter signals to be distinguished and/or separated from molecules lacking the common property. The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation.

In various embodiments of all of the aspects of the invention, alteration of the reporter signals can also alter their charge. The common property can be mass-to-charge ratio, wherein the reporter signals can be altered by altering their charge, wherein the altered forms of the labeled proteins can be distinguished via differences in the mass-to-charge ratio of the altered forms of reporter signals.

In various embodiments of all of the aspects of the invention, the set can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The set can comprise ten or more different reporter signals. The reporter signals can be peptides, oligonucleotides, carbohydrates, polymers, oligopeptides, or peptide nucleic acids.

In various embodiments of all of the aspects of the invention, the reporter signals can be associated with, or coupled to, specific binding molecules (e.g., each reporter signal can be associated with, or coupled to, a different specific binding molecule). The reporter signals can be associated with, or coupled to, decoding tags (e.g., each reporter signal can be associated with, or coupled to, a different decoding tag). The reporter signals can be associated with, or coupled to, proteins or peptides. The peptides can have the same amino acid composition, can have the same amino acid sequence, can contain a different distribution of heavy isotopes, can have a different amino acid sequence, or can have a labile or scissile bond in a different location.

In various embodiments of all of the aspects of the invention, tn some forms, the indicator signals do not have the common property.

In a further aspect, the invention provides sets of labeled proteins wherein each labeled protein comprises a protein or peptide and a reporter signal or indicator signal attached to the protein or peptide, wherein the reporter signals have a common property, wherein the common property allows the labeled proteins comprising the same protein or peptide to be distinguished and/or separated from molecules lacking the common property. In some embodiments, the reporter signals can be altered, wherein the altered forms of each reporter signal can be distinguished from every other altered form of reporter signal, wherein alteration of the reporter signals alters the labeled proteins, wherein altered forms of each labeled protein can be distinguished from every other altered form of labeled protein, wherein the reporter signals and one or more of the indicator signals will generate a predetermined pattern under conditions where the common property allows the labeled proteins to be distinguished and/or separated from molecules lacking the common property. In some embodiments, the reporter signals and/or one or more of the indicator signals will generate a predetermined pattern under conditions where the common property allows the labeled proteins to be distinguished and/or separated from molecules lacking the common property.

In another aspect, the invention provides sets of labeled proteins wherein each labeled protein comprises a protein or peptide and a reporter signal or indicator signal attached to the protein or peptide, wherein the reporter signals can be altered, wherein the altered forms of each reporter signal can be distinguished from every other altered form of reporter signal, wherein alteration of the reporter signals alters the labeled proteins, wherein altered forms of each labeled protein can be distinguished from every other altered form of labeled protein, wherein the reporter signals and one or more of the indicator signals will generate a predetermined pattern.

In another aspect, the invention provides sets of labeled proteins wherein each labeled protein comprises a protein or peptide and a reporter signal attached to the protein or peptide, wherein the reporter signals belong to one of two or more sets of reporter signals, wherein the reporter signals in each set have a common property, wherein the common property in each set of reporter signals is different from the common property in the other sets of reporter signals, wherein the common property allows the labeled proteins comprising the same protein or peptide to be distinguished and/or separated from molecules lacking the common property, wherein the reporter signals can be altered, wherein the altered forms of each reporter signal in each set can be distinguished from every other altered form of reporter signal in the set, wherein alteration of the reporter signals alters the labeled proteins, wherein altered forms of each labeled protein can be distinguished from every other altered form of labeled protein, wherein the reporter signals will generate a predetermined pattern under conditions where the common property allows the labeled proteins to be distinguished and/or separated from molecules lacking the common property.

In yet another aspect, the invention provides sets of labeled proteins wherein each labeled protein comprises a protein or peptide and a reporter signal attached to the protein or peptide, wherein the labeled proteins belong to one of two or more sets of labeled proteins, wherein the labeled proteins in each set have a common property, wherein the common property in each set of labeled proteins is different from the common property in the other sets of labeled proteins. In some embodiments, the common property allows the labeled proteins comprising the same protein or peptide to be distinguished and/or separated from molecules lacking the common property, wherein the reporter signals can be altered, wherein the altered forms of each reporter signal in each labeled protein in each set can be distinguished from every other altered form of reporter signal in the labeled proteins in the set, wherein alteration of the reporter signals alters the labeled proteins, wherein altered forms of each labeled protein in each set can be distinguished from every other altered form of labeled protein in the set, wherein the reporter signals will generate a predetermined pattern under conditions where the common property allows the labeled proteins to be distinguished and/or separated from molecules lacking the common property. In some embodiments, the common property allows the labeled protein to be distinguished and/or separated from molecules lacking the common property, wherein the reporter signal can be altered, wherein alteration of the reporter signals alters the labeled protein, wherein altered forms of each labeled protein in each set can be distinguished from every other unaltered form of labeled protein in the set, wherein the reporter signals will generate a predetermined pattern under conditions where the common property allows the labeled proteins to be distinguished and/or separated from molecules lacking the common property.

In a further aspect, the invention provides sets of labeled proteins wherein each labeled protein comprises a protein or peptide and a reporter signal attached to the protein or peptide, wherein the labeled proteins belong to one of two or more sets of labeled proteins, wherein the reporter signals can be altered, wherein the altered forms of each reporter signal in each labeled protein in each set can be distinguished from every other altered form of reporter signal in the labeled proteins in the set, wherein alteration of the reporter signals alters the labeled proteins, wherein altered forms of each labeled protein in each set can be distinguished from every other altered form of labeled protein in the set, wherein the reporter signals will generate a predetermined pattern.

In a yet further aspect, the invention provides kits comprising a set of reporter molecules and one or more indicator molecules, wherein each reporter molecule comprises a reporter signal and a coupling tag, wherein the reporter signals have a common property, wherein the common property allows the reporter signals to be distinguished and/or separated from molecules lacking the common property, wherein the reporter signals can be altered, wherein the altered forms of each reporter signal can be distinguished from every other altered form of reporter signal, wherein each different reporter molecule comprises a different coupling tag and a different reporter signal, wherein each indicator molecule comprises an indicator signal and a coupling tag, wherein the reporter signals and one or more of the indicator signals will generate a predetermined pattern under conditions where the common property allows the reporter signals to be distinguished and/or separated from molecules lacking the common property.

In a further aspect, the invention provides labeled proteins wherein the labeled protein comprises a protein or peptide and a reporter signal or indicator signal attached to the protein or peptide, wherein the labeled protein has a common property, wherein the common property allows the labeled protein to be distinguished and/or separated from molecules lacking the common property, wherein the reporter signal can be altered, wherein alteration of the reporter signals alters the labeled protein, wherein the altered form of the labeled protein can be distinguished from the unaltered form of labeled protein, wherein the reporter signals and one or more of the indicator signals will generate a predetermined pattern under conditions where the common property allows the labeled proteins to be distinguished and/or separated from molecules lacking the common property.

In another aspect, the invention provides kits comprising a set of reporter molecules, wherein each reporter molecule comprises a reporter signal and a coupling tag, wherein the reporter signals belong to one of two or more sets of reporter signals, wherein the reporter signals in each set have a common property, wherein the common property in each set of reporter signals is different from the common property in the other sets of reporter signals, wherein the common property allows the reporter signals to be distinguished and/or separated from molecules lacking the common property, wherein the reporter signals can be altered, wherein the altered forms of each reporter signal in each set can be distinguished from every other altered form of reporter signal in the set, wherein each different reporter molecule comprises a different coupling tag and a different reporter signal, wherein the reporter signals will generate a predetermined pattern under conditions where the common property allows the reporter signals to be distinguished and/or separated from molecules lacking the common property.

In various embodiments of all of the aspects of the invention, the common property can be mass-to-charge ratio, wherein the reporter signals can be altered by altering their mass, wherein the altered forms of the labeled proteins can be distinguished via differences in the mass-to-charge ratio of the altered forms of reporter signals. The mass of the reporter signals can be altered by fragmentation.

In various embodiments of all of the aspects of the invention, the reporter signals can be coupled to the proteins or peptides. The common property can allow the labeled proteins to be distinguished and/or separated from molecules lacking the common property. The common property can be one or more affinity tags associated with the reporter signals. One or more affinity tags can be associated with the reporter signals. Each labeled protein can comprise a protein or a peptide and a reporter signal or indicator signal attached to the protein or peptide, wherein the reporter signals comprise peptides, wherein the reporter signal have the same mass-to-charge ratio, wherein the indicator signals do not have the same mass-to-charge ratio as the reporter signals. The reporter signal peptides can have the same amino acid composition or can have the same amino acid sequence. Each reporter signal peptide can contain a different distribution of heavy isotopes, can contain a different distribution of substituent groups, or can have a different amino acid sequence. Each reporter signal peptide can have a labile or scissile bond in a different location. One or more affinity tags can be associated with the reporter signals.

In a further aspect, the invention provides mixtures comprising a set of reporter signal calibrators, one or more indicator signal calibrators and a set of target protein fragments, wherein each reporter signal calibrator shares a common property with a target protein fragment in the set of target protein fragments, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragments (e.g., each of these) can be distinguished from the other altered forms (e.g., every other altered form) of the target protein fragments, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators and at least one of the indicator signal calibrators can generate a predetermined pattern under conditions that allows the target protein fragments and reporter signal calibrators having the common property to be distinguished and/or separated from molecules lacking the common property.

In a further aspect, the invention provides sets of target protein fragments, wherein each target protein fragment shares a common property with a reporter signal calibrator in a set of reporter signal calibrators, wherein the common property allows the target protein fragments and reporter signal calibrators having the common property to be distinguished and/or separated from molecules lacking the common property, wherein the target protein fragment and reporter signal calibrator that share a common property correspond to each other, wherein the target protein fragments can be altered, wherein the altered forms of the target protein fragments can be distinguished from the other altered forms of the target protein fragments, wherein the reporter signal calibrators can be altered, wherein the altered form of each reporter signal calibrator can be distinguished from the altered form of the target protein fragment with which the reporter signal calibrator shares a common property, wherein the reporter signal calibrators and one or more indicator signal calibrators can generate a predetermined pattern under conditions that allows the target protein fragments and reporter signal calibrators having the common property to be distinguished and/or separated from molecules lacking the common property.

In another aspect, the invention provides sets of reporter signal calibrators and one or more indicator signal calibrators, wherein each reporter signal calibrator shares a common property with a target protein fragment in a set of target protein fragments, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragment (e.g., each of these) can be distinguished from the other altered forms (e.g., every other altered form) of target protein fragment, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators and at least one of the indicator signal calibrators can generate a predetermined pattern under conditions that allows the target protein fragments and reporter signal calibrators having the common property to be distinguished and/or separated from molecules lacking the common property.

In an addition aspect, the invention provides kits for producing a protein signature, the kit comprising (a) a set of reporter signal calibrators and one or more indicator signal calibrators, wherein each reporter signal calibrator shares a common property with a target protein fragment in a set of target protein fragments, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragments (e.g., each of these) can be distinguished from the other altered forms (e.g., every other altered form) of target protein fragment, wherein each of the reporter signal calibrators can be altered, wherein the altered form of each reporter signal calibrator can be distinguished from the altered form of the target protein fragment with which the reporter signal calibrator shares a common property, wherein the reporter signal calibrators and at least one of the indicator signal calibrators can generate a predetermined pattern under conditions that allows the target protein fragments and reporter signal calibrators having the common property to be distinguished and/or separated from molecules lacking the common property, and (b) one or more reagents for treating a protein sample to produce protein fragments.

In another aspect, the invention provides sets of target protein fragments and one or more indicator signal calibrators, wherein each target protein fragment shares a common property with a reporter signal calibrator in a set of reporter signal calibrators, wherein the common property allows each of the target protein fragments and reporter signal calibrators having the common property to be distinguished and/or separated from molecules lacking the common property, wherein the target protein fragment and reporter signal calibrator that share a common property correspond to each other, wherein each of the target protein fragments can be altered, wherein the altered forms of each target protein fragment can be distinguished from every other altered form of target protein fragment, wherein each of the reporter signal calibrators can be altered, wherein the altered form of each reporter signal calibrator can be distinguished from the altered form of the target protein fragment with which the reporter signal calibrator shares a common property, wherein the reporter signal calibrators and at least one of the indicator signal calibrators can generate a predetermined pattern under conditions that allows the target protein fragments and reporter signal calibrators having the common property to be distinguished and/or separated from molecules lacking the common property.

In a further aspect, the invention provides sets of reporter signal calibrators, wherein the reporter signal calibrators belong to one of two or more sets of reporter signal calibrators, wherein each reporter signal calibrator in each set shares a common property with a target protein fragment in a set of target protein fragments, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragment (e.g., each of these) can be distinguished from the other altered forms (e.g., every other altered form) of target protein fragment, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators can generate a predetermined pattern under conditions that allows the target protein fragments and reporter signal calibrators having the common property to be distinguished and/or separated from molecules lacking the common property.

In a further aspect, the invention provides kits for producing a protein signature, the kit comprising (a) two of more sets of reporter signal calibrators, wherein the reporter signal calibrators belong to one of two or more sets of reporter signal calibrators, wherein each reporter signal calibrator in each set shares a common property with a target protein fragment in a set of target protein fragments, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragment (e.g., each of these) can be distinguished from the other altered forms (e.g., every other altered form) of target protein fragment, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators can generate a predetermined pattern under conditions that allows the target protein fragments and reporter signal calibrators having the common property to be distinguished and/or separated from molecules lacking the common property, and (b) one or more reagents for treating a protein sample to produce protein fragments.

In another aspect, the invention provides mixtures comprising two or more sets of reporter signal calibrators and a set of target protein fragments, wherein the reporter signal calibrators belong to one of two or more sets of reporter signal calibrators, wherein each reporter signal calibrator in each set shares a common property with a target protein fragment in the set of target protein fragments, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragments (e.g., each of these) can be distinguished from the other altered forms (e.g., every other altered form) of target protein fragment, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators can generate a predetermined pattern under conditions that allows the target protein fragments and reporter signal calibrators having the common property to be distinguished and/or separated from molecules lacking the common property.

In yet another aspect, the invention provides sets of target protein fragments, wherein each target protein fragment shares a common property with a reporter signal calibrator in a set of reporter signal calibrators, wherein the reporter signal calibrators belong to one of two or more sets of reporter signal calibrators, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the target protein fragments (e.g., each of these) can be altered, wherein the altered forms of the target protein fragments (e.g., each of these) can be distinguished from the other altered forms (e.g., every other altered form) of target protein fragment, wherein the reporter signal calibrators (e.g., each of these) can be altered, wherein the altered form of each reporter signal calibrator can be distinguished from the altered form of the target protein fragment with which the reporter signal calibrator shares a common property, wherein the reporter signal calibrators can generate a predetermined pattern under conditions that allows the target protein fragments and reporter signal calibrators having the common property to be distinguished and/or separated from molecules lacking the common property.

In various embodiments of all of the aspects of the invention, the set can include a predetermined amount of each reporter signal calibrator. The amount of at least two of the reporter signal calibrators can be different. The relative amount each reporter signal calibrator can be based on the relative amount of each corresponding target protein fragment expected to be in the protein sample. The amount of each of the reporter signal calibrators can be the same. The target protein fragments and reporter signal calibrators can be altered by fragmentation. The target protein fragments and reporter signal calibrators can be altered by cleavage at a photocleavable amino acid. The target protein fragments and reporter signal calibrators can be fragmented in a collision cell. The target protein fragments can be fragmented at an aspartic acid-proline bond.

In various embodiments of all of the aspects of the invention, the target protein fragments can be produced by protease digestion of the protein sample. The target protein fragments can be produced by digestion of the protein sample with a serine protease. The serine protease can be trypsin. The target protein fragments can be produced by cleavage at a photocleavable amino acid.

In various embodiments of all of the aspects of the invention, the common property can be mass-to-charge ratio, wherein the target protein fragments and reporter signal calibrators can be altered by altering their mass, their charge, or their mass and charge, wherein the altered forms of the target protein fragments and reporter signal calibrators can be distinguished via differences in the mass-to-charge ratio of the altered forms of the target protein fragments and reporter signal calibrators.

In various embodiments of all of the aspects of the invention, the set of reporter signal calibrators can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signal calibrators. The set of reporter signal calibrators can comprise ten or more different reporter signal calibrators. The set of target protein fragments can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different target protein fragments.

In various embodiments of all of the aspects of the invention, the reporter signal calibrators can comprise peptides, wherein the peptides have the same mass-to-charge ratio as the corresponding target protein fragments. The peptides can have the same amino acid composition as the corresponding target protein fragments. The peptides can have the same amino acid sequence as the corresponding target protein fragments. Each peptide can have a different amino acid sequence than the corresponding target protein fragment. Each peptide can have a labile or scissile bond in a different location.

In various embodiments of all of the aspects of the invention, the reporter signal calibrators can be peptides, oligonucleotides, carbohydrates, polymers, oligopeptides, or peptide nucleic acids. At least one of the target protein fragments can comprise at least one modified amino acid. The modified amino acid can be a phosphorylated amino acid, an acylated amino acid, or a glycosylated amino acid. At least one of the target protein fragments can be the same as the target protein fragment comprising the modified amino acid except for the modified amino acid.

In a further aspect, the invention provides sets of nucleic acid molecules wherein each nucleic acid molecule comprises a nucleotide segment encoding an amino acid segment comprising a reporter signal peptide or indicator signal peptide and a protein or peptide of interest, wherein the reporter signal peptides (or the amino acid segments comprising the reporter signal peptides) have a common property, wherein the common property allows the reporter signal peptides (or the amino acid segments comprising the reporter signal peptides) to be distinguished and/or separated from molecules lacking the common property, wherein the reporter signal peptides can be altered. In some embodiments, the altered form of each reporter signal peptide can be distinguished from the altered forms of the other reporter signal peptides, wherein the reporter signal peptides and one or more of the indicator signal peptides will generate a predetermined pattern under conditions where the common property allows the reporter signal peptides to be distinguished and/or separated from molecules lacking the common property. In some embodiments, alteration of the reporter signal peptides alters the amino acid segments, wherein the altered form of each amino acid segment can be distinguished from the altered forms of the other amino acid segments, wherein the reporter signal peptides and one or more of the indicator signal peptides will generate a predetermined pattern under conditions where the common property allows the reporter signal peptides to be distinguished and/or separated from molecules lacking the common property.

In a further aspect, the invention provides sets of nucleic acid molecules wherein each nucleic acid molecule comprises a nucleotide segment encoding an amino acid segment comprising a reporter signal peptide and a protein or peptide of interest, wherein the reporter signals (or the amino acid segments) belong to one of two or more sets of reporter signals (or belong to one of two or more sets of amino acid segments), wherein the reporter signal peptides (or the amino acid segments) in each set have a common property, wherein the common property in each set of reporter signals (or the amino acid segments) is different from the common property in the other sets of reporter signals (or the amino acid segments), wherein the common property allows the reporter signal peptides (or the amino acid segments) to be distinguished and/or separated from molecules lacking the common property, wherein the reporter signal peptides can be altered, wherein, e.g., the altered form of each reporter signal peptide can be distinguished from the altered forms of the other reporter signal peptides or wherein alteration of the reporter signal peptides alters the amino acid segments, wherein the altered form of each amino acid segment can be distinguished from the altered forms of the other amino acid segments, and wherein the reporter signal peptides (or the amino acid segments) will generate a predetermined pattern under conditions where the common property allows the reporter signal peptides (or the amino acid segments) to be distinguished and/or separated from molecules lacking the common property.

In another aspect, the invention provides sets of nucleic acid molecules wherein each nucleic acid molecule comprises a nucleotide segment encoding an amino acid segment comprising a reporter signal peptide or indicator signal peptide and a protein or peptide of interest, wherein the amino acid segments each comprise an amino acid subsegment, wherein each amino acid subsegment comprises a portion of the protein or peptide of interest and all or a portion of the reporter signal peptide or indicator signal peptide, wherein the amino acid subsegments (e.g., those comprising all or a portion of the reporter signal peptide) have a common property, wherein the common property allows the amino acid subsegments (e.g., those comprising all or a portion of the reporter signal peptide) to be distinguished and/or separated from molecules lacking the common property, wherein the reporter signal peptides can be altered, wherein e.g., the altered form of each reporter signal peptide can be distinguished from the altered forms of the other reporter signal peptides or wherein alteration of the reporter signal peptides alters the amino acid subsegments, wherein the altered form of each amino acid subsegment can be distinguished from the altered forms of the other amino acid subsegments, andwherein the amino acid subsegrnents will generate a predetermined pattern under conditions where the common property allows the amino acid segments comprising all or a portion of the reporter signal peptide to be distinguished and/or separated from molecules lacking the common property.

In another aspect, the invention provides sets of nucleic acid molecules wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments belong to one of two or more sets of amino acid subsegments, wherein the amino acid subsegments in each set have a common property, wherein the common property in each set of amino acid subsegments is different from the common property in the other sets of amino acid subsegments. In some embodiments, the common property allows the amino acid subsegments comprising all or a portion of the reporter signal peptide to be distinguished and/or separated from molecules lacking the common property, wherein the reporter signal peptides can be altered, wherein the altered form of each reporter signal peptide can be distinguished from the altered forms of the other reporter signal peptides, wherein the amino acid subsegments will generate a predetermined pattern under conditions where the common property allows the amino acid segments comprising all or a portion of the reporter signal peptide to be distinguished and/or separated from molecules lacking the common property. In some embodiments, the common property allows the amino acid subsegments to be distinguished and/or separated from molecules lacking the common property, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the amino acid subsegments will generate a predetermined pattern under conditions where the common property allows the amino acid segments comprising all or a portion of the reporter signal peptide to be distinguished and/or separated from molecules lacking the common property.

In a further aspect, the invention provides sets of amino acid segments wherein each amino acid segment comprises a reporter signal peptide and a protein or peptide of interest, wherein the reporter signal peptides belong to one of two or more sets of reporter signal peptides, wherein the reporter signal peptides in each set have a common property, wherein the common property in each set of reporter signal peptides is different from the common property in the other sets of reporter signal peptides, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides will generate a predetermined pattern under conditions where the common property allows the reporter signal peptides to be distinguished and/or separated from molecules lacking the common property.

In a further aspect, the invention provides sets of amino acid segments wherein each amino acid segment comprises a reporter signal peptide or indicator signal peptide and a protein or peptide of interest, wherein the reporter signal peptides have a common property, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides and one or more of the indicator signal peptides will generate a predetermined pattern under conditions where the common property allows the reporter signal peptides to be distinguished and/or separated from molecules lacking the common property.

In a further aspect, the invention provides cells and sets of cells wherein each cell or each cell in the set comprises a nucleic acid molecule wherein each nucleic acid molecule comprises a nucleotide segment encoding an amino acid segment comprising a reporter signal peptide or indicator signal peptide and a protein or peptide of interest, wherein the reporter signal peptides have a common property, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides and one or more of the indicator signal peptides will generate a predetermined pattern under conditions where the common property allows the reporter signal peptides to be distinguished and/or separated from molecules lacking the common property.

In another aspect, the invention provides cells comprising a set of nucleic acid molecules wherein each nucleic acid molecule comprises a nucleotide segment encoding an amino acid segment comprising a reporter signal peptide and a protein or peptide of interest, wherein the reporter signal peptides belong to one of two or more sets of reporter signal peptides, wherein the reporter signal peptides in each set have a common property, wherein the common property in each set of reporter signal peptides is different from the common property in the other sets of reporter signal peptides, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides will generate a predetermined pattern under conditions where the common property allows the reporter signal peptides to be distinguished and/or separated from molecules lacking the common property.

In a further aspect, the invention provides sets of cells or organisms wherein each cell or each organism comprises a nucleic acid molecule wherein each nucleic acid molecule comprises a nucleotide segment encoding an amino acid segment comprising a reporter signal peptide and a protein or peptide of interest, wherein the reporter signal peptides belong to one of two or more sets of reporter signal peptides, wherein the reporter signal peptides in each set have a common property, wherein the common property in each set of reporter signal peptides is different from the common property in the other sets of reporter signal peptides, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides will generate a predetermined pattern under conditions where the common property allows the reporter signal peptides to be distinguished and/or separated from molecules lacking the common property.

In another aspect, the invention provides organisms or sets of organisms wherein the organisms or each organism of the set comprises a nucleic acid molecule wherein each nucleic acid molecule comprises a nucleotide segment encoding an amino acid segment comprising a reporter signal peptide or indicator signal peptide and a protein or peptide of interest, wherein the reporter signal peptides have a common property, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the reporter signal peptides and one or more of the indicator signal peptides will generate a predetermined pattern under conditions where the common property allows the reporter signal peptides to be distinguished and/or separated from molecules lacking the common property.

In various embodiments of all of the aspects of the invention, each nucleic acid molecule can further comprise expression sequences, wherein the expression sequences can be operably linked to the nucleotide segment such that the amino acid segment can be expressed. The expression sequences of each nucleic acid molecule can be different. The different expression sequences can be differently regulated. The expression sequences can be similarly regulated. A plurality of the expression sequences can be expression sequences of, or derived from, genes expressed as part of the same expression cascade. The expression sequences can comprise translation expression sequences and/or transcription expression sequences. The amino acid segment can be expressed in vitro or in vivo. The amino acid segment can be expressed in cell culture. The expression sequences of each nucleic acid molecule can be the same. The expression sequences of at least two nucleic acid molecules can be different or the same. Each nucleic acid molecule can further comprise replication sequences, wherein the replication sequences allow replication of the nucleic acid molecules.

In various embodiments of all of the aspects of the invention, the nucleic acid molecules can be replicated in vitro or in vivo. The nucleic acid molecules can be replicated in cell culture. Each nucleic acid molecule can further comprise integration sequences, wherein the integration sequences allow integration of the nucleic acid molecules into other nucleic acids. The nucleic acid molecules can be integrated into a chromosome (e.g., at a predetermined location). The nucleic acids molecules can be produced by replicating nucleic acids in one or more nucleic acid samples. The nucleic acids can be replicated using pairs of primers, wherein each of the first primers in the primer pairs used to produce the nucleic acid molecules can comprise a nucleotide sequence encoding the reporter signal peptide. Each first primer can further comprise expression sequences. The nucleotide sequence of each first primer can also encode an epitope tag.

In various embodiments of all of the aspects of the invention, each amino acid segment can further comprise an epitope tag. The epitope tag of each amino acid segment can be different or the same. The epitope tag of at least two amino acid segments can be different or the same. The reporter signal peptide of each amino acid segment can be different or the same. The reporter signal peptide of at least two amino acid segments can be different or the same.

In various embodiments of all of the aspects of the invention, the nucleic acid molecules can be in cells or in cell lines. Each nucleic acid molecule can be in a different cell (or cell line) or in the same cell (or cell line). The nucleic acid molecules can be in organisms. Each nucleic acid molecule can be in a different organism, or in the same organism. The nucleic acid molecules can be integrated into a chromosome (e.g., at a predetermined location) of the cell or organism. The chromosome can be an artificial chromosome. The nucleic acid molecules can be, or can be integrated into, a plasmid. The nucleic acid molecules can be in cells of an organism (e.g., in substantially all of the cells of the organism or in some of the cells of the organism). The amino acid segments can be expressed in substantially all of the cells of the organism or can be expressed in some of the cells of the organism.

In various embodiments of all of the aspects of the invention, the protein or peptide of interest of each amino acid segment can be different or the same. The protein or peptide of interest of at least two amino acid segments can be different or the same. The proteins or peptides of interest can be related, can be proteins produced in the same cascade, can be proteins in the same enzymatic pathway, can be proteins expressed under the same conditions, or can be proteins associated with the same disease, can be proteins associated with the same cell type or the same tissue type.

In various embodiments of all of the aspects of the invention, the nucleotide segment can encode a plurality of amino acid segments each comprising a reporter signal peptide or indicator signal peptide and a protein or peptide of interest. The protein or peptide of interest of at least two of the amino acid segments in one of the nucleotide segments can be different. The protein or peptide of interest of the amino acid segments in one of the nucleotide segments can be different. The protein or peptide of interest of at least two of the amino acid segments in each of the nucleotide segments can be different. The protein or peptide of interest of the amino acid segments in each of the nucleotide segments can be different.

In various embodiments of all of the aspects of the invention, the set of nucleic acid molecules can consist of a single nucleic acid molecule. The nucleic acid molecule can comprise a plurality of nucleotide segments each encoding an amino acid segment. The amino acid segment can comprise a cleavage site near the junction between the reporter signal peptide and the protein or peptide of interest. The cleavage site can be a trypsin cleavage site. The cleavage site can be at the junction between the reporter signal peptide and the protein or peptide of interest. Each amino acid segment can further comprise a self-cleaving segment. The self-cleaving segment can be between the reporter signal peptide and the protein or peptide of interest. The self-cleaving segment can be an intein segment.

In various embodiments of all of the aspects of the invention, the amino acid segment can be a protein or peptide. The set of amino acid segments can consist of a single amino acid segment, wherein the amino acid segment comprises a plurality of reporter signal peptides.

In various embodiments of all of the aspects of the invention, each cell or organism can further comprise additional nucleic acid molecules. The set of cells can consist of a single cell, wherein the cell comprises a plurality of nucleic acid molecules. The set can consist of a single cell, wherein the cell comprises a set of nucleic acid molecules, wherein the set of nucleic acid molecules consists of a single nucleic acid molecule, wherein the nucleic acid molecule encodes a plurality of nucleic acid segments. Similarly, the set of organisms can consist of a single organism, wherein the organism comprises a plurality of nucleic acid molecules. The set can consist of a single organism, wherein the organism comprises a set of nucleic acid molecules, wherein the set of nucleic acid molecules consists of a single nucleic acid molecule, wherein the nucleic acid molecule encodes a plurality of nucleic acid segments.

In a further aspect, the invention provides methods comprising (a) separating one or more reporter signals and one or more indicator signals, where each reporter signal has a common property, from molecules lacking the common property in one sample or in each of a plurality of samples, (b) identifying a predetermined pattern generated by the reporter signals and one or more of the indicator signals, (c) altering the reporter signals that generate the predetermined pattern, (d) detecting and distinguishing the altered forms the reporter signals from each other.

In a further aspect, the invention provides methods comprising (a) separating two or more sets of reporter signals, where the reporter signals in each set have a common property, wherein the common property in each set of reporter signals is different from the common property in the other sets of reporter signals, from molecules lacking the common property in one sample or in each of a plurality of samples, (b) identifying a predetermined pattern generated by the reporter signals, (c) altering the reporter signals that generate the predetermined pattern, (d) detecting and distinguishing the altered forms the reporter signals from each other.

In some forms of the invention, the indicator signals do not have the common property. The set of reporter signals and one or more of the indicator signals can generate a predetermined pattern under conditions where the common property allows the reporter signals to be distinguished and/or separated from molecules lacking the common property. The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. The set of reporter signals can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The set of reporter signals can comprise ten or more different reporter signals.

In various embodiments of all of the aspects of the invention, the reporter signals can be peptides, oligonucleotides, carbohydrates, polymers, oligopeptides, or peptide nucleic acids. The reporter signals can be associated with, or coupled to, specific binding molecules, wherein each reporter signal can be associated with, or coupled to, a different specific binding molecule. The reporter signals can be associated with, or coupled to, decoding tags, wherein each reporter signal can be associated with, or coupled to, a different decoding tag.

In various embodiments of all of the aspects of the invention, the methods can further comprise, prior to step (a), associating the reporter signals with one or more analytes, wherein each reporter signal can be associated with, or coupled to, a different specific binding molecule, wherein each specific binding molecule can interact specifically with a different one of the analytes, wherein the reporter signals can be associated with the analytes via interaction of the specific binding molecules with the analytes. Steps (a) through (d) can be repeated one or more times using a different set of one or more reporter signals each time (where the same or a different set of indicator signals can be used each time). Prior to step (a), the different sets of reporter signals can be associated with different samples.

In various embodiments of all of the aspects of the invention, the different sets of reporter signals each can comprise the same reporter signals. The sets of reporter signals each can contain a single reporter signal. Not all of the reporter signals in the set need be or are distinguished and/or separated from molecules lacking the common property, not all of the reporter signals need be or are altered, and not all of the altered forms of the reporter signals need be or are detected at the same time. All of the reporter signals in the set can be distinguished and/or separated from molecules lacking the common property, all of the reporter signals can be altered, and all of the altered forms of the reporter signals can be detected at different times.

In various embodiments of all of the aspects of the invention, steps (a) through (d) can be performed separately for each reporter signal. The reporter signals can comprise peptides, wherein the peptides have the same mass-to-charge ratio. The peptides can have the same amino acid composition or the same amino acid sequence. Each peptide can contain a different distribution of heavy isotopes, can have a different amino acid sequence, or can have a labile or scissile bond in a different location.

In various embodiments of all of the aspects of the invention, the set of reporter signals and one or more of the indicator signals can generate a predetermined pattern under conditions where the common property allows the reporter signals to be distinguished and/or separated from molecules lacking the common property. Not all of the reporter signals need be or are distinguished and/or separated from molecules lacking the common property, not all of the reporter signals need be or are altered, and not all of the altered forms of the reporter signals need be or are detected at the same time. All of the reporter signals can be distinguished and/or separated from molecules lacking the common property, all of the reporter signals can be altered, and all of the altered forms of the reporter signals can be detected at different times.

In a further aspect, the invention provides methods comprising either (a) separating one or more labeled proteins, wherein each labeled protein comprises a protein or peptide and a reporter signal attached to the protein or peptide, wherein the reporter signals belong to one of two or more sets of reporter signals, wherein each reporter signal has a common property, wherein the common property in each set of reporter signals is different from the common property in the other sets of reporter signals, wherein the common property allows the labeled proteins comprising the same protein or peptide to be distinguished and/or separated from molecules lacking the common property (e.g., in each of one or more samples), (a) separating one or more labeled proteins, wherein each labeled protein comprises a protein or peptide and a reporter signal or indicator signal attached to the protein or peptide, wherein each reporter signal has a common property, wherein the common property allows the labeled proteins comprising the same protein or peptide to be distinguished and/or separated from molecules lacking the common property (e.g., in each of one or more samples), or (a) separating one or more labeled proteins from other molecules, wherein the labeled proteins can be derived from one or more samples, wherein each labeled protein comprises a protein or peptide and a reporter signal or a reporter signal or indicator signal attached to the protein or peptide, (b) identifying a predetermined pattern generated by the reporter signals and, if present, one or more of the indicator signals, (c) altering the reporter signals that generate the predetermined pattern, thereby altering the labeled proteins, and (d) detecting and distinguishing the altered forms the labeled proteins from each other.

In a further aspect, the invention provides methods comprising (a) separating a set of labeled proteins, wherein each labeled protein comprises a protein or peptide and a reporter signal or indicator signal attached to the protein or peptide, wherein each labeled protein has a common property, wherein the common property allows the labeled proteins comprising the same protein or peptide to be distinguished and/or separated from molecules lacking the common property, (b) identifying a predetermined pattern generated by the reporter signals and one or more of the indicator signals, (c) altering the reporter signals that generate the predetermined pattern, thereby altering the labeled proteins, (d) detecting and distinguishing the altered forms of the labeled proteins from each other.

In a further aspect, the invention provides methods comprising (a) altering one or more labeled proteins, wherein the labeled protein comprises a protein or peptide and a reporter signal or indicator signal attached to the protein or peptide, wherein the labeled proteins can be altered by altering the reporter signals, (b) detecting and distinguishing the altered forms of the labeled protein from the unaltered form of labeled protein or, where more than one labeled protein was altered, from each other, wherein the reporter signals and/or one or more of the indicator signals will generate a predetermined pattern. In some embodiments, the method is used to detect a protein or peptide.

In a further aspect, the invention provides methods comprising (a) separating a set of labeled proteins, wherein each labeled protein comprises a protein or peptide and a reporter signal attached to the protein or peptide, wherein the labeled proteins belong to one of two or more sets of labeled proteins, wherein each labeled protein has a common property, wherein the common property in each set of labeled proteins is different from the common property in the other sets of labeled proteins, wherein the common property allows the labeled proteins comprising the same protein or peptide to be distinguished and/or separated from molecules lacking the common property, (b) identifying a predetermined pattern generated by the reporter signals, (c) altering the reporter signals that generate the predetermined pattern, thereby altering the labeled proteins, (d) detecting and distinguishing the altered forms of the labeled proteins from each other.

In a further aspect, the invention provides methods of detecting a protein, the methods comprising, detecting a labeled protein, wherein the labeled protein comprises a protein or peptide and a reporter signal or either a reporter signal or indicator signal attached to the protein or peptide, wherein the labeled protein is altered by altering the reporter signal, detecting an altered form of the labeled protein, wherein the labeled protein is altered by altering the reporter signal, and identifying the protein based on the characteristics of the labeled protein and altered form of the labeled protein, wherein the reporter signals and, if present, one or more of the indicator signals will generate a predetermined pattern.

In a further aspect, the invention provides catalogs of proteins and peptides comprising, proteins and peptides in one or more samples detected by (a) separating one or more labeled proteins from other molecules, wherein the labeled proteins can be derived from the one or more samples, wherein each labeled protein comprises a protein or peptide and a reporter signal or indicator signal attached to the protein or peptide, (b) identifying a predetermined pattern generated by the reporter signals and/or one or more of the indicator signals, (c) altering the reporter signals that generate the predetermined pattern, thereby altering the labeled proteins, (d) detecting and distinguishing the altered forms the labeled proteins from each other.

In various embodiments of all of the aspects of the invention, the common property can be mass-to-charge ratio, wherein the reporter signals can be altered by altering their mass, wherein the altered forms of the labeled proteins can be distinguished via differences in the mass-to-charge ratio of the altered forms of the labeled proteins. The mass of the reporter signals can be altered by fragmentation. Alteration of the reporter signals also can alter their charge. The common property can be mass-to-charge ratio, wherein the reporter signals can be altered by altering their charge, wherein the altered forms of the labeled proteins can be distinguished via differences in the mass-to-charge ratio of the altered forms of reporter signals. The set of labeled proteins can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signals. The set of labeled proteins can comprise ten or more different reporter signals.

In various embodiments of all of the aspects of the invention, the reporter signals can be peptides, oligonucleotides, carbohydrates, polymers, oligopeptides, or peptide nucleic acids. The reporter signals can be coupled to the proteins or peptides. Steps (a) through (d) can be performed separately for each labeled protein. The method can further comprise, prior to step (a), attaching the reporter signals to one or more proteins, one or more peptides, or one or more proteins and peptides. Steps can be repeated one or more times using a different set of one or more reporter signals each time (wherein the same or a different set of indicator signals can be used each time). Prior to step (a), the different sets of reporter signals can be attached to proteins or peptides in different samples. The different sets of reporter signals each can comprise the same reporter signals. The sets of reporter signals each can contain a single reporter signal.

In various embodiments of all of the aspects of the invention, it will be understood that not all of the labeled proteins in the set need be or are distinguished and/or separated from molecules lacking the common property, not all of the reporter signals need be or are altered, and not all of the altered forms of the labeled proteins need be or are detected at the same time. All of the labeled proteins in the set can be distinguished and/or separated from molecules lacking the common property, all of the reporter signals can be altered, and all of the altered forms of the labeled proteins can be detected at different times. Steps (a) through (d) can be performed separately for each reporter signal.

In various embodiments of all of the aspects of the invention, the common property can be one or more affinity tags associated with the reporter signals. One or more affinity tags can be associated with the reporter signals. The collection of altered forms of the labeled proteins detected can constitute a catalog of proteins. Steps (a) through (d) can be performed separately for each sample. The different samples can be from the same protein sample. The different samples can be obtained at different times, can be from the same type of organism, can be from the same type of tissue, can be from the same organism, or can be obtained at different times.

In various embodiments of all of the aspects of the invention, the different samples can be from different organisms, from different types of tissues, from different species of organisms, from different strains of organisms, or from different cellular compartments. The method can further comprise identifying or preparing proteins or peptides corresponding to the proteins or peptides present in one sample but not present in another sample. The method can further comprise determining the relative amount of proteins or peptides in the different samples.

In various embodiments of all of the aspects of the invention, the pattern of the presence, amount, presence and amount, or absence of labeled proteins in one of the samples can constitute a catalog of proteins in the sample. The pattern of the presence, amount, presence and amount, or absence of labeled proteins in a second one of the samples can constitute a catalog of proteins in the second sample, wherein the catalog of proteins in the first sample is a first catalog and the catalog of proteins in the second sample is a second catalog, the method can further comprise comparing the first catalog and the second catalog.

In various embodiments of all of the aspects of the invention, each labeled protein can comprise a protein or a peptide and a reporter signal or indicator signal attached to the protein or peptide, wherein the reporter signals comprise peptides, wherein the reporter signals have the same mass-to-charge ratio, wherein the indicator signals do not have the same mass-to-charge ratio as the reporter signals. The reporter signal peptides can have the same amino acid composition or the same amino acid sequence. Each reporter signal peptide can contain a different distribution of heavy isotopes, can contain a different distribution of substituent groups, can have a different amino acid sequence, or can have a labile or scissile bond in a different location.

In various embodiments of all of the aspects of the invention, the method can further comprise, detecting the unaltered form of labeled protein. The labeled protein and altered form of the labeled protein can be detected by detecting the mass-to-charge ratio of the labeled protein and the mass-to-charge ratio of the altered form of the labeled protein or the mass-to-charge ratio of the altered form of the reporter signal. The method can firther comprise, prior to step (a), associating one or more reporter signals and one or more indicator signals with one or more proteins, one or more peptides, or one or more proteins and peptides from each of the one or more samples, wherein the reporter signals and one or more of the indicator signals will generate a predetermined pattern.

In various embodiments of all of the aspects of the invention, the different sets of reporter signals each can comprise the same reporter signals. Each reporter signal or each labeled protein can have a common property, wherein the common property allows the labeled proteins comprising the same protein or peptide to be distinguished and/or separated from molecules lacking the common property. The one or more labeled proteins can be derived from a single sample. A single labeled protein can be distinguished and/or separated from other molecules. A plurality of labeled proteins can be distinguished and/or separated from other molecules.

In various embodiments of all of the aspects of the invention, the detected altered forms of the labeled proteins constitute a catalog of proteins in the sample. One or more labeled proteins can be derived from each of a plurality of samples. A single labeled protein derived from each of the samples can be distinguished and/or separated from other molecules. A plurality of labeled proteins derived from each of the samples can be distinguished and/or separated from other molecules. The detected altered forms of the labeled proteins derived from each sample can constitute a catalog of proteins in the sample.

In a further aspect, the invention provides methods of producing a protein signature, the method comprising (a) treating a protein sample to produce protein fragments, wherein the protein fragments comprise a set of target protein fragments, wherein the target protein fragments (e.g., each of these) can be altered, wherein the altered forms of the target protein fragments can be distinguished from the other altered forms of the target protein fragments, (b) mixing the target protein fragments with a set of reporter signal calibrators and one or more indicator signal calibrators, wherein each target protein fragment shares a common property with at least one of the reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the reporter signal calibrators (e.g., each of these) can be altered, wherein the altered form of each reporter signal calibrator can be distinguished from the altered form of the target protein fragment with which the reporter signal calibrator shares a common property, (c) separating the target protein fragments and reporter signal calibrators from other molecules based on the common properties of the target protein fragments and reporter signal calibrators, (d) identifying a predetermined pattern generated by the reporter signal calibrators and one or more of the indicator signal calibrators, (e) altering the target protein fragments and reporter signal calibrators that generated the predetermined pattern, and (f) detecting the altered forms of the target protein fragments and reporter signal calibrators, wherein the presence, absence, amount, or presence and amount of the altered forms of the target protein fragments indicates the presence, absence, amount, or presence and amount in the protein sample of the target protein fragments from which the altered forms of the target protein fragments are derived, wherein the presence, absence, amount, or presence and amount of the target protein fragments in the protein sample constitutes a protein signature of the protein sample.

In a further aspect, the invention provides methods of producing a protein signature, the method comprising (a) treating a protein sample to produce protein fragments, wherein the protein fragments comprise a set of target protein fragments, wherein the target protein fragments can be altered, wherein the altered forms of the target protein fragments (e.g., each of these) can be distinguished from the other altered forms of the target protein fragments, (b) mixing the target protein fragments with two or more sets of reporter signal calibrators, wherein the reporter signal calibrators belong to one of the two or more sets of reporter signal calibrators, wherein each target protein fragment shares a common property with at least one of the reporter signal calibrators, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the reporter signal calibrators (e.g., each of these) can be altered, wherein the altered form of each reporter signal calibrator can be distinguished from the altered form of the target protein fragment with which the reporter signal calibrator shares a common property, (c) separating the target protein fragments and reporter signal calibrators from other molecules based on the common properties of the target protein fragments and reporter signal calibrators, (d) identifying a predetermined pattern generated by the reporter signal calibrators, (e) altering the target protein fragments and reporter signal calibrators that generated the predetermined pattern, (f) detecting the altered forms of the target protein fragments and reporter signal calibrators, wherein the presence, absence, amount, or presence and amount of the altered forms of the target protein fragments indicates the presence, absence, amount, or presence and amount in the protein sample of the target protein fragments from which the altered forms of the target protein fragments are derived, wherein the presence, absence, amount, or presence and amount of the target protein fragments in the protein sample constitutes a protein signature of the protein sample.

In another aspect, the invention provides methods of producing a protein signature, the method comprising identifying a predetermined pattern generated by reporter signal calibrators and one or more indicator signal calibrators, and detecting altered forms of target protein fragments and the reporter signal calibrators, wherein the altered forms of the target protein fragments (e.g., each of these) can be distinguished from the other altered forms (e.g., every other altered form) of the target protein fragments, wherein each target protein fragment shares a common property with at least one of the reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the altered form of each reporter signal calibrator can be distinguished from the altered form of the target protein fragment with which the reporter signal calibrator shares a common property, wherein the presence, absence, amount, or presence and amount of the altered forms of the target protein fragments indicates the presence, absence, amount, or presence and amount in a protein sample of the target protein fragments from which the altered forms of the target protein fragments are derived, wherein the presence, absence, amount, or presence and amount of the target protein fragments in the protein sample constitutes a protein signature of the protein sample. In some embodiments, the reporter signal calibrators and one or more of the indicator signal calibrators will generate the predetermined pattern under conditions where the common property allows the reporter signal calibrators to be distinguished and/or separated from molecules lacking the common property.

In a further aspect, the invention provides methods of producing a protein signature, the method comprising identifying a predetermined pattern generated by reporter signal calibrators, and detecting altered forms of target protein fragments and the reporter signal calibrators, wherein the altered forms of the target protein fragments (e.g., each of these) can be distinguished from the other altered forms (e.g., every other altered form) of the target protein fragments, wherein the reporter signal calibrators belong to one of two or more sets of reporter signal calibrators, wherein each target protein fragment shares a common property with at least one of the reporter signal calibrators, wherein the common property in each set of reporter signal calibrators is different from the common property in the other sets of reporter signal calibrators, wherein the common property allows the target protein fragments (e.g., each of these) and reporter signal calibrators having the common property to be distinguished and/or separated from molecules lacking the common property, wherein the target protein fragment and reporter signal calibrator that share a common property correspond to each other, wherein the altered form of each reporter signal calibrator can be distinguished from the altered form of the target protein fragment with which the reporter signal calibrator shares a common property, wherein the presence, absence, amount, or presence and amount of the altered forms of the target protein fragments indicates the presence, absence, amount, or presence and amount in a protein sample of the target protein fragments from which the altered forms of the target protein fragments are derived, wherein the presence, absence, amount, or presence and amount of the target protein fragments in the protein sample constitutes a protein signature of the protein sample. In some embodiments, the reporter signal calibrators will generate the predetermined pattern under conditions where the common property allows the reporter signal calibrators to be distinguished and/or separated from molecules lacking the common property.

In another aspect, the invention provides methods of producing a protein signature, the method comprising (a) treating a protein sample to produce protein fragments, wherein the protein fragments comprise a set of target protein fragments, wherein the target protein fragments (e.g., each of these) can be altered, wherein the altered forms of the target protein fragments (e.g., each of these) can be distinguished from the other altered forms (e.g., every other altered form) of the target protein fragments, (b) separating the target protein fragments from other protein fragments in the protein sample, (c) identifying a predetermined pattern generated by the target protein fragments and, optionally, one or more indicator signal calibrators, (d) altering the target protein fragments that generated the predetermined pattern, and (e) detecting the altered forms of the target protein fragments, wherein the presence, absence, amount, or presence and amount of the altered forms of the target protein fragments indicates the presence, absence, amount, or presence and amount in the protein sample of the target protein fragments from which the altered forms of the target protein fragments are derived, wherein the presence, absence, amount, or presence and amount of the target protein fragments in the protein sample constitutes a protein signature of the protein sample.

In another aspect, the invention provides methods of producing a protein signature, the method comprising (a) separating a plurality of target protein fragments from other protein fragments in a protein sample, (b) identifying a predetermined pattern generated by the target protein fragments and, optionally, one or more indicator signal calibrators, (c) altering the target protein fragments that generated the predetermined pattern, (d) detecting the altered forms of the target protein fragments, wherein the presence, absence, amount, or presence and amount of the altered forms of the target protein fragments indicates the presence, absence, amount, or presence and amount in the protein sample of the target protein fragments from which the altered forms of the target protein fragments are derived, wherein the presence, absence, amount, or presence and amount of the target protein fragments in the protein sample constitutes a protein signature of the protein sample.

In a further aspect, the invention provides methods of analyzing a protein sample, the method comprising (a) mixing a protein sample with a predetermined amount of a reporter signal calibrator and one or more indicator signal calibrators, wherein the protein sample has a known amount of protein, wherein the protein sample comprises a target protein fragment, wherein the target protein fragment can be altered, wherein the reporter signal calibrator can be altered, wherein the altered form of the reporter signal calibrator can be distinguished from the altered form of the target protein fragment, (b) identifying a predetermined pattern generated by the reporter signal calibrator and one or more of the indicator signal calibrators, (c) altering the target protein fragment and reporter signal calibrator that generated the predetermined pattern, (d) detecting the altered forms of the target protein fragment and reporter signal calibrator.

In a further aspect, the invention provides methods of analyzing a protein sample, the method comprising (a) mixing a protein sample with a predetermined amount of two or more reporter signal calibrators, wherein the protein sample has a known amount of protein, wherein the protein sample comprises a target protein fragment, wherein the target protein fragment can be altered, wherein the reporter signal calibrator can be altered, wherein the altered form of the reporter signal calibrator can be distinguished from the altered form of the target protein fragment, (b) identifying a predetermined pattern generated by the reporter signal calibrators, (c) altering the target protein fragment and reporter signal calibrator that generated the predetermined pattern, (d) detecting the altered forms of the target protein fragment and reporter signal calibrator.

In an additional aspect, the invention provides methods of analyzing a protein sample, the method comprising (a) treating a protein sample to produce protein fragments, wherein the protein sample has a known amount of protein, wherein the protein sample comprises a target protein, wherein the protein fragments comprise a target protein fragment derived from the target protein, (b) mixing the protein sample with a predetermined amount of a reporter signal calibrator and one or more indicator signal calibrators, wherein the target protein fragment can be altered, wherein the reporter signal calibrator can be altered, wherein the altered form of the reporter signal calibrator can be distinguished from the altered form of the target protein fragment, (c) identifying a predetermined pattern generated by the reporter signal calibrator and one or more of the indicator signal calibrators, (d) altering the target protein fragment and reporter signal calibrator that generated the predetermined pattern, (e) detecting the altered forms of the target protein fragment and reporter signal calibrator.

In a further aspect, the invention provides methods of analyzing a protein sample, the method comprising (a) treating a protein sample to produce protein fragments, wherein the protein sample has a known amount of protein, wherein the protein sample comprises a target protein, wherein the protein fragments comprise a target protein fragment derived from the target protein, (b) mixing the protein sample with a predetermined amount of two or more reporter signal calibrators, wherein the reporter signal calibrators belong to one of two or more sets of reporter signal calibrators, wherein the target protein fragment can be altered, wherein the reporter signal calibrator can be altered, wherein the altered form of the reporter signal calibrator can be distinguished from the altered form of the target protein fragment, (c) identifying a predetermined pattern generated by the reporter signal calibrators, (d) altering the target protein fragment and reporter signal calibrator that generated the predetermined pattern, (e) detecting the altered forms of the target protein fragment and reporter signal calibrator.

In some forms, the indicator signal calibrators do not have the common property. The reporter signal calibrators and one or more of the indicator signal calibrators can generate a predetermined pattern under conditions where the common property allows the reporter signal calibrators to be distinguished and/or separated from molecules lacking the common property. Steps (e) and (f) can be performed simultaneously. The altered forms of the target protein fragments can be detecting using mass spectrometry. Steps (c), (d), (e) and (f) can be performed with a tandem mass spectrometer.

In various embodiments of all of the aspects of the invention, the tandem mass spectrometer can comprise a first stage and a last stage, wherein step (c) can be performed using the first stage of the tandem mass spectrometer to select ions in a narrow mass-to-charge range, wherein step (e) can be performed by collision with a gas, and wherein step (f) can be performed using the final stage of the tandem mass spectrometer. The first stage of the tandem mass spectrometer can be a quadrupole mass filter. The final stage of the tandem mass spectrometer can be a time of flight analyzer. The final stage of the tandem mass spectrometer can be a time of flight analyzer. The mass-to-charge range can be varied to cover the mass-to-charge ratio of each of the target protein fragments.

In various embodiments of all of the aspects of the invention, it will be understood that a predetermined amount of each reporter signal calibrator can be mixed with the target protein fragments, wherein the amount of each altered form of reporter signal calibrator detected can provide a standard for assessing the amount of the altered form of the corresponding target protein fragment. The amount of at least two of the reporter signal calibrators can be different. The relative amount each reporter signal calibrator can be based on the relative amount of each corresponding target protein fragment expected to be in the protein sample. The amount of each of the reporter signal calibrators can be the same.

In various embodiments of all of the aspects of the invention, the target protein fragments and reporter signal calibrators can be altered by fragmentation, or by cleavage at a photocleavable amino acid. The target protein fragments and reporter signal calibrators can be fragmented in a collision cell or at an asparagine-proline bond. The protein fragments can be produced by protease digestion of the protein sample. The protease may be a serine protease (e.g., trypsin). The protein fragments can be produced by digestion of the protein sample with Factor Xa or Enterokinase, or can be produced by cleavage at a photocleavable amino acid.

In various embodiments of all of the aspects of the invention, the common property can be mass-to-charge ratio, wherein the target protein fragments and reporter signal calibrators can be altered by altering their mass, their charge, or their mass and charge, wherein the altered forms of the target protein fragments and reporter signal calibrators can be distinguished via differences in the mass-to-charge ratio of the altered forms of the target protein fragments and reporter signal calibrators. The set of target protein fragments can comprise two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different target protein fragments. The set of target protein fragments can comprise ten or more different target protein fragments.

In various embodiments of all of the aspects of the invention, the set of reporter signal calibrators comprises two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signal calibrators. The reporter signal calibrators can comprise peptides, wherein the peptides have the same mass-to-charge ratio as the corresponding target protein fragments.

In various embodiments of all of the aspects of the invention, the peptides can have the same amino acid composition as the corresponding target protein fragments. The peptides can have the same amino acid sequence as the corresponding target protein fragments. Each peptide can have a different amino acid sequence than the corresponding target protein fragment. Each peptide can have a labile or scissile bond in a different location. The reporter signal calibrators can be peptides, oligonucleotides, carbohydrates, polymers, oligopeptides, or peptide nucleic acids.

In various embodiments of all of the aspects of the invention, the method can further comprise comparing the protein signature to one or more other protein signatures. At least one of the target protein fragments can comprise at least one modified amino acid. The modified amino acid can be a phosphorylated amino acid, an acylated amino acid, or a glycosylated amino acid. At least one of the target protein fragments can be the same as the target protein fragment comprising the modified amino acid except for the modified amino acid.

In various embodiments of all of the aspects of the invention, the method can further comprise performing steps (a) through (f) on a plurality of protein samples. The method can further comprise identifying differences between the protein signatures produced from the protein samples. The method can further comprise performing steps (a) through (f) on a control protein sample, identifying differences between the protein signatures produced from the protein samples and the control protein sample. The differences can be differences in the presence, amount, presence and amount, or absence of target protein fragments in the protein samples and the control protein sample.

In various embodiments of all of the aspects of the invention, the steps (a) through (f) can be performed on a control protein sample and a tester protein sample, wherein the tester protein sample, or the source of the tester protein sample, can be treated, prior to step (a), so as to destroy, disrupt or eliminate one or more protein molecules in the tester protein sample, wherein the target protein fragments corresponding to the destroyed, disrupted, or eliminated protein molecules will be produced from the control protein sample but not the tester protein sample. The tester protein sample can be treated so as to destroy, disrupt or eliminate one or more protein molecules in the tester protein sample. One or more protein molecules in the tester sample can be eliminated by separating the one or more protein molecules from the tester protein sample. One or more protein molecules can be separated by affinity separation. The source of the tester protein sample can be treated so as to destroy, disrupt or eliminate one or more protein molecules in the tester protein sample. The treatment of the source can be accomplished by exposing cells from which the tester sample will be derived with a compound, composition, or condition that will reduce or eliminate expression of one or more genes.

In various embodiments of all of the aspects of the invention, the method can further comprise identifying differences in the target protein fragments in the control protein sample and tester protein sample. The methods can further comprise identifying differences between the target protein fragments in the protein samples. The plurality of protein samples can be produced by a separation procedure, wherein the separation procedure can comprise liquid chromatography, gel electrophoresis, two-dimensional chromatography, two-dimensional gel electrophoresis, isoelectric focusing, thin layer chromatography, centrifugation, filtration, ion chromatography, immunoaffinity chromatography, membrane separation, or a combination of these. The protein samples can be different fractions or samples produced by the same separation procedure.

In various embodiments of all of the aspects of the invention, the method can further comprise performing steps (a) through (f) on a second protein sample. The second protein sample can be a sample from the same type of organism as the first protein sample. The second protein sample can be a sample from the same type of tissue as the first protein sample. The second protein sample can be a sample from the same organism as the first protein sample. The second protein sample can be obtained at a different time than the first protein sample. The second protein sample can be a sample from a different organism than the first protein sample. The second protein sample can be a sample from a different type of tissue than the first protein sample. The second protein sample can be a sample from a different species of organism than the first protein sample. The second protein sample can be a sample from a different strain of organism than the first protein sample. The second protein sample can be a sample from a different cellular compartment than the first protein sample.

In various embodiments of all of the aspects of the invention, the method can further comprise producing a second protein signature from a second protein sample and comparing the first protein signature and second protein signature, wherein differences in the first and second protein signatures indicate differences in source or condition of the source of the first and second protein samples. The method can further comprise producing a second protein signature from a second protein sample and comparing the first protein signature and second protein signature, wherein differences in the first and second protein signatures indicate differences in protein modification of the first and second protein samples.

In various embodiments of all of the aspects of the invention, the second protein sample can be a sample from the same type of cells as the first protein sample except that the cells from which the first protein sample is derived are modification-deficient relative to the cells from which the second protein sample is derived. The second protein sample can be a sample from a different type of cells than the first protein sample, and wherein the cells from which the first protein sample is derived are modification-deficient relative to the cells from which the second protein sample is derived. The protein sample can be derived from one or more cells. The protein signature can indicate the physiological state of the cells. The protein signature can indicate the effect of a treatment of the cells. The cells can be derived from an organism, wherein the cells can be treated by treating the organism. The organism can be treated by administering a compound to the organism. The organism can be human.

In various embodiments of all of the aspects of the invention, the protein sample can be produced by a separation procedure, wherein the separation procedure can comprise liquid chromatography, gel electrophoresis, two-dimensional chromatography, two-dimensional gel electrophoresis, isoelectric focusing, thin layer chromatography, centrifugation, filtration, ion chromatography, immunoaffinity chromatography, membrane separation, or a combination of these.

In various embodiments of all of the aspects of the invention, the set of reporter signal calibrators can consist of a single reporter signal calibrator. The protein signature of the protein sample can represent the presence, absence, amount, or presence and amount of the target protein fragment in the protein sample that corresponds to the reporter signal calibrator. The target protein fragments and reporter signal calibrators can be distinguished and/or separated from other molecules based on the common properties of the target protein fragments and reporter signal calibrators. The target protein fragments and reporter signal calibrators can be altered following separation. The target protein fragments can be produced by treating the protein sample. One or more of the indicator signal calibrators can generate a predetermined pattern under conditions that allow the target protein fragments to be separated from other protein fragments in the protein sample.

In various embodiments of all of the aspects of the invention, the method can further comprise determining the ratio of the amount of the target protein fragment and the amount of the reporter signal calibrator detected, and comparing the determined ratio with the predicted ratio of the amount of the target protein fragment and the amount of the reporter signal calibrator, wherein the predicted ratio can be based on the predicted amount of target protein fragment in the protein sample and the predetermined amount of reporter signal calibrator, wherein the predicted amount of target protein fragment is the amount of target protein fragment the protein sample would have if the known amount of protein in the protein sample consisted of the target protein (or target protein fragment), wherein the difference between the determined ratio and the predicted ratio is a measure of the purity of the protein sample for the target protein (or target protein fragment), wherein the closer the determined ratio is to the predicted ratio, the purer the protein sample. The reporter signal calibrator and one or more of the indicator signal calibrators can generate a predetermined pattern. The reporter signal calibrators can generate a predetermined pattern.

In various embodiments of all of the aspects of the invention, the method can further comprise, prior to or simultaneous with step (b), mixing the target protein fragments with a set of reporter signal calibrators, wherein each target protein fragment shares a common property with at least one of the reporter signal calibrators, wherein the common property allows the target protein fragments (e.g., each of these) and reporter signal calibrators having the common property to be distinguished and/or separated from molecules lacking the common property, wherein the reporter signal calibrators (e.g., each of these) can be altered, wherein the altered form of each reporter signal calibrator can be distinguished from the altered form of the target protein fragment with which the reporter signal calibrator shares a common property.

In a further aspect, the invention provides methods of detecting expression, the method comprising detecting a target altered reporter signal peptide derived from one or more expression samples, wherein the one or more expression samples collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the reporter signal peptides (or the amino acid segments comprising the reporter signal peptide) have a common property, wherein the common property allows the reporter signal peptides (or the amino acid segments) to be distinguished and/or separated from molecules lacking the common property, wherein the reporter signal peptides can be altered, wherein the altered form of each reporter signal peptide (or the amino acid segments) can be distinguished from the altered forms of the other reporter signal peptides (or the amino acid segments), wherein the target altered reporter signal peptide (or the tareget altered amino acid segments) is one of the altered reporter signal peptides (or one of the altered amino acid segments), wherein detection of the target altered reporter signal peptide (or the tareget altered amino acid segment) indicates expression of the amino acid segment that comprises the reporter signal peptide (or the nucleotide segment encoding the amino acid segment that comprises the reporter signal peptide) from which the target altered reporter signal peptide (or the targeted altered amino acid segment) is derived, wherein the reporter signal peptides (or the amino acid segments) and/or one or more of the indicator signal peptides will generate a predetermined pattern under conditions where the common property allows the reporter signal peptides to be distinguished and/or separated from molecules lacking the common property. In some embodiments, alteration of the reporter signal peptides alters the amino acid segments.

In a further aspect, the invention provides methods of detecting expression, the method comprising detecting an altered amino acid subsegment derived from one or more expression samples, wherein the one or more expression samples collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the amino acid segments each comprise an amino acid subsegment, wherein each amino acid subsegment comprises a portion of the protein or peptide of interest and all or a portion of the reporter signal peptide or indicator signal peptide, wherein the amino acid subsegments comprising all or a portion of the reporter signal peptide have a common property, wherein the common property allows the amino acid subsegments comprising all or a portion of the reporter signal peptide to be distinguished and/or separated from molecules lacking the common property, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates expression of the amino acid segment from which the target altered amino acid subsegment is derived, wherein the amino acid subsegments will generate a predetermined pattern under conditions where the common property allows the amino acid subsegments comprising all or a portion of the reporter signal peptide to be distinguished and/or separated from molecules lacking the common property.

In another aspect, the invention provides methods of detecting expression, the method comprising detecting a target altered reporter signal peptide derived from one or more expression samples, wherein the one or more expression samples collectively comprise a set of nucleic acid molecules, wherein each nucleic acid molecule comprises a nucleotide segment encoding an amino acid segment comprising a reporter signal peptide and a protein or peptide of interest, wherein the reporter signal peptides (or the amino acid segments) belong to one of two or more sets of reporter signal peptides (or the amino acid segments), wherein the reporter signal peptides (or the amino acid segments) in each set have a common property, wherein the common property in each set of reporter signal peptides (or the amino acid segments) is different from the common property in the other sets of reporter signal peptides (or the amino acid segments), wherein the common property allows the reporter signal peptides (or the amino acid segments) to be distinguished and/or separated from molecules lacking the common property, wherein the reporter signal peptides can be altered, wherein the altered form of each reporter signal peptide (or amino acid segment) can be distinguished from the altered forms of the other reporter signal peptides (or amino acid segments), wherein the target altered reporter signal peptide (or the target altered amino acid segment) is one of the altered reporter signal peptides (or one of the altered amino acid segments), wherein detection of the target altered reporter signal peptide (or the target altered amino acid segment) indicates expression of the amino acid segment that comprises the reporter signal peptide (or the nucleotide segment encoding the amino acid segment that comprises the reporter signal peptide) from which the target altered reporter signal peptide (or the target altered amino acid segment) is derived, wherein the reporter signal peptides (or amino acid segments) will generate a predetermined pattern under conditions where the common property allows the reporter signal peptides (or the amino acid segments comprising a reporter signal peptide) to be distinguished and/or separated from molecules lacking the common property. In some embodiments, alteration of the reporter signal peptides alters the amino acid segments.

In yet another aspect, the invention provides methods of detecting expression, the method comprising detecting an altered amino acid subsegment derived from one or more expression samples, wherein the one or more expression samples collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments belong to one of two or more sets of amino acid subsegments, wherein the amino acid subsegments in each set have a common property, wherein the common property in each set of amino acid subsegments is different from the common property in the other sets of amino acid subsegments, wherein the common property allows the amino acid subsegments comprising all or a portion of the reporter signal peptide to be distinguished and/or separated from molecules lacking the common property, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates expression of the amino acid segment from which the target altered amino acid subsegment is derived, wherein the amino acid subsegments will generate a predetermined pattern under conditions where the common property allows the amino acid subsegments comprising all or a portion of the reporter signal peptide to be distinguished and/or separated from molecules lacking the common property.

In an additional aspect, the invention provides methods of detecting cells or cell samples, the method comprising detecting a target altered reporter signal peptide derived from one or more cells or cell samples, wherein the one or more cells or the one or more cell samples collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the reporter signal peptides have a common property, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the target altered reporter signal peptide is one of the altered reporter signal peptides, wherein detection of the target altered reporter signal peptide indicates the presence of the cell or the cell sample from which the target altered reporter signal peptide is derived, wherein the reporter signal peptides and one or more of the indicator signal peptides will generate a predetermined pattern under conditions where the common property allows the reporter signal peptides to be distinguished and/or separated from molecules lacking the common property.

In a further aspect, the invention provides methods of detecting cells or cell samples, the method comprising detecting a target altered reporter signal peptide derived from one or more cells or cell samples, wherein the one or more cells or the one or more cell samples collectively comprise a set of nucleic acid molecules, wherein each nucleic acid molecule comprises a nucleotide segment encoding an amino acid segment comprising a reporter signal peptide and a protein or peptide of interest, wherein the reporter signal peptides belong to one of two or more sets of reporter signal peptides, wherein the reporter signal peptides in each set have a common property, wherein the common property in each set of reporter signal peptides is different from the common property in the other sets of reporter signal peptides, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the target altered reporter signal peptide is one of the altered reporter signal peptides, wherein detection of the target altered reporter signal peptide indicates the presence of the cell or the cell sample from which the target altered reporter signal peptide is derived, wherein the reporter signal peptides will generate a predetermined pattern under conditions where the common property allows the reporter signal peptides to be distinguished and/or separated from molecules lacking the common property.

In a further aspect, the invention provides methods of detecting cells or organisms, the method comprising detecting a target altered reporter signal peptide derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the reporter signal peptides have a common property, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the target altered reporter signal peptide is one of the altered reporter signal peptides, wherein detection of the target altered reporter signal peptide indicates the presence of the cell or organism from which the target altered reporter signal peptide is derived, wherein the reporter signal peptides and one or more of the indicator signal peptides will generate a predetermined pattern under conditions where the common property allows the reporter signal peptides to be distinguished and/or separated from molecules lacking the common property.

In another aspect, the invention provides methods of detecting cells or organisms, the method comprising detecting a target altered amino acid segment derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the amino acid segments comprising the reporter signal peptide have a common property, wherein the common property allows the amino acid segments comprising a reporter signal peptide to be distinguished and/or separated from molecules lacking the common property, wherein the reporter signal peptides can be altered, wherein alteration of the reporter signal peptides alters the amino acid segments, wherein the altered form of each amino acid segment can be distinguished from the altered forms of the other amino acid segments, wherein the target altered amino acid segment is one of the altered amino acid segments, wherein detection of the target altered amino acid segment indicates the presence of the cell or the organism from which the target altered amino acid segment is derived, wherein the amino acid segments will generate a predetermined pattern under conditions where the common property allows the amino acid segments comprising a reporter signal peptide to be distinguished and/or separated from molecules lacking the common property.

In a further aspect, the invention provides methods of detecting cells or organisms, the method comprising detecting an altered amino acid subsegment derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide or indicator signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments comprising all or a portion of the reporter signal peptide have a common property, wherein the common property allows the amino acid subsegments comprising all or a portion of the reporter signal peptide to be distinguished and/or separated from molecules lacking the common property, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates the presence of the cell or the organism from which the target altered amino acid subsegment is derived, wherein the amino acid subsegments will generate a predetermined pattern under conditions where the common property allows the amino acid subsegments comprising all or a portion of the reporter signal peptide to be distinguished and/or separated from molecules lacking the common property.

In a further aspect, the invention provides methods of detecting cells or organisms, the method comprising detecting a target altered reporter signal peptide derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide and a protein or peptide of interest, wherein the reporter signal peptides belong to one of two or more sets of reporter signal peptides, wherein the reporter signal peptides in each set have a common property, wherein the common property in each set of reporter signal peptides is different from the common property in the other sets of reporter signal peptides, wherein the common property allows the reporter signal peptides to be distinguished and/or separated from molecules lacking the common property, wherein the reporter signal peptides can be altered, wherein the altered form of each reporter signal peptide can be distinguished from the altered forms of the other reporter signal peptides, wherein the target altered reporter signal peptide is one of the altered reporter signal peptides, wherein detection of the target altered reporter signal peptide indicates the presence of the cell or the organism from which the target altered reporter signal peptide is derived, wherein the reporter signal peptides will generate a predetermined pattern under conditions where the common property allows the reporter signal peptides to be distinguished and/or separated from molecules lacking the common property.

In a further aspect, the invention provides methods of detecting cells or organisms, the method comprising detecting a target altered amino acid segment derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide and a protein or peptide of interest, wherein the amino acid segments belong to one of two or more sets of amino acid segments, wherein the amino acid segments in each set have a common property, wherein the common property in each set of amino acid segments is different from the common property in the other sets of amino acid segments, wherein the common property allows the amino acid segments to be distinguished and/or separated from molecules lacking the common property, wherein the reporter signal peptides can be altered, wherein alteration of the reporter signal peptides alters the amino acid segments, wherein the altered form of each amino acid segment can be distinguished from the altered forms of the other amino acid segments, wherein the target altered amino acid segment is one of the altered amino acid segments, wherein detection of the target altered amino acid segment indicates the presence of the cell or organism from which the target altered amino acid segment is derived, wherein the amino acid segments will generate a predetermined pattern under conditions where the common property allows the amino acid segments to be distinguished and/or separated from molecules lacking the common property.

In another aspect, the invention provides methods of detecting cells or organisms, the method comprising detecting an altered amino acid subsegment derived from one or more cells or organisms, wherein the one or more cells or the one or more organisms collectively comprise a set of nucleic acid molecules, wherein each nucleic acid molecule comprises a nucleotide segment encoding an amino acid segment comprising a reporter signal peptide and a protein or peptide of interest, wherein the amino acid segments each comprise an amino acid subsegment, wherein each amino acid subsegment comprises a portion of the protein or peptide of interest and all or a portion of the reporter signal peptide, wherein the amino acid subsegments belong to one of two or more sets of amino acid subsegments, wherein the amino acid subsegments in each set have a common property, wherein the common property in each set of amino acid subsegments is different from the common property in the other sets of amino acid subsegments, wherein the common property allows the amino acid subsegments comprising all or a portion of the reporter signal peptide to be distinguished and/or separated from molecules lacking the common property, wherein the reporter signal peptides can be altered, wherein alteration of the reporter signal peptides alters the amino acid subsegments, wherein the altered form of each amino acid subsegment can be distinguished from the altered forms of the other amino acid subsegments, wherein the target altered amino acid subsegment is one of the altered amino acid subsegments, wherein detection of the target altered amino acid subsegment indicates the presence of the cell or the organism from which the target altered amino acid subsegment is derived, wherein the amino acid subsegments will generate a predetermined pattern under conditions where the common property allows the amino acid subsegments comprising all or a portion of the reporter signal peptide to be distinguished and/or separated from molecules lacking the common property.

In various embodiments of all of the aspects of the invention, the method can further comprise determining the amount of the target altered reporter signal peptide detected, wherein the amount of the target altered reporter signal peptide indicates the amount present in the one or more expression samples of the amino acid segment that comprises the reporter signal peptide from which the target altered reporter signal peptide is derived. The amount of the amino acid segment present can be proportional to the amount of the target altered reporter signal peptide detected.

In various embodiments of all of the aspects of the invention, the method can further comprise detecting a plurality of the altered reporter signal peptides, wherein detection of each altered reporter signal peptide indicates expression of the amino acid segment that comprises the reporter signal peptide from which that altered reporter signal peptide is derived. The method can further comprise determining the amount of the altered reporter signal peptides detected, wherein the amount of each altered reporter signal peptide indicates the amount present in the one or more expression samples of the amino acid segment that comprises the reporter signal peptide from which that altered reporter signal peptide is derived. The amount of the amino acid segment present can be proportional to the amount of the altered reporter signal peptide detected.

In various embodiments of all of the aspects of the invention, the presence, absence, amount, or presence and amount of the altered forms of the reporter signal peptides can indicate the presence, absence, amount, or presence and amount in the expression sample of the reporter signal peptides from which the altered forms of the reporter signal peptides are derived, wherein the presence, absence, amount, or presence and amount of the reporter signal peptides in the expression sample constitutes a protein signature of the expression sample. The altered forms of the reporter signal peptides can be detected using mass spectrometry, such as by using a tandem mass spectrometer. The mass spectrometer can include a quadrupole set for single-ion filtering, a collision cell, and a time-of-flight spectrometer.

In various embodiments of all of the aspects of the invention, the reporter signal peptides can be altered by fragmentation or by cleavage at a photocleavable amino acid. The reporter signal peptides can be fragmented in a collision cell, and/or can be fragmented at an asparagine-proline bond, a methionine, or a phosphorylated amino acid. The common property can be mass-to-charge ratio, wherein the reporter signal peptides can be altered by altering their mass, their charge, or their mass and charge, wherein the altered forms of the reporter signal peptides can be distinguished via differences in the mass-to-charge ratio of the altered forms of the reporter signal peptides. The method can use two or more, three or more, four or more, five or more, six or more, seven or more, eight or more, nine or more, ten or more, twenty or more, thirty or more, forty or more, fifty or more, sixty or more, seventy or more, eighty or more, ninety or more, or one hundred or more different reporter signal peptides. Ten or more different reporter signal peptides can be used. Each peptide can have a labile or scissile bond in a different location.

In various embodiments of all of the aspects of the invention, the method can further comprise comparing the protein signature to one or more other protein signatures. The detected altered reporter signal peptides can be derived from a plurality of expression samples. Some of the detected altered reporter signal peptides can be derived from a control expression sample. The method can further comprise identifying differences between the protein signatures produced from the expression samples and the control expression sample. The differences can be differences in the presence, amount, presence and amount, or absence of reporter signal peptides in the expression samples and the control expression sample. The plurality of expression samples can comprise a control expression sample and a tester expression sample, wherein the tester expression sample, or the source of the tester expression sample, can be treated so as to destroy, disrupt or eliminate one or more of the amino acid segments in the tester expression sample, wherein the reporter signal peptides corresponding to the destroyed, disrupted, or eliminated amino acid segments will be produced from the control expression sample but not the tester expression sample.

In various embodiments of all of the aspects of the invention, the tester expression sample can be treated so as to destroy, disrupt or eliminate one or more of the amino acid segments in the tester expression sample. One or more of the amino acid segments in the tester sample can be eliminated by separating the one or more of the amino acid segments from the tester expression sample. One or more of the amino acid segments can be separated by affinity separation. The source of the tester expression sample can be treated so as to destroy, disrupt or eliminate one or more of the amino acid segments in the tester expression sample. The treatment of the source can be accomplished by exposing cells from which the tester sample will be derived with a compound, composition, or condition that will reduce or eliminate expression of one or more of the nucleotide segments.

In various embodiments of all of the aspects of the invention, the method can further comprise identifying differences in the reporter signal peptides in the control expression sample and tester expression sample. The method can further comprise identifying differences between the reporter signal peptides in the expression samples. At least two of the expression samples, or the sources of the at least two expression samples, can be subjected to different conditions. The sources of the expression samples can be cells. Differences in the protein signatures of the at least two expression samples can indicate the effect of the different conditions. The different conditions can be exposure to different compounds. The different conditions can be exposure to a compound and no exposure to the compound.

In various embodiments of all of the aspects of the invention, the method can further comprise producing a second protein signature from a second expression sample and comparing the first protein signature and second protein signature, wherein differences in the first and second protein signatures indicate differences in source or condition of the source of the first and second expression samples. The method can further comprise producing a second protein signature from a second expression sample and comparing the first protein signature and second protein signature, wherein differences in the first and second protein signatures indicate differences in protein modification of the first and second expression samples. The second expression sample can be a sample from the same type of cells as the first expression sample except that the cells from which the first expression sample is derived are modification-deficient relative to the cells from which the second expression sample is derived. The second expression sample can be a sample from a different type of cells than the first expression sample, and wherein the cells from which the first expression sample is derived are modification-deficient relative to the cells from which the second expression sample is derived.

In various embodiments of all of the aspects of the invention, the expression sample can be derived from one or more cells. The protein signature can indicate the physiological state of the cells, or can indicate the effect of a treatment of the cells. The cells can be derived from an organism, wherein the cells can be treated by treating the organism. The organism can be treated by administering a compound to the organism. The organism can be human.

In various embodiments of all of the aspects of the invention, it will be understood that altered reporter signal peptides can be detected in a first and a second expression sample. The second expression sample can be a sample from the same organism, a different organism, a different species of organism, a different strain of organism, or the same type of organism as the first expression sample. The second expression sample can be a sample from the same type of tissue as the first expression sample. The second expression sample can be obtained at a different time than the first expression sample. The second expression sample can be a sample from a different type of tissue or from a different cellular compartment than the first expression sample.

In various embodiments of all of the aspects of the invention, the method can further comprise altering the reporter signal peptides. The reporter signal peptides can be altered by fragmentation or by cleavage at a photocleavable amino acid. The reporter signal peptides can be fragmented in a collision cell. The reporter signal peptides can be fragmented at an asparagine-proline bond, a methionine, or a phosphorylated amino acid.

In various embodiments of all of the aspects of the invention, the method can further comprise separating the reporter signal peptides from the expression samples. The reporter signal peptides can be distinguished and/or separated from the expression samples based on the common property. The method can further comprise cleaving the reporter signal peptides from the proteins or peptides of interest. The reporter signal peptides can be distinguished and/or separated from the proteins or peptides of interest based on the common property. The method can further comprise cleaving the amino acid segments into a reporter signal peptide portion and a protein portion. The method can further comprise mixing two or more of the expression samples together.

In various embodiments of all of the aspects of the invention, the method can further comprise mixing two or more amino acid segments together, wherein the mixed amino acid segments were derived from two or more different expression samples. Expression of the amino acid segment that comprises the reporter signal peptide from which the target altered reporter signal peptide is derived can identify the expression sample from which the target altered reporter signal peptide is derived. The expression samples can be derived from one or more cells, wherein expression of the amino acid segment that comprises the reporter signal peptide from which the target altered reporter signal peptide is derived identifies the cell from which the identified expression sample is derived. The expression samples can be derived from one or more organisms, wherein expression of the amino acid segment that comprises the reporter signal peptide from which the target altered reporter signal peptide is derived identifies the organism from which the identified expression sample is derived. The expression samples can be derived from one or more tissues, wherein expression of the amino acid segment that comprises the reporter signal peptide from which the target altered reporter signal peptide is derived identifies the tissue from which the identified expression sample is derived.

In various embodiments of all of the aspects of the invention, the expression samples can be derived from one or more cell lines, wherein expression of the amino acid segment that comprises the reporter signal peptide from which the target altered reporter signal peptide is derived identifies the cell line from which the identified expression sample is derived. Each nucleic acid molecule can further comprise expression sequences, wherein the expression sequences can be operably linked to the nucleotide segment such that the amino acid segment is expressed. The expression sequences can comprise translation expression sequences and/or transcription expression sequences. The amino acid segment can be expressed in vitro or in vivo. The amino acid segment can be expressed in cell culture. The expression sequences of each nucleic acid molecule can be different. The different expression sequences can be differently regulated. The expression sequences can be similarly regulated.

In various embodiments of all of the aspects of the invention, it will be understood that a plurality of the expression sequences can be expression sequences of, or derived from, genes expressed as part of the same expression cascade. The expression sequences of each nucleic acid molecule can be the same or can be similarly regulated. The expression sequences of at least two nucleic acid molecules can be different or can be the same. Expression of the amino acid segment can be induced. Each nucleic acid molecule can further comprise replication sequences, wherein the replication sequences mediate replication of the nucleic acid molecules. The nucleic acid molecules can be replicated in vitro or in vivo. The nucleic acid molecules can be replicated in cell culture. Each nucleic acid molecule further can comprise integration sequences, wherein the integration sequences mediate integration of the nucleic acid molecules into other nucleic acids. The nucleic acid molecules can be integrated into a chromosome (e.g., at a predetermined location).

In various embodiments of all of the aspects of the invention, the nucleic acids molecules can be produced by replicating nucleic acids in one or more nucleic acid samples. The nucleic acids can be replicated using pairs of primers, wherein each of the first primers in the primer pairs used to produce the nucleic acid molecules comprises a nucleotide sequence encoding the reporter signal peptide. Each first primer further comprises expression sequences. The nucleotide sequence of each first primer also can encode an epitope tag. Each amino acid segment can further comprise an epitope tag. The epitope tag of each amino acid segment can be different or can be the same. The epitope tag of at least two amino acid segments can be different or can be the same. The amino acid segments can be distinguished and/or separated from the one or more expression samples via the epitope tags.

In various embodiments of all of the aspects of the invention, the reporter signal peptide of each amino acid segment can be different or can be the same. The reporter signal peptide of at least two amino acid segments can be different or can be the same. The nucleic acid molecules can be in cells or cell lines. Each nucleic acid molecule can be in a different cell (or cell line) or can be in the same cell (or cell line). Each nucleic acid molecule can further comprise expression sequences, wherein the expression sequences can be operably linked to the nucleotide segment such that the amino acid segment can be expressed. The expression sequences of each nucleic acid molecule can be different or can be similarly regulated. A plurality of the expression sequences can be expression sequences of, or derived from, genes expressed as part of the same expression cascade.

In various embodiments of all of the aspects of the invention, the nucleic acid molecules can be integrated into a chromosome of the cell (or cell line). The nucleic acid molecules can be integrated into the chromosome at a predetermined location. The chromosome can be an artificial chromosome. The nucleic acid molecules can be, or can be integrated into, a plasmid. The cells can be in cell lines. Each nucleic acid molecule can be in a different cell or cell line or can be in the same cell or cell line. The expression samples can be produced from the cells. Each expression sample can be produced from cells from a cell sample, wherein each expression sample can be produced from a different cell sample. Each cell sample can be subjected to different conditions, brought into contact with a different test compound, cultured under different conditions, derived from a different organism, derived from a different tissue, or taken from the same source at different times. The expression samples can be produced by lysing the cells.

In various embodiments of all of the aspects of the invention, the nucleic acid molecules can be in organisms. Each nucleic acid molecule can be in a different organism or can be in the same organism. Each nucleic acid molecule can further comprise expression sequences, wherein the expression sequences can be operably linked to the nucleotide segment such that the amino acid segment can be expressed. The expression sequences of each nucleic acid molecule can be different or can be similarly regulated. A plurality of the expression sequences can be expression sequences of, or derived from, genes expressed as part of the same expression cascade. The nucleic acid molecules can be integrated into a chromosome of the organism (e.g., integrated into the chromosome at a predetermined location). The chromosome can be an artificial chromosome. The nucleic acid molecules can be, or can be integrated into, a plasmid. Each nucleic acid molecule can be in a different organism or can be in the same organism. The nucleic acid molecules can be in cells of an organism (e.g., in substantially all of the cells of the organism or in some of the cells of the organism). The amino acid segments can be expressed in substantially all of the cells of the organism or in some of the cells of the organism.

In various embodiments of all of the aspects of the invention, the protein or peptide of interest of each amino acid segment can be different or can be the same. The protein or peptide of interest of at least two amino acid segments can be different or can be the same. The proteins or peptides of interest can be related, can be proteins produced in the same cascade, can be proteins in the same enzymatic pathway, can be proteins expressed under the same conditions, can be proteins associated with the same disease, or can be proteins associated with the same cell type or the same tissue type.

In various embodiments of all of the aspects of the invention, the nucleotide segment can encode a plurality of amino acid segments each comprising a reporter signal peptide or indicator signal peptide and a protein or peptide of interest. The protein or peptide of interest of at least two of the amino acid segments in one of the nucleotide segments can be different. The protein or peptide of interest of the amino acid segments in one of the nucleotide segments can be different. The protein or peptide of interest of at least two of the amino acid segments in each of the nucleotide segments can be different. The protein or peptide of interest of the amino acid segments in each of the nucleotide segments can be different.

In various embodiments of all of the aspects of the invention, the set can consist of a single nucleic acid molecule. The set can consist of a single nucleic acid molecule, wherein the nucleic acid molecule comprises a plurality of nucleotide segments each encoding an amino acid segment. The amino acid segment can comprise a cleavage site near the junction between the reporter signal peptide and the protein or peptide of interest. The cleavage site can be cleaved. The reporter signal peptide can be distinguished and/or separated from the peptide or protein of interest. The cleavage site can be a trypsin cleavage site. The cleavage site can be at the junction between the reporter signal peptide and the protein or peptide of interest. Each amino acid segment can further comprise a self-cleaving segment. The self-cleaving segment can be between the reporter signal peptide and the protein or peptide of interest. The self-cleaving segment can cleave the amino acid segment. The reporter signal peptide can be distinguished and/or separated from the peptide or protein of interest. The self-cleaving segment can be an intein segment.

In various embodiments of all of the aspects of the invention, it will be understood that a plurality of different altered reporter signal peptides can be detected, wherein detection of each altered reporter signal peptide indicates either the expression of the amino acid segment that comprises the reporter signal peptide from which that altered reporter signal peptide is derived or the presence of the cell sample from which that altered reporter signal peptide is derived. Different expression samples or cell samples can comprise different nucleic acid molecules, wherein detection of each altered reporter signal peptide indicates either the expression in the expression sample that comprises the nucleic acid molecule that comprises the nucleotide segment encoding the amino acid segment that comprises the reporter signal peptide from which that altered reporter signal peptide is derived or the presence of the cell sample that comprises the nucleic acid molecule that comprises the nucleotide segment encoding the amino acid segment that comprises the reporter signal peptide from which that altered reporter signal peptide is derived.

In various embodiments of all of the aspects of the invention, it will be understood that a plurality of different expression samples can be used, wherein each different expression sample comprises different nucleic acid molecules, wherein detection of an altered reporter signal peptide indicates expression in the expression sample that comprises the nucleic acid molecule that comprises the nucleotide segment encoding the amino acid segment that comprises the reporter signal peptide from which the detected altered reporter signal peptide is derived.

In various embodiments of all of the aspects of the invention, each cell or organism can be engineered to contain at least one of the nucleic acid molecules, wherein the reporter signal peptide of the amino acid segment encoded by the nucleotide segment of the nucleic acid molecule in each cell or organism can be different. Each cell having a trait of interest can comprise the same reporter signal peptide, and organism having a trait of interest can comprise the same reporter signal peptide. The trait of interest can be a heterologous gene or a transgene. The heterologous gene or transgene can comprise the nucleic acid molecule. The heterologous gene or transgene can encode the amino acid segment. A plurality of different altered reporter signal peptides can be detected, wherein detection of each altered reporter signal peptide indicates the presence of the cell from which that altered reporter signal peptide is derived.

In various embodiments of all of the aspects of the invention, it will be understood that different cells or organisms can comprise different nucleic acid molecules, wherein detection of each altered reporter signal peptide indicates the presence of the cell or organism that comprises the nucleic acid molecule that comprises the nucleotide segment encoding the amino acid segment that comprises the reporter signal peptide from which that altered reporter signal peptide is derived.

In various embodiments of all of the aspects of the invention, it will be understood that a plurality of different cells, cell samples, or organisms can be used, wherein each different cell, cell sample or organism comprises different nucleic acid molecules, wherein detection of an altered reporter signal peptide indicates the presence of the cell, cell sample or organism that comprises the nucleic acid molecule that comprises the nucleotide segment encoding the amino acid segment that comprises the reporter signal peptide from which the detected altered reporter signal peptide is derived.

In a further aspect, the invention provides methods of detecting analytes, the method comprising associating one or more detectors with one or more target samples, wherein the detectors each comprise a specific binding molecule, a carrier, and a block group, wherein the block group comprises blocks, wherein the blocks comprise a set of reporter signals and one or more indicator signals (and/or two or more sets of reporter signals), and detecting the block group. The reporter signals in each set can have a common property, wherein the common property can allow the reporter signals to be distinguished or separated from molecules lacking the common property, wherein the reporter signals can be altered, wherein the altered forms of each reporter signal can be distinguished from every other altered form of reporter signal. The reporter signals and one or more of the indicator signals (or two or more of the sets of reporter signals) will generate a predetermined pattern under conditions where the common property allows the reporter signals to be distinguished and/or separated from molecules lacking the common property. In some forms, the indicator signals do not have the common property. The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. Alteration of the reporter signals also can alter their charge.

In various embodiments of all of the aspects of the invention, the detectors can be associated with one or more analytes and detected. Such detection can comprise, for example, (a) separating a set of reporter signals and one or more indicator signals (and/or two or more sets of reporter signals), where each reporter signal has a common property, from molecules lacking the common property, (b) identifying a predetermined pattern generated by the reporter signals and one or more of the indicator signals (and/or generated by the two or more sets of reporter signals), (c) altering the reporter signals that generate the predetermined pattern, (d) detecting and distinguishing the altered forms the reporter signals from each other.

Thus, it is an object of the present invention to provide a method for the multiplexed determination of presence, amount, or presence and amount of analytes. It is another object of the present invention to provide labeled proteins such that the presence, amount, or presence and amount of the proteins can be determined. It is another object of the present invention to provide a method for labeling proteins so as to allow the multiplexed determination of presence, amount, or presence and amount of proteins. It is another object of the present invention to provide a method for the multiplexed determination of presence, amount, or presence and amount of proteins. It is an object of the present invention to provide a method for detecting a mass tag signature. It is an object of the present invention to provide a method for detecting a protein signature. It is another object of the present invention to provide an assessment of the identity and purity of the peptides comprising a protein signature. It is another object of the present invention to provide a method for detecting phosphopeptides, or other posttranslational protein modifications, among the peptides comprising a protein signature. It is another object of the present invention to provide kits for generating mass tag signatures. It is another object of the present invention to provide kits for generating protein signatures.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a diagram of an example of the use of multidimension signals. Two samples are labeled independently with two sets of multidimension signals (Label Set 1 and Label Set 2). The labeled samples are mixed, subjected to trypsin digestion (this will cleave proteins in the samples). The mixed, trypsinized sample is cleaned up with HPLC and then subjected to two rounds of mass spectrometry. Example 1 provides an example of an assay following the steps shown in FIG. 1.

FIGS. 2A and 2B are graphs of mass spectrometry spectra of bovine serum albumin fragments labeled with multidimension signals. FIG. 2A covers m/z 1200 to 2500. FIG. 2B covers m/z from 500 to 1200. These spectra represent an example of an indicator level of analysis in the disclosed methods in which predetermined patterns are to be identified. FIG. 2A is from MALDI QSTAR instrument. The doublets spaced by 18 Dalton correspond to the mass difference between members of Label Set 1 (heavy) and Label Set 2 (light) shown in Table 3. The pair near m/z=1360 are spaced apart by 36 Dalton, corresponding to a peptide with two cysteines and thus two multidimension signals. The presence of two multidimension signals doubles the mass difference between the fragment labeled with a member of Label Set 1 and a member of Label Set 2. FIG. 2B is from ESI LTQ FTMS. The doublets are spaced apart by 18 Dalton correspond to the mass difference between members of Label Set 1 (heavy) and Label Set 2 (light) shown in Table 3. These doublets (spaced at multiples of 18 Daltons) represent a predetermined pattern expected from the use of multidimension labels in Label Set 1 and Label Set 2. Example 1 descibes the generation of the graphs in FIGS. 2A and 2B.

FIGS. 3A and 3B are graphs of mass spectrometry spectra of bovine serum albumin fragments labeled with multidimension signals. These spectra represent an example of a reporter level of analysis in the disclosed methods in which portions of a sample identified by predetermined patterns are subjected to further analysis (MS/MS in this case). FIG. 3A is a MS/MS spectrum of the peak at m/z 898.44 shown in FIG. 2B (lighter peak of the doublet). This peak represents a portion of the sample analyzed in FIG. 2B identified for the further analysis shown in FIG. 3A based on a predetermined pattern (peak doublets spaced at multiples of 18 Daltons). This peak represents protein fragments labeled with multidimension signals from Label Set 2 (the lighter set; see Table 3). The multidimension signals fragment at the D-P residues in the signals to produce pairs of fragments of characteristic mass. The two sets of 5 peaks in FIG. 3A represent pairs of fragments that result from fragmentation of the multidimension signals (one peak from one set of peaks paired with a peak from the other set). The peaks in a set of 5 peaks are separated by about 57 Daltons.

FIG. 3B is a MS/MS spectrum of the peak at m/z 907.45 shown in FIG. 2B (heavier peak of the doublet). This peak represents a portion of the sample analyzed in FIG. 2B identified for the further analysis shown in FIG. 3B based on a predetermined pattern (peak doublets spaced at multiples of 18 Daltons). This peak represents protein fragments labeled with multidimension signals from Label Set 1 (the heavier set; see Table 3). The multidimension signals fragment at the D-P residues in the signals to produce pairs of fragments of characteristic mass. The two sets of 7 peaks in FIG. 3B (which are tightly spaced in the graph) represent pairs of fragments that result from fragmentation of the multidimension signals (one peak from one set of peaks paired with a peak from the other set). The peaks in a set of 7 peaks are separated by about 3 Daltons (which is not well resolved at the resolution of the graph). Example 1 describes the generation of the graphs in FIGS. 3A and 3B.

FIGS. 4A and 4B are diagrams of examples of the logical flow of examples of the disclosed methods. In FIG. 4A, a mass spectrometry spectrum is collected (first box), and the spectrum is analyzed to detect a non-isobaric patterns (second box). The first two boxes correspond to an indicator level of analysis. The spectrum can be scanned for predetermined patterns (first circle). If a predetermined pattern is not detected, the indicator level of analysis is repeated for another sample or portion of sample (loop from first circle to first box). If a predetermined pattern is detected, a portion of the sample where the pattern was detected is sent for another level of analysis (first circle; downward arrow). A tandem mass spectrometry spectrum is collected on the portion of the sample (third box), and the spectrum is analyzed for information about the sample. The third and fourth boxes correspond to a reporter signal level of analysis. The entire analysis can be repeat on additional samples (loop from second circle to first box).

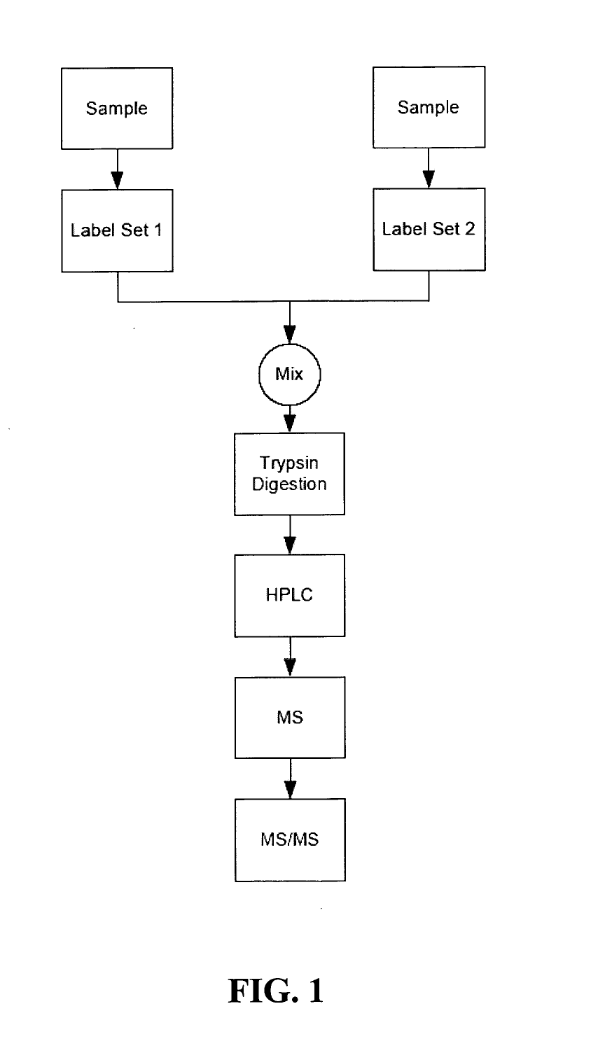

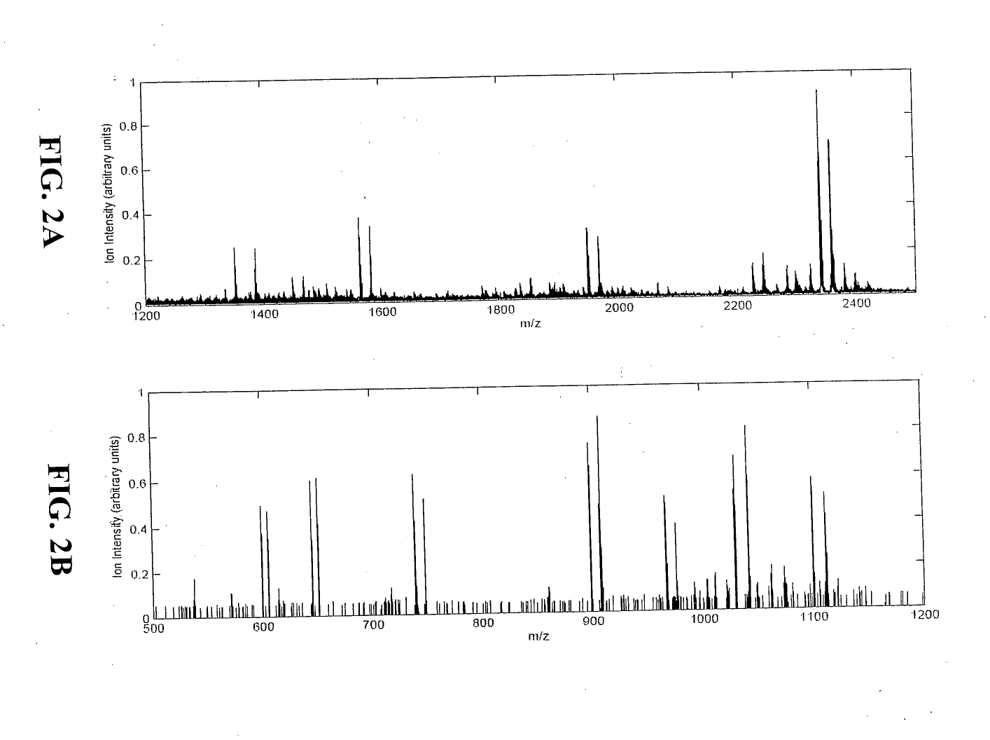

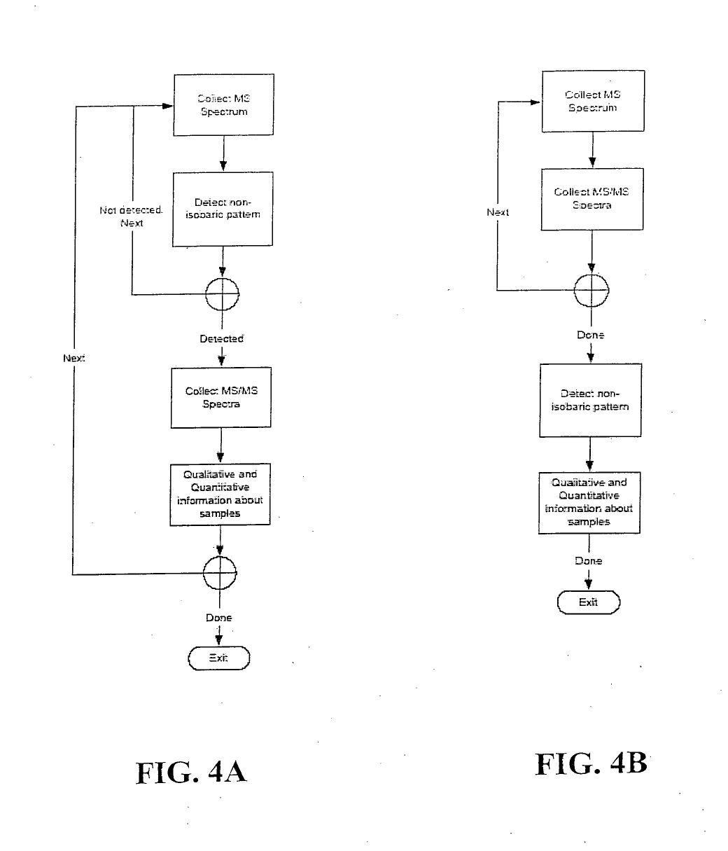

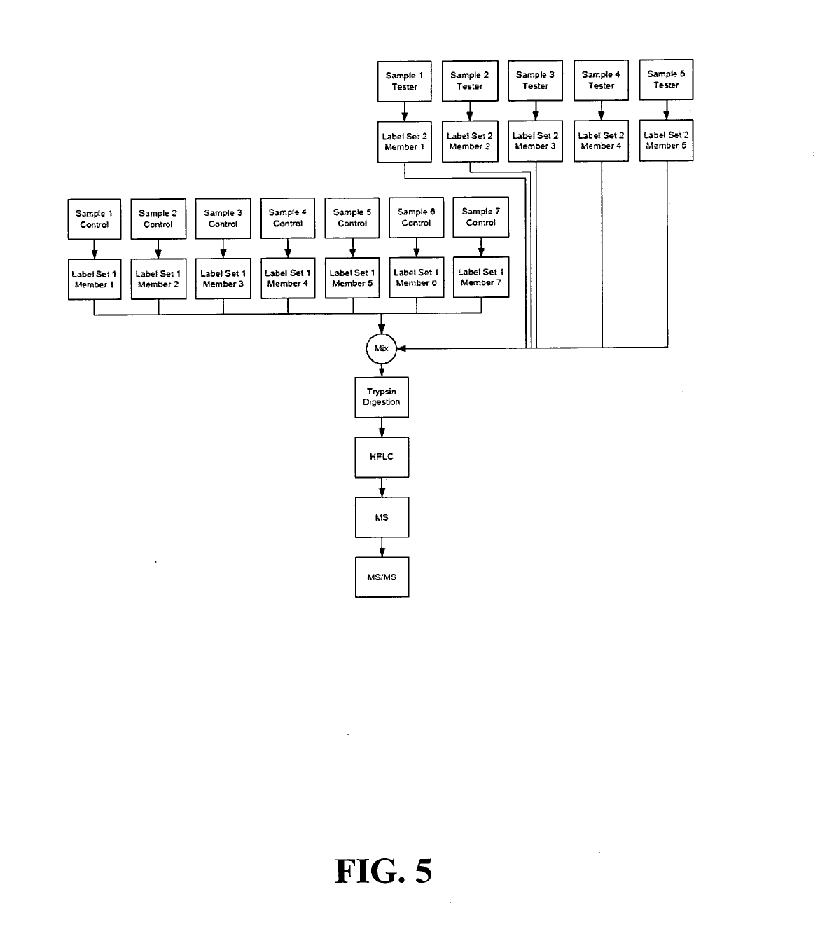

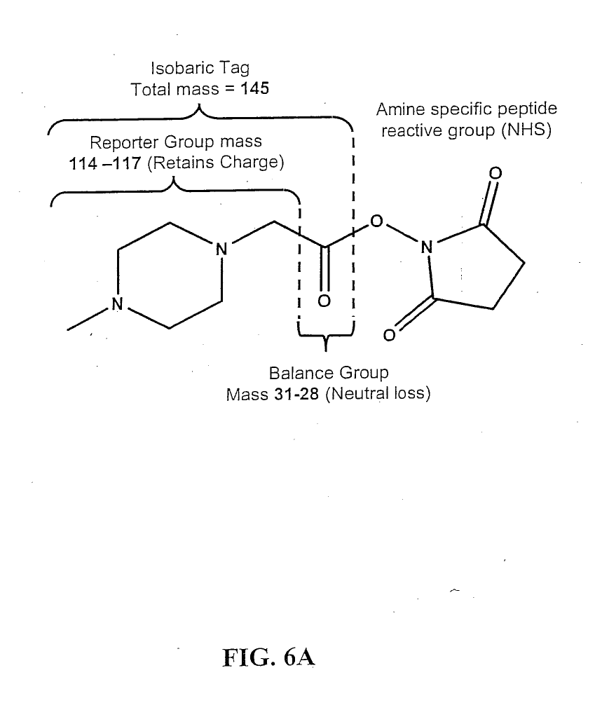

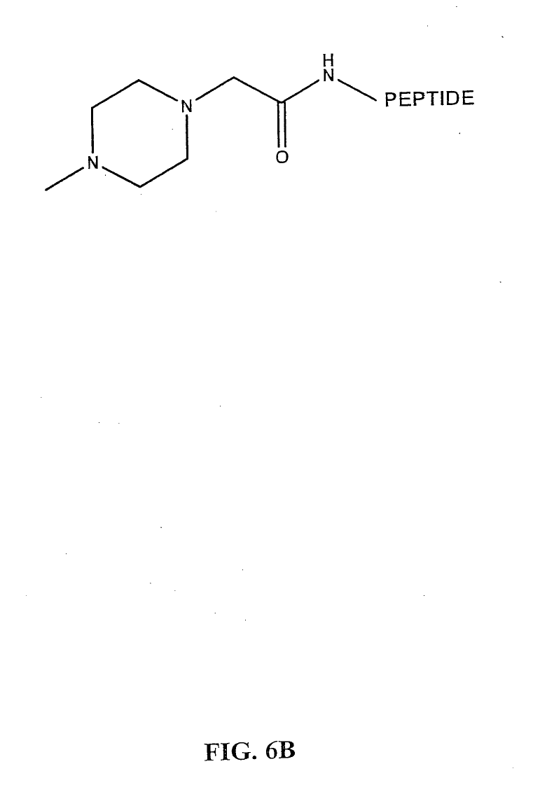

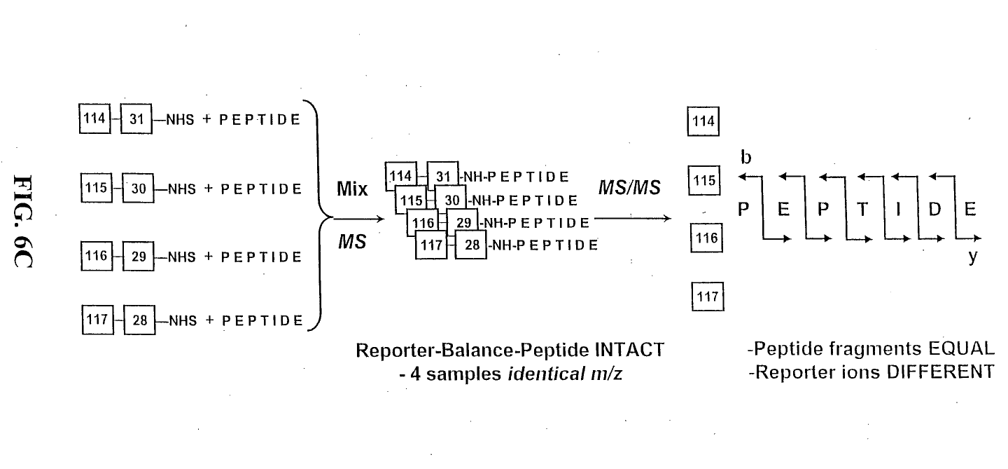

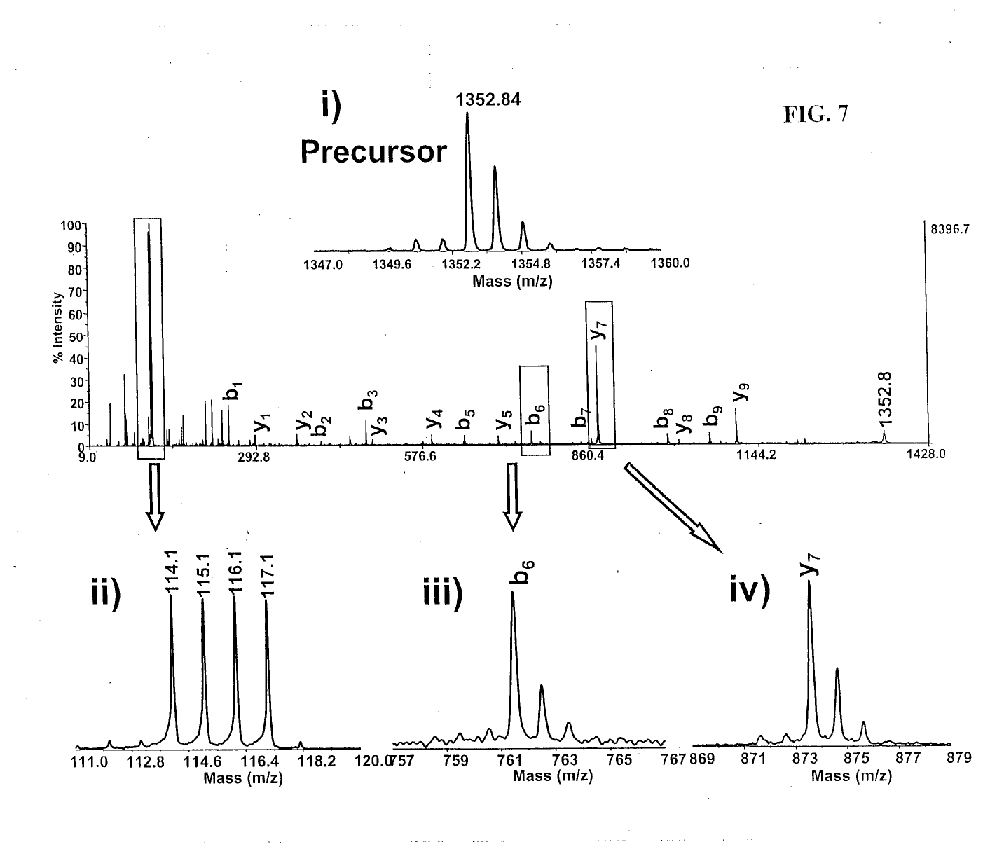

In FIG. 4B, a mass spectrometry spectrum is collected (first box). A tandem mass spectrometry spectrum is collected on the portion of the sample (second box). The first two stages can be repeated on additional samples (loop from first circle to first box). The spectrums are analyzed to detect a non-isobaric patterns (third box), and the spectrum is analyzed for information about the sample (fourth box). The first and third boxes correspond to an indicator level of analysis. The second and fourth boxes correspond to a reporter signal level of analysis. FIG. 4B is an example of separation of the data gathering and data analysis parts of the levels of analysis in the disclosed methods. FIG. 4 is an example of the analysis that can be involved in and between the two mass spectrometry stages shown in FIG. 1. Example 1 provides an example use of the logical flow shown in FIG. 4.

FIG. 5 is a diagram of an example of the use of multidimension signals. FIG. 5 is an example of the method shown in FIG. 1 where two different sample sets (Control samples and Tester samples) are labeled with different members of two different sets of multidimension signals (Label Set 1 and Label Set 2). In this example, 5 different Tester samples are each labeled with a different member of Label Set 2 and 7 different Control samples are each labeled with a different member of Label Set 1. The label sets can be, for example, the label sets shown in Table 3. The correlation between the label sets and the Control and Tester samples is for clarity and does not represent a limitation of the method. The labeled samples are mixed, subjected to trypsin digestion (this will cleave proteins in the samples). The mixed, trypsinized sample is cleaned up with HPLC and then subjected to two rounds of mass spectrometry. A preferred form of the method mixes labeled Control and Tester samples across Label Set 1 and Label Set 2.

FIGS. 6A, 6B and 6C are diagrams of the structure of iTRAQ multiplexed isobaric tagging chemistry. FIG. 6A shows the complete molecule consists of a reporter group (based on N-methylpiperazine) a mass balance group (carbonyl) and a peptide reactive group (NHS ester). The reporter group ranges in mass from m/z 114.1 to 117.1, while the balance group ranges in mass from 28 to 31 Da, such that the combined mass remains constant (145.1 Da) for each of the 4 reagents. FIG. 6B shows the structure when the tag is reacted with a peptide and forms an amide linkage to a peptide amine (N-terminal or epsilon amino group of lysine). FIG. 6C illustrates the isotopic tagging used to arrive at 4 isobaric combinations with 4 different reporter group masses (left). A mixture of 4 identical peptides each labeled with one member of the multiplex set appears as a single, unresolved precursor ion in MS (identical m/z; middle). Following collision induced dissociation (CID), the 4 reporter group ions appear as distinct masses (114-117 Da; right).

FIG. 7 shows an example of an MS/MS spectrum of the peptide TPHPALTEAK from a protein digest mixture prepared by labeling 4 separate digests with each of the 4 isobaric reagents and combining the reaction mixtures in a 1:1:1:1 ratio. The isotopic distribution of the precursor ([M+H]+, m/z 1352.84) is shown in i). Boxed components of the spectrum shown in the middle are shown on the bottom. These are a low mass region showing the signature ions used for quantitation in ii), isotopic distribution of the b₆ fragment in iii), and isotopic distribution of the Y₇ fragment ion in iv). The peptide is labeled by isobaric tags at both the N-terminus and C-terminal lysine side-chain. The precursor ion and all the internal fragment ions (e.g. type b- and y-) therefore contain all four members of the tag set, but remain isobaric. The example shown is the spectrum obtained from the singly-charged [M+H]+ peptide using a 4700 MALDI TOF-TOF analyzer.

### DETAILED DESCRIPTION OF THE INVENTION

Current technologies are limited in their ability to multiplex labels. In contrast, the disclosed methods of the invention allow the readout of many samples simultaneously and high internal accuracy in comparison to a sequential readout system. The disclosed methods have advantageous properties which can be used as a detection system in a number of fields, including antibody or protein microarrays, DNA microarrays, expression profiling, comparative genomics, immunology, diagnostic assays, and quality control.

The disclosed method and compositions may be understood more readily by reference to the following detailed description of particular embodiments and the Example included therein and to the Figures and their previous and following description.

Disclosed are compositions and methods for sensitive detection of one or multiple analytes (including proteins). In general, the methods involve the use of special label components, referred to as multidimension signals (MDS). In the disclosed methods, analysis of multidimension signals can result in one or more predetermined patterns that serve to indicate whether a further level of analysis can or should be performed and/or which portion(s) of the analyzed material can or should be analyzed in a further level of analysis. This is useful because multiple levels of analysis can be time consuming and generate large amounts of data and use of predetermined patterns in one level of analysis to indicate whether and on what portion(s) a further analysis should be based can limit the amount of work and focus data collection on material of interest. A useful example of analysis is mass spectrometry and a useful example of a predetermined pattern is a pattern of mass spectrometry peaks based on mass-to-charge ratios.

Analysis of isobaric reporter signals by mass spectrometry generally requires two rounds of mass spectrometry, the first to select material of a given mass-to-charge ratio (which corresponds to the mass-to charge ratio of the isobaric reporter signals of interest) and the second to detect and identify the different forms of altered reporter signals. In samples involving numerous different analytes labeled with reporter signals, each differentanalyte might require separate selection in the first round of mass spectrometry and separate detection of altered forms of reporter signal for each analyte. This could be very time consuming. Even if separation and detection could be accomplished simultaneously for multiple labeled analytes, this would generate enormous amounts of data because portions of the sample that collectively cover the entire range of mass-to-charge ratios would need to be subjected to the second round of mass spectrometry separately in order to identify the reporter signals present and associate them with different analytes. The disclosed method solves this problem by providing a means of identifying which samples and which portions of those samples should be further analyzed in a next level of analysis.

Preferred forms of the disclosed methods combine the use of isobaric technology with non-isobaric technologies to yield a system with improved workflow characteristics. In this workflow, with a mass spectrometric readout, scanning the non-isobaric labels in the MS dimension (indicator level of analysis) to trigger MS/MS events on the isobaric labels (reporter signal level of analysis) provides for an efficient data collection system.

The disclosed methods can make use of any suitable isobaric labeling system such as i-PROT (described in U.S. Application No. 2003/0194717, U.S. Application No. 2004/0220412, U.S. Application No. 2003/0124595, and U.S. Pat. No. 6,824,981), iTRAQ (described in U.S. Application No. 2004/0220412, and in PCT Application No. WO2004/070352), TMT (described in U.S. Application No. 2003/0194717), and the isobaric systems disclosed herein are examples, which provide enhanced data quality through their multiplexed MS/MS readout and the property that they do not increase the complexity of an MS spectrum. Such isobaric labeling systems can be combined or multiplexed using the principles disclosed herein to create non-isobaric relationships between the isobaric labeling systems. Alternatively or in addition, the disclosed methods can also make use of any suitable non-isobaric labels such as ICAT labels (described in PCT Application No. WO00/011208, and examples of using ICAT labels are described in PCT Application No. WO02/090929 and U.S. Application No. 2002/0192720), mass defect tags (such as those described in U.S. Application No. 2002/0172961 and Hall et al., J. Mass Spectrometry 38:809-816 (2003), other labels such as the labels described in U.S. Application Nos. 2004/0018565, 2003/0100018, 2003/0050453, 2004/0023274, 2002/014673, 2003/0022225, U.S. Pat. Nos. 6,312,893, 6,312,904, 6,629,040, Geysen et al., Chemistry & Biology 3(8):679-688 (1996), and the non-isobaric systems disclosed herein are examples. When attached to an analyte of interest, non-isobaric labels may be distinguished by MS, whereas isobaric labels require MS/MS or higher order (the order depending on the level on convolution of the isobaric labels and the manner of analysis). That is, isobaric MS species can be resolved by MS/MS; isobaric MS/MS species can be resolved by MS/MS/MS, and so forth. The time required to collect higher order spectra is generally longer than lower order spectra. The disclosed methods use the lower order spectra to trigger the more costly higher order data collection (thus making use of higher order data collection more sparingly and efficiently). Additionally, the amount of data storage increases quickly with higher order spectra, and such a triggering system allows for storage of only the data for those species of interest. Also, downstream data mining can be more efficient if only pertinent data is passed through.

In the disclosed multilevel analysis, an analysis level that can generate one or more predetermined patterns which can then serve as an indicator that another level or dimension of analysis can be performed and/or that serves as an indicator that portion(s) of the analysis sample should be analyzed in the next level of analysis can be referred to as an indicator level, indicator analysis or indicator level of analysis. Some forms of multilevel analyses can be performed where one of the levels of analysis is an indicator level. In this way, the disclosed indicator levels of analysis can be combined with any other technique or method of processing or analysis of samples and analytes, either before or following the indicator analysis.

A given indicator level of analysis need not be an indicator level of analysis relative to all multidimension signals present. That is, multidimension signals that are not or are not intended to be analyzed or acted upon in terms of a predetermined pattern as disclosed herein can be present in a level of analysis with other multidimension signals that are analyzed in terms of a predetermined pattern. The latter analysis renders that level of analysis an indicator level of analysis relative to the latter multidimension signals (that is, the “other” multidimension signals). Similarly, a given reporter signal level of analysis need not be a reporter signal level of analysis relative to all multidimension signals present. That is, multidimension signals that are not or are not intended to be analyzed or acted upon in terms of identifying reporter signals (or other multidimension signals) as disclosed herein can be present in a level of analysis with other multidimension signals (such as reporter signals) that are analyzed in terms of identifying reporter signals (or other multidimension signals). The latter analysis renders that level of analysis a reporter signal level of analysis relative to the latter multidimension signals (that is, the “other” multidimension signals). Further, a given level or round of analysis can be an indicator level of analysis relative to some multidimension signals present and a reporter signal level of analysis relative to other multidimension signals present.

In some forms of indicator level of analysis, reporter signals having a common property can be used with other multidimension signals that lack that common property. This difference can be the basis of the predetermined pattern used in the disclosed method. For example, a set of reporter signals where members of the set have a common property can be used together with one or more indicator signals that lack the common property. As another example, a set of reporter signals where members of the set have a common property can be used together with one or more other sets of reporter signals where the members of a given other set have a common property that differs from the common property of the first set of reporter signals. As another example, one or more sets of reporter signals where the members of each given set of reporter signals has a common property that differs from the common property of the members of the other sets of reporter signals can be used with one or more indicator signals that lack the common property of one or more or all of the sets of reporter signals.

In the disclosed multilevel analysis, an analysis level that involves identification of reporter signals (or other multidimension signals) can be referred to as a reporter signal level, reporter signal identification level, or reporter signal analysis. Some forms of multilevel analyses can be performed where one of the levels of analysis is a reporter signal level. In this way, the disclosed reporter signal levels of analysis can be combined with any other technique or method of processing or analysis of samples and analytes, either before or following the reporter signal analysis. Some forms of the disclosed method involve an indicator level followed by a reporter signal level. Multiple indicator levels and reporter signal levels can also be combined in the same assay or assay system.

Relationships of common properties can be illustrated using mass (or mass-to-charge ratio). As one example, a set of reporter signals where members of the set have the same mass (the common property) can be used together with one or more indicator signals that have a different mass from the reporter signals (and thus lack the common property). As another example, a set of reporter signals where members of the set have the same mass (the common property) can be used together with one or more other sets of reporter signals where the members of a given other set have a mass (common property) that differs from the mass (common property) of the first set of reporter signals. As another example, one or more sets of reporter signals where the members of each given set of reporter signals has a mass (common property) that differs from the mass (common property) of the members of the other sets of reporter signals can be used with one or more indicator signals that have a different mass than the members of one or more or all of the sets of reporter signals. The indicator signals thus lack the common property of the reporter signals. In these examples, all of the members of a given set of reporter signals can have the same mass (thus making mass the common property).

The same relationships can exist for mass-to-charge ratio, for example. It should be understood that mass differences result in mass-to-charge ratio differences and that such mass-to-charge ratio differences are proportional to mass differences when the charge on different species is the same. In this way, reference to mass, mass differences and relative mass should also be considered references to mass-to-charge ratios, mass-to-charge ratio differences, and relative mass-to-charge ratios. As one example, a set of reporter signals where members of the set have the same mass-to-charge ratio (the common property) can be used together with one or more indicator signals that have a different mass-to-charge ratio from the reporter signals (and thus lack the common property). As another example, a set of reporter signals where members of the set have the same mass-to-charge ratio (the common property) can be used together with one or more other sets of reporter signals where the members of a given other set have a mass-to-charge ratio (common property) that differs from the mass-to-charge ratio (common property) of the first set of reporter signals. As another example, one or more sets of reporter signals where the members of each given set of reporter signals has a mass-to-charge ratio (common property) that differs from the mass-to-charge ratio (common property) of the members of the other sets of reporter signals can be used with one or more indicator signals that have a different mass-to-charge ratio than the members of one or more or all of the sets of reporter signals. The indicator signals thus lack the common property of the reporter signals. In these examples, all of the members of a given set of reporter signals can have the same mass-to-charge ratio (thus making mass-to-charge ratio the common property).

The use of reporter signals in the disclosed methods allows efficient analysis of a large number of different samples and/or analytes in the same assay. For example, different reporter signals (belonging to a set of reporter signals where the reporter signals have a common property) can be used to label analytes in different samples or to label different analytes. Other multidimension signals can be used to label analytes in other samples or to label other analytes. For example, different indicator labels (each of which differs from the reporter signals in the common property) can be used to label analytes in the other samples. As another example, different reporter signals (belonging to a second set of reporter signals where the reporter signals have a common property that differs from the common property of the reporter signals of the first set) can be used to label analytes in the other samples. When the first level of analysis (the indicator level of analysis) is performed, the reporter signals will differ from the other multidimension signals in the common property and this known difference can be the basis of a predetermined pattern. In the next level of analysis (the reporter signal level of analysis), triggered by the predetermined pattern, the reporter signals in the portion of the analysis sample indicated by the predetermined pattern can be altered and the altered forms of the reporter signals detected and distinguished. Each set of reporter signals alone allows differential detection of many samples and/or analytes and use of multiple sets of reporter signals increases the level of multiplexing possible. In addition, each different set of reporter signals can contribute to the pattern generated in the indicator level of analysis, thus making the reporter signal level of analysis more efficient.

Some forms of the method can involve labeling analytes in a first sample or a first set of samples with a set of multidimension signals, labeling analytes in a second sample or second set of samples with a different set of multidimension signal, mixing the first and second samples to form an analysis sample, analyzing the multidimension signal-labeled analytes in the analysis sample to identify one or more predetermined patterns that result from the multidimension signal, where identification of the one or more predetermined patterns identifies one or more portions of the analysis sample, analyzing the multidimension signal in one or more of the one or more identified portions of the analysis sample to identify the multidimension signal present in identified portion of the analysis sample. In some forms of the method, one or both of the sets of multidimension signals can be a set of reporter signals and the analysis of the multidimension signals in one or more of the one or more identified portions of the analysis sample identifies the reporter signals. Either or both sets of multidimension signals can include both reporter signals and indicator signals, a set of reporter signals and an indicator signal, a reporter signal and a set of indicator signals or a set of reporter signals and a set of indicator signals.

Additional non-limiting forms of the disclosed method can involve one to three steps. A filtering, selection, or separation step to separate isobaric multidimension signals (and the attached analytes or proteins) from other molecules that may be present (e.g., based on mass-to-charge ratio), an optional fragmentation step to fragment the multidimension signals to produce fragments having different masses, and a detection step that detects a multidimension signal, labeled analyte or labeled protein, or both; or that distinguishes different multidimension signals, different labeled analytes or different labeled proteins, or both based, for example, on their mass-to-charge ratios. The first stage filtering, selection, or separation step can be used to produce predetermined patterns that indicate whether the second, fragmentation stage should be performed and/or which portion(s) of the analyzed material can or should be analyzed in the fragmentation stage. The labeled analytes or labeled proteins preferably are distinguished and/or separated from other molecules based on some common property shared by the attached multidimension signals but not present in most (or, preferably, all) other molecules present. The labeled analytes or labeled proteins can also be distinguished and/or separated from other molecules based on a common property of the labeled analyte or labeled protein as a whole, such as the mass-to-charge ratio of the labeled analyte or protein. The separated labeled analytes are then treated and/or detected in such a way that the different multidimension signals, different labeled analytes or different labeled proteins, or both, are distinguishable. The different fragments will include the fragment of the multidimension signal and the fragmented labeled analyte or protein (made up of the analyte or protein and the remaining part of the multidimension signal). Either or both may be detected and will be characteristic of the initial labeled analyte. The method is best carried out using a tandem mass spectrometer, as described below. In such an instrument the isobaric multidimension signals are first filtered, then multidimension signals are fragmented (preferably by collision), and the fragments are distinguished and detected.

The disclosed methods are useful for sensitive detection of one or multiple analytes. In general, the methods involve the use of special label components, referred to as multidimension signals, that can be associated with, incorporated into, or otherwise linked to the analytes, or that can be used merely in conjunction with analytes, with no significant association between the analytes and multidimension signals. In some embodiments of the methods, the multidimension signals (or derivatives of the multidimension signals) are detected, thus indicating the presence of the associated analytes. In other embodiments, the analyte (or derivatives of the analytes) are detected along with the multidimension signals (or derivatives of the multidimension signals).

In some embodiments of the methods, sets of multidimension signals (e.g., reporter signals) can be used where two or more of the multidimension signals in a set have one or more common properties that allow the multidimension signals having the common property to be distinguished and/or separated from other molecules lacking the common property. In other embodiments, sets of multidimension signal/analyte conjugates (e.g., sets of reporter signal/analyte conjugates) can be used where two or more of the multidimension signal/analyte conjugates in a set have one or more common properties that allow the multidimension signal/analyte conjugates having the common property to be distinguished and/or separated from other molecules lacking the common property. In still other embodiments, analytes can be fragmented (prior to or following conjugation) to produce multidimension signal/analyte fragment conjugates (which can be referred to as fragment conjugates). In such cases, sets of fragment conjugates can be used where two or more of the fragment conjugates in a set have one or more common properties that allow the fragment conjugates having the common property to be distinguished and/or separated from other molecules lacking the common property. It should be understood that fragmented analytes can be considered analytes in their own right. In this light, reference to fragmented analytes is made for convenience and clarity in describing certain embodiments and to allow reference to both the base analyte and the fragmented analyte.

Multidimension signals (e.g., reporter signals or indicator signals) can also be in conjunction with analytes (such as in mixtures of multidimension signals and analytes), where no significant physical association between the multidimension signals and analytes occurs; or alone, where no analyte is present. In such cases, where multidimension signals are not or are no longer associated with analytes, sets of multidimension signals can be used where two or more of the multidimension signals in a set have one or more common properties that allow the multidimension signals having the common property to be distinguished and/or separated from other molecules lacking the common property.

In preferred embodiments, the disclosed methods involve two basic steps: a filtering, selection, or separation step to separate multidimension signals, labeled analytes, labeled proteins, or multidimension signal fusions from other molecules that may be present, and a detection step that detects or distinguishes different multidimension signals, different labeled analytes, different labeled proteins, different multidimension signal fusions, or all of these. The multidimension signals preferably are distinguished and/or separated from other molecules based on some common property shared by the multidimension signals but not present in most (or, preferably, all) other molecules present. The separated multidimension signals are then treated and/or detected such that the different multidimension signals are distinguishable. The first, filtering step (which can constitute an indicator level of analysis) can be used to produce predetermined patterns that indicate whether the second, detection step (which constitutes a reporter signal level of detection) should be performed and/or which portion(s) of the analyzed material can or should be analyzed in the detection step. Useful forms of the disclosed method involve association of multidimension signals with analytes of interest. Detection of the multidimension signals results in detection of the corresponding analytes. Thus, the disclosed method is a general technique for labeling and detection of analytes.

FIG. 1 is a diagram of an example of the use of multidimension signals. Two samples are labeled independently with two sets of multidimension signals (Label Set 1 and Label Set 2). The labeled samples are mixed, subjected to trypsin digestion (this will cleave proteins in the samples). The mixed, trypsinized sample is cleaned up with HPLC and then subjected to two rounds of mass spectrometry. Example 1 provides an example of an assay following the steps shown in FIG. 1.

A useful example of the logical flow of an example of the disclosed methods is shown in FIG. 4A. A mass spectrometry spectrum is collected (first box), and the spectrum is analyzed to detect a non-isobaric patterns (second box). The first two boxes correspond to an indicator level of analysis. The spectrum can be scanned for predetermined patterns (first circle). If a predetermined pattern is not detected, the indicator level of analysis is repeated for another sample or portion of sample (loop from first circle to first box). If a predetermined pattern is detected, a portion of the sample where the pattern was detected is sent for another level of analysis (first circle; downward arrow). A tandem mass spectrometry spectrum is collected on the portion of the sample (third box), and the spectrum is analyzed for information about the sample. The third and fourth boxes correspond to a reporter signal level of analysis. The entire analysis can be repeated on additional samples (loop from second circle to first box).

Using Example 1 as an example, the spectrum could be scanned for double peaks separated by 18 Daltons or multiples of 18 Daltons. Computer implemented methods for detection of this type of pattern are known (for example, pro-iCAT software, Applied Biosystems (product number WC026995; https://www.appliedbiosystems.com/catalog/myab/StoreCatalog/products/CategoryDetails.jsp?hierarchyID=101&category1st=111395&category2nd=111635&category3rd=112058)). If a predetermined pattern is not detected, the indicator level of analysis is repeated for another sample or portion of sample (circle; arrow on the left). If a predetermined pattern is detected, a portion of the sample where the pattern was detected is sent for another level of analysis (circle; downward arrow). A tandem mass spectrometry spectrum is collected on the portion of the sample (third box), and the spectrum is analyzed for information about the sample. The last two boxes correspond to a reporter signal level of analysis. FIG. 4 is an example of the analysis that can be involved in and between the two mass spectrometry stages shown in FIG. 1. Example 1 provides an example use of the logical flow shown in FIG. 4.

For simplicity, a single protein has been shown in FIG. 4. This logical flow can be extended to larger collections of proteins. Further, this two sample experiment can be extended simply by parallelizing the sample preparation and pooling strategy. As an example, consider a population of “normal” input samples compared to a population of “treated” samples shown in FIG. 5. FIG. 5 is an example of the method shown in FIG. 1 where two different sample sets (Control samples and Tester samples) are labeled with different members of two different sets of multidimension signals (Label Set 1 and Label Set 2). In this example, 5 different Tester samples are each labeled with a different member of Label Set 2 and 7 different Control samples are each labeled with a different member of Label Set 1. The label sets can be, for example, the label sets shown in Table 3. The correlation between the label sets and the Control and Tester samples is for clarity and does not represent a limitation of the method. The labeled samples are mixed, subjected to trypsin digestion (this will cleave proteins in the samples). The mixed, trypsinized sample is cleaned up with HPLC and then subjected to two rounds of mass spectrometry. A preferred form of the method mixes labeled Control and Tester samples across Label Set 1 and Label Set 2.

In this design, observation of the non-isobaric label pattern will trigger measurements from a population of input samples. Population based statistical inference about the control and tester states can be built into the assay. For example, higher statistical confidence can come from more measurements. A preferred form would mix Control and Tester samples across the sets of multidimension signals (Label Sets) to counter the cases where a particular protein might be absent in a Control or Tester sample. That is, each set of mutidimension signals can include one or more Control and one or more Tester samples. This can reduce, eliminate or control for bias among the sets.

There is no particular limitation to the number of non-isobaric elements in the disclosed methods. In this example, two sets of multidimension signals that are not isobaric to each other were used, thus providing two non-isobaric elements to the assay. However, as the number of non-isobaric elements increases, the MS spectrum becomes more complex. It is preferred that the separation of ions to be distinguished is greater than the resolving power of the mass spectrometer used to make the measurements.

It is not necessary that the non-isobaric and isobaric elements of the system be embodied in the same multidimension signals or sets of multidimension signals. In the above example the non-isobaric nature was imparted through the inclusion or exclusion of heavy glycine in molecules of otherwise the same composition. As an example, this method may use as single set of isobaric multidimension signals (e.g. Label Set 1) and a second multidimension signal which imparts the non-isobaric nature of the method. For example, acetylation of primary amines is known (Wetzel et al., Bioconjugate Chem 1: 114-122 (1990)). The heavy versus light non-isobaric character can be introduced through reaction with acetic anhydride. As an example, a three element non-isobaric system can be created by labeling with acetic anhydride (light), with the perdeuterated analog of acetic anhydride (heavy), or with the perfluoronated analog of acetic anhydride (really heavy). In this case the MS spectra could be scanned for the pattern corresponding to this set of three non-isobaric labels, then the MS/MS spectra would resolved the differences in the members of these non-isobaric sets.

The non-isobaric nature of the method can be incorporated by metabolic means. For example, cells grown on heavy or light lysine culture will incorporate these heavy or light residues respectively. There is no limitation to the inclusion of more than two labels in this method.

As mentioned above, a preferred form of the disclosed method involves filtering of isobaric multidimension signals (and the attached analytes or proteins) from other molecules based on mass-to-charge ratio, fragmentation of the multidimension signals to produce fragments having different masses, and detection of the different fragments based on their mass-to-charge ratios. The first stage filtering can be used to produce predetermined patterns that indicate whether the second, fragmentation stage should be performed and/or which portion(s) of the analyzed material can or should be analyzed in the fragmentation stage.

The method is best carried out using a tandem mass spectrometer, as described above. The same sample can be analyzed both with and without fragmentation (by operating with and without collision gas), and the results compared to detect shifts in mass-to-charge ratio. Both the unfragmented and fragmented results should give diagnostic peaks, with the combination of peaks both with and without fragmentation confirming the multidimension signal (and analyte) involved. Such distinctions are accomplished by using appropriate sets of isobaric multidimension signals and allow large scale multiplexing in the detection of analytes.

The disclosed method is particularly well suited to the use of a MALDI-QqTOF mass spectrometer. The method enables highly multiplexed analyte detection, and very high sensitivity. Preferred tandem mass spectrometers are described by Loboda et al., Design and Performance of a MALDI-QqTOF Mass Spectrometer, in 47th ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000). In such an instrument the sample is ionized in the source (MALDI, for example) to produce charged ions; it is preferred that the ionization conditions are such that primarily a singly charged parent ion is produced. First and third quadrupoles, Q0 and Q2, will be operated in RF only mode and will act as ion guides for all charged particles, second quadrupole Q1 will be operated in RF+DC mode to pass only a particular mass-to-charge (or, in practice, a narrow mass-to-charge range). This quadrupole selects the mass-to-charge ratio, (m/z), of interest. The collision cell surrounding Q2 can be filled to appropriate pressure with a gas to fracture the input ions by collisionally induced dissociation (normally the collision gas is chemically inert, but reactive gases are contemplated). Preferred molecular systems utilize multidimension signals that contain scissile bonds, labile bonds, or combinations, and these bonds will be preferentially fractured in the Q2 collision cell.

A MALDI source is preferred for the disclosed method because it facilitates the multiplexed analysis of samples from heterogeneous environments such as arrays, beads, microfabricated devices, tissue samples, and the like. An example of such an instrument is described by Qin et al., A practical ion trap mass spectrometerfor the analysis ofpeptides by matrix-assisted laser desorption/ionization., Anal. Chem., 68:1784-1791 (1996). For homogeneous assays electrospray ionization (ESI) sources will work very well. Electrospray ionization source instruments interfaced to LC systems are commercially available (for example, QSTAR from PE-SCIEX, Q-TOF from Micromass). It is of note that the ESI sources are operated such that they tend to produce multiply charged ions, doubly charged ions would be most common for ions in the disclosed method. Such doubly charged ions are well known in the art and present no limitation to the disclosed method. TOF analyzers and quadrupole analyzers are preferred detectors over sector analyzers. Tandem in time ion trap systems such as Fourier Transform Ion Cyclotron Resonance (FT-ICR) mass spectrometers also may be used with the disclosed method.

A number of elements contribute to the sensitivity of the disclosed method. The filter quadrupole, Q1, selects a narrow mass-to-charge ratio and discriminates against other mass-to-charge ions, significantly decreasing background from non germane ions. For example, for a sample containing a distribution of mass-to-charges of width 3000 Da, a mass-to-charge transmission window of 2 Da applied to this distribution can improve the signal to noise by at least a factor of 3000/2=1500. Once the parent ion is selected by quadrupole Q1, fragmentation of the parent ion, preferably into a single charged daughter ion, has the advantage over systems which fragment the parent into a number of daughter ions. For example, a parent fragmented into 20 daughter ions will yield signals that are on average 1/20^{th} the intensity of the parent ions. For a parent to single daughter system there will not be this signal dilution.

This preferred system for use with the disclosed method has a high duty cycle, and as such good statistics can be collected quickly. For the case where a single set of isobaric parents is used, the multiplexed detection is accomplished without having to scan the filter quadrupole (although such a scan is useful for single pass analysis of a complex protein sample with multiple labeled proteins). Electrospray sources can operate continuously, MALDI sources can operate at several kHz, quadrupoles operate continuously, and time of flight analyzers can capture the entire mass-to-charge region of interest at several kHz repetition rate. Thus, the overall system can acquire thousands of measurements per second. For throughput advantage in a multiplexed assay the time of flight analyzer has an advantage over a quadruple analyzer for the final stage because the time of flight analyzer detects all fragment ions in the same acquisition rather than requiring scanning (or stepping) over the ions with a quadrupole analyzer.

Instrumental improvements including addition of laser ports along the flight path to allow intersection of the proteins with additional laser(s) open additional fragmentation avenues through photochemical and photophysical processes (for example, selective bond cleavage, selective ionization). Use of lasers to fragment the proteins after the filter stage will enable the use of the very high throughput TOF-TOF instruments (50 kHz to 100 kHz systems).

The disclosed method is compatible with techniques involving cleavage, treatment, or fragmentation of a bulk sample in order to simplify the sample prior to introduction into the first stage of a multistage detection system. The disclosed method is also compatible with any desired sample, including raw extracts and fractionated samples.

### FORMS AND EMBODIMENTS OF THE DISCLOSED MATERIALS

A. Multidimension Molecule Labeling

In one form of the disclosed method, referred to as multidimension molecule labeling (MDML), multidimension signals are first associated with analytes to be detected and/or quantitated, and then dissociated and detected. The dissociated multidimension signals are subjected to an indicator level of analysis and a reporter signal level of analysis. As an example, a multidimension signal can be associated with a specific binding molecule that interacts with the analyte of interest. Such a combination is referred to as a multidimension molecule. The specific binding molecule in the multidimension molecule interacts directly with the analyte thus associating the multidimension signal with the analyte. Alternatively, a multidimension signal can be associated with an analyte indirectly. Regardless of whether the interaction of the multidimension signal with the specific binding molecule is direct or indirect, the interaction of the specific binding molecules with the analytes allows the multidimension signals to be associated with the analytes. The method of the invention can be performed such that the fact of association between the analyte and multidimension signal is part of the information obtained when the multidimension signal is detected. In other words, the fact that the multidimension signal may be dissociated from the analyte for detection does not obscure the information that the detected multidimension signal was associated with the analyte. Multidimension signals used and/or detected using different techniques (such as multidimension signal labeling, reporter signal calibration, and multidimension signal fusions) can be used in and/or combined with MDML.

The disclosed method increases the sensitivity and accuracy of detection of an analyte or protein of interest. Preferred forms of the disclosed method make use of multistage detection systems to increase the resolution of the detection of molecules having very similar properties. In one example, the method involves at least two stages. The first stage is filtration or selection that allows passage or selection of multidimension signals, labeled analytes or proteins, or multidimension signal fusions (that is, a subset of the molecules present), based upon intrinsic properties of the multidimension signals (and the attached analytes or proteins), and discrimination against all other molecules. The subsequent stage(s) further separate(s) and/or detect(s) the multidimension signals, labeled analytes or proteins, or multidimension signal fusions which were filtered in the first stage. A key facet of this method is that a multiplexed set of multidimension signals, labeled analytes or proteins, or multidimension signal fusions will be selected by the filter and the attached multidimension signals will be subsequently cleaved, decomposed, reacted, or otherwise modified to realize the identities and/or quantities of the fragmented multidimension signals, the fragmented labeled analytes or proteins, and/or fragmented multidimension signal fusions in further stages. There is a correspondence between the multidimension signal and the detected daughter fragment.

B. Multidimension Signal Labeling

In another form of the disclosed method, referred to as multidimension signal labeling (MDSL) or multidimension signal protein labeling, multidimension signals are used for sensitive detection of one or multiple analytes or proteins. The method involves detection of analytes or proteins by detecting a multidimension signal, labeled analyte or labeled protein, or both; or by distinguishing different multidimension signals, different labeled analytes or different labeled proteins, or both. In the method, analytes or proteins labeled with multidimension signals are analyzed using the multidimension signals to distinguish the labeled analytes or proteins (where the analytes or proteins are labeled with the multidimension signals). The multidimension signals are subjected to an indicator level of analysis and/or a reporter signal level of analysis. Detection of the multidimension signals results in detection of the corresponding labeled analytes (where the analytes are labeled with the multidimension signals) or corresponding labeled proteins (where the proteins are labeled with the multidimension signals). Detection of the labeled analytes or labeled proteins results in detection of the corresponding analytes and proteins. The detected analyte(s) can then be analyzed using known techniques. The use of the multidimension signals as labels thus provide a unique analyte/label composition or unique protein/label composition that can specifically identify the analyte(s) or protein(s). Thus, multidimension signal labeling and multidimension signal protein labeling are general techniques for labeling, detection, and quantitation of analytes and proteins.

Note that although reference is made above and elsewhere herein to detection of a “protein” or “proteins,” the disclosed method and compositions encompass proteins, peptides, and fragments of proteins or peptides. Thus, reference to a protein herein is intended to refer to proteins, peptides, and fragments of proteins or peptides unless the context clearly indicates otherwise.

In some embodiments, the multidimension signals are designed to be fragmented to yield fragments of similar charge but different mass. This allows each labeled analyte or protein (and/or each multidimension signal or multidimensional signal fusion (e.g., a reporter signal fusion)) in a set to be distinguished by the different mass-to-charge ratios of the fragments of the multidimension signals. This is possible since, although the unfragmented multidimension signals in a set are isobaric, the fragments of the different multidimension signals are not. In the disclosed method, this allows each protein/multidimension signal combination (or analyte/multidimensional signal combination or multidimension signal fusion) to be distinguished by the mass-to-charge ratios of the protein/multidimension signals after fragmentation of the multidimension signal.

Thus, the labeled analyte(s) or labeled protein(s) can be fragmented prior to analysis. An analyte or protein sample to be analyzed can also be subjected to fractionation or separation to reduce the complexity of the samples. Fragmentation and fractionation can also be used together in the same assay. Such fragmentation and fractionation can simplify and extend the analysis of the analytes.

Multidimension signals can be coupled or directly associated with an analyte or protein. For example, a multidimension signal can be coupled to an analyte or protein via reactive groups, or a multidimension molecule (composed of a specific binding molecule and a multidimension signal) can be associated with an analyte or protein. The multidimension signals can be attached to analytes or to proteins in any manner. For example, multidimension signals can be covalently coupled to proteins through a sulfur-sulfur bond between a cysteine on the protein and a cysteine on the multidimension signal. Many other chemistries and techniques for coupling compounds to analytes are known and can be used to couple multidimension signals to analytes. For example, coupling can be made using thiols, epoxides, nitriles for thiols, NHS esters, isothiocyanates for amines, and alcohols for carboxylic acids. Multidimension signals can be attached to analytes either directly or indirectly, for example, via a linker.

Multidimension signals, or constructs containing multidimension signals, also can be attached or coupled to analytes by ligation. Methods for ligation of nucleic acids are well known (see, for example, Sambrook et al. Molecular Cloning: A Laboratory Manual, second edition, 1989, Cold Spring Harbor Laboratory Press, New York.), and efficient protein ligation is known (see, for example, Dawson et al., “Synthesis of proteins by native chemical ligation” Science 266, 776-9 (1994); Hackeng et al., “Chemical synthesis and spontaneous folding of a multidomain protein: anticoagulant microprotein S” Proc Natl Acad Sci USA 97:14074-8 (2000); Dawson et al., “Synthesis of Native Proteins by Chemical Ligation” Ann. Rev. Biochem. 69:923-960 (2000); U.S. Pat. No. 6,184,344; PCT Publication WO 98/28434).

Alternatively, a multidimension signal can be associated with an analyte indirectly. In this mode, a “coding” molecule containing a specific binding molecule and a coding tag can be associated with the analyte (via the specific binding molecule). Alternatively, a coding tag can be coupled or directly associated with the analyte. Then a multidimension signal associated with a decoding tag (such a combination is another form of multidimension molecule) is associated with the coding molecule through an interaction between the coding tag and the decoding tag. An example of this interaction is hybridization where the coding and decoding tags are complementary nucleic acid sequences. The result is an indirect association of the multidimension signal with the analyte. This mode has the advantage that all of the interactions of the multidimension signals with the coding molecule can be made chemically and physically similar by using the same types of coding tags and decoding tags for all of the coding molecules and multidimension molecules in a set.

Multidimension signals used in MDSL can generate one or more predetermined patterns in indicator levels of analysis. Where the multidimension signals are coupled to analytes, the pattern can be generated by the combination of multidimension signals and analytes.

Multidimension signals, such as reporter signals, can be fragmented, decomposed, reacted, derivatized, or otherwise modified, preferably in a characteristic way. This allows an analyte or protein to which the multidimension signal is attached or fused to be identified by the correlated detection of the labeled analyte or labeled protein and one or more of the products of the labeled analyte or protein following fragmentation, decomposition, reaction, derivatization, or other modification of the multidimension signal (the labeled analyte is the analyte/multidimension signal combination while the labeld protein is the protein/multidimension signal combination). The protein can also be identified by the correlated detection of the multidimension signal fusion and one or more of the products of the multidimension signal fusion following fragmentation, decomposition, reaction, derivatization, or other modification of the multidimension signal peptide. The alteration of the multidimension signal will alter the labeled analyte or the labeled protein in a characteristic and detectable way. Together, the detection of a characteristic labeled analyte or labeled protein and a characteristic product of the labeled analyte or labeled protein can uniquely identify the analyte or protein. In this way, using the disclosed method and materials, one or more analytes or proteins can be detected, either alone or together (for example, in a multiplex assay). Further, one or more analytes or proteins in one or more samples can be detected in a multiplex manner. For example, for mass spectrometry multidimension signals, the multidimension signals are fragmented to yield fragments of similar charge but different mass.

In some embodiments, multidimension signals, such as reporter signals, are used in sets where all the multidimension signals in the set have similar properties (such as similar mass-to-charge ratios). The similar properties allow the multidimension signals to be distinguished and/or separated from other molecules lacking one or more of the properties. In some embodiments, the multidimension signals in a set have the same mass-to-charge ratio (m/z). That is, the multidimension signals in a set are isobaric. This allows the multidimension signals (or any analytes to which they are attached) to be separated precisely from other molecules based on mass-to-charge ratio. The result of the filtering is a huge increase in the signal to noise ratio (S/N) for the system, allowing more sensitive and accurate detection. Alternatively, or in addition, multidimension signals can be used in sets such that the resulting labeled analytes will have similar properties allowing the labeled analytes to be distinguished and/or separated from other molecules lacking one or more of the properties.

Analytes can be detected using the disclosed multidimension signals in a variety of ways. For example, the analyte and attached multidimension signal can be detected together, one or more fragments of the analyte and the attached multidimension signal(s) can be detected together, the fragments of the multidimension signal can be detected, or a combination.

One non-limiting form of the disclosed method involves correlated detection of the multidimension signals both before and after fragmentation of the multidimension signal. This allows labeled analytes or proteins to be detected and identified via the change in labeled analyte or protein. That is, the nature of the multidimension signal detected (non-fragmented versus fragmented) identifies the analyte or proteins as labeled. Where the analytes or proteins and multidimension signals are detected by mass-to-charge ratio, the change in mass-to-charge ratio between fragmented and non-fragmented samples provides the basis for comparison. Such mass-to-charge ratio detection is preferably accomplished with mass spectrometry.

As an example, an analyte in a sample can be labeled with multidimension signal designed as a mass spectrometry label. The labeled analyte can be subjected to mass spectrometry. A peak corresponding to the analyte/multidimension signal will be detected. Analytes labeled with different multidimension signals in the assay can generate related peaks that form a pattern. Such a pattern can be used to indicate whether a further level of analysis can or should be performed and/or which portion(s) of the analyzed material can or should be analyzed in a further level of analysis. Fragmentation of the multidimension signal in the mass spectrometer (preferably in a collision cell) results in a shift in the peak corresponding to the loss of a portion of the attached multidimension signal, the appearance of a peak corresponding to the lost fragment, or a combination of both events. Significantly, the shift observed will depend on which multidimension signal is on the analyte since different multidimension signals will, by design, produce fragments with different mass-to-charge ratios. The combination event of detection of the parent mass-to-charge (with no collision gas) and the mass-to-charge corresponding to the loss of the fragment from the multidimension signal (with collision gas) indicates a labeled analyte. The identity of the analyte can be determined by standard mass spectrometry techniques, such as compositional analysis.

A powerful form of the disclosed method is use of analytes or proteins labeled with multidimension signals or use of multidimension signal fusions to assay multiple samples (for example, time series assays or other comparative analyses). Knowledge of the temporal response of a biological system following perturbation is a very powerful process in the pursuit of understanding the system. To follow the temporal response, a sample of the system is obtained (for example, cells from a cell culture, mice initially synchronized and sacrificed) at determined times following the perturbation. Knowledge of spatial analyte profiles (for example, relative position within a tissue section) is a very powerful process in the pursuit of understanding the biological system.

In the disclosed method a series of samples can each be labeled with a different multidimension signal from a set of multidimension signals. Non-limiting multidimension signals for this purpose would be those using differentially distributed mass. In particular, the use of stable isotopes may be used to ensure that members of the set of multidimension signals would behave chemically identically and yet would be distinguishable.

The labeled analytes may be detected using mass spectrometry which allows sensitive distinctions between molecules based on their mass-to-charge ratios. The disclosed multidimension signals can be used as general labels in myriad labeling and/or detection techniques. One or more sets of isobaric multidimension signals can be used for multiplex labeling and/or detection of many analytes since the multidimension signal fragments can be designed to have a large range of masses, with each mass individually distinguishable upon detection. Further, use of more than one isobaric multidimension signal set where the sets are not isobaric to each other allows both generation of predetermined patterns and a powerful means to increase the multiplexing potential of the disclosed methods. Where the same analyte or type of analyte is labeled with a set of isobaric multidimension signals (by, for example, labeling the same analyte in different samples), the set of labeled analytes that results from use of an isobaric set of multidimension signals will also be isobaric. Analogously, non-isobaric multidimension signals and sets of multidimension signals that are not isobaric to the other sets can be used to label the same analyte (by, for example, labeling the same analyte in different samples). The result will be labeled analytes that are not isobaric; a pattern of labeled analytes having different masses will be generated. Use of combinations of isobaric and non-isobaric multidimension signals or sets of multidimension signals to label the same analyte in different samples can generate a pattern of masses in an indicator level of analysis. Fragmentation of the multidimension signals in a reporter signal level of analysis will split the set of labeled analytes into individually detectable labeled proteins of characteristically different mass.

The disclosed method can be used in many modes. For example, the disclosed method can be used to detect a specific analyte or protein (in a specific sample or in multiple samples) or multiple analytes or proteins (in a single sample or multiple samples). In each case, the analyte(s) or protein(s) to be detected can be separated either from other, unlabeled analytes or from other molecules lacking a property of the labeled analyte(s) to be detected. For example, analytes or proteins in a sample can be generally labeled with multidimension signals and some analytes or proteins can be separated on the basis of some property of the analytes or proteins. For example, the separated analytes or proteins could have a certain mass-to-charge ratio (separation based on mass-to-charge ratio will select both labeled and unlabeled analytes having the selected mass-to-charge ratio). As another example, all of the labeled analytes or labeled proteins can be distinguished and/or separated from unlabeled molecules based on a feature of the multidimension signal such as an affinity tag. Where different affinity tags are used, some labeled analytes can be distinguished and/or separated from others. Multidimension signal labeling allows profiling of analytes and cataloging of analytes.

In one mode of the disclosed method, multiple analytes or proteins in multiple samples are labeled where all of the analytes or proteins in a given sample are labeled with the same multidimension signal. That is, the multidimension signal is used as a general label of the analytes or proteins in a sample. Each sample, however, uses a different multidimension signal. This allows samples as a whole to be compared with each other. By additionally separating or distinguishing different analytes or proteins in the samples, one can easily analyze many analytes or proteins in many samples in a single assay. For example, proteins in multiple samples can be labeled with multidimension signals as described above, and the samples mixed together. If some or all of the various labeled proteins are separated by, for example, association of the proteins with antibodies on an array, the presence and amount of a given protein in each of the samples can be determined by identifying the multidimension signals present at each array element. If the protein corresponding to a given array element was present in a particular sample, then some of the protein associated with that array element will be labeled with the multidimension signal used to label that particular sample. Detection of that multidimension signal will indicate this. This same relationship holds true for all of the other samples. Further, the amount of multidimension signal detected can indicate the amount of a given protein in a given sample, and the simultaneous quantitation of protein in multiple samples can provide a particularly accurate comparison of the levels of the proteins in the various samples.

Optionally, the selection step can be preceded by fractionation step where a subset of analytes, including the analytes that are, or will be, labeled, are separated from other components in a sample. For example, proteins having an SH2 domain can be separated from other proteins in a cell sample prior to the selection step. Such a step, although not necessary, can improve the selection step by reducing the number of extraneous molecules present.

In preferred embodiments, multidimension signals (or reporter signals or indicator signals) are used in sets where all the multidimension signals (or reporter signals or indicator signals) in the set have similar properties (such as similar mass-to-charge ratios). The similar properties allow the multidimension signals (or reporter signals or indicator signals) to be distinguished and/or separated from other molecules lacking one or more of the properties. In some embodiments, the multidimension signals (or reporter signals or indicator signals) in a set have the same mass-to-charge ratio (m/z). That is, the multidimension signals (or reporter signals or indicator signals) in a set are isobaric. This allows the multidimension signals (or reporter signals or indicator signals, or any proteins to which they are attached) to be separated precisely from other molecules based on mass-to-charge ratio. The result of the filtering is a huge increase in the signal to noise ratio (S/N) for the system, allowing more sensitive and accurate detection. Alternatively, or in addition, multidimension signals (or reporter signals or indicator signals) can be used in sets such that the resulting labeled proteins will have similar properties allowing the labeled proteins to be distinguished and/or separated from other molecules lacking one or more of the properties.

Proteins can be detected using the disclosed multidimension signals in a variety of ways. For example, the protein and attached multidimension signal can be detected together, one or more peptides of the protein and the attached multidimension signal(s) can be detected together, the fragments of the multidimension signal can be detected, or a combination. Preferred detection involves detection of the protein/multidimension signal or peptide/multidimension signal both before and after fragmentation of the multidimension signal.

As an example, a protein in a sample can be labeled with multidimension signal designed as a mass spectrometry label. The labeled protein can be subjected to tryptic digest followed by mass spectrometry of the resulting materials. A peak corresponding to the tryptic fragment containing the multidimension signal will be detected. Fragmentation of the multidimension signal in the mass spectrometer (preferably in a collision cell) would result in a shift in the peak corresponding to the loss of a portion of the attached multidimension signal, the appearance of a peak corresponding to the lost fragment, or a combination of both events. Significantly, the shift observed will depend on which multidimension signal is on the protein since different multidimension signals will, by design, produce fragments with different mass-to-charge ratios. The combination event of detection of the parent mass-to-charge (with no collision gas) and the mass-to-charge corresponding to the loss of the fragment from the multidimension signal (with collision gas) indicates a labeled protein. The combination event may be carried out in an analogous fashion to the detection of phosphorylation sites described above. The identity of the tryptic fragment of the protein can be determined by standard mass spectrometry techniques, such as compositional analysis and peptide sequencing.

Not all labeled analyte fragments or labeled protein fragments that can be made in the disclosed method from a protein sample will be unique. Because some proteins have common motifs that may be identical in different proteins, some protein fragments or peptides produced from a sample will be identical although they were derived from different proteins. For example, some families of related proteins have such common motifs or common amino acid sequences. Thus, in some embodiments of the disclosed method, detection of a characteristic labeled protein may be the result of detection of a common portion of related proteins. Such a result can be an advantage when detection of the family of proteins is desired. Alternatively, such collective detection of related proteins can be avoided by focusing on detection of unique fragments (that is, non-identical portions) of the proteins in the family. For convenience, as used herein, detection of a common portion of multiple related proteins is intended to be encompassed by reference to detection of a unique protein, labeled protein, or other component, unless the context clearly indicates otherwise.

In the disclosed method a series of samples can each be labeled with a different multidimension signal from a set of multidimension signals. Preferred multidimension signals for this purpose would be those using differentially distributed mass. In particular, the use of stable isotopes is preferred to ensure that members of the set of multidimension signals would behave chemically identically and yet would be distinguishable. An exemplary set of labels could be as shown in Table 1, where each of five time points could be labeled with one of the five indicated labels and the mixture of the samples could be read out simultaneously. The unfragmented labels are SEQ ID NO: 1 and the fragmented labels are amino acids 7-12 of SEQ ID NO:1.

TABLE 1

| | | | Fragment |
|---|---|---|---|
| | Mass | Fragment | mass |
| Sequence | (amu) | Sequence | (amu) |
| CG*G*G*G*DPGGGGR | 949 | PGGGGR | 499 |
| CG*G*G*GDPGGGG*R | 949 | PGGGG*R | 500 |
| CG*G*GGDPGGG*G*R | 949 | PGGG*G*R | 501 |
| CG*GGGDPGG*G*G*R | 949 | PGG*G*G*R | 502 |
| CGGGGDPG*G*G*G*R | 949 | PG*G*G*G*R | 503 |

In the disclosed method, these labels would be used in combination with one or more other multidimension labels that, together with the isobaric labels, would form predetermined patterns. The labeled proteins are preferably detected using mass spectrometry which allows sensitive distinctions between molecules based on their mass-to-charge ratios. The disclosed multidimension signals can be used as general labels in myriad labeling and/or detection techniques. A set of isobaric multidimension signals can be used for multiplex labeling and/or detection of many proteins since the multidimension signal fragments can be designed to have a large range of masses (or mass-to-charge ratios), with each mass (or mass-to-charge ratio) individually distinguishable upon detection. Where the same analyte, type of analyte, same protein, or type of protein is labeled with a set of isobaric multidimension signals (by, for example, labeling the same protein in different samples), the set of labeled analytes or labeled proteins that results from use of an isobaric set of multidimension signals will also be isobaric. Fragmentation of the multidimension signals will split the set of labeled analytes or labeled proteins into individually detectable labeled analytes or proteins of characteristically different mass.

The method allows detection of analytes, proteins, peptides and protein fragments where detection provides some information on the sequence or other structure of the analytes, protein or peptide detected. For example, the mass or mass-to-charge ratio, the amino acid composition, or amino acid sequence of the protein can be determined. The set of analytes, proteins, peptides and/or protein fragments detected in a sample using particular multidimension signals will produce characteristic sets of analyte, protein and peptide information. The method allows a complex sample of analytes or proteins to be cataloged quickly and easily in a reproducible manner. The disclosed method also should produce two “signals” for each analyte, protein, peptide, or peptide fragment in the sample: the original labeled analyte or labeled protein and the altered form of the labeled analyte or protein. This can allow comparisons and validation of a set of detected analytes, proteins and peptides.

A preferred form of the disclosed method involves detection of labeled analytes or proteins in two or more samples or proteins in the same assay. This allows simple and consistent detection of differences between the analytes or proteins in the samples. Differential detection is accomplished by labeling the analytes or proteins in each sample with a different multidimension signal. Preferably, the different multidimension signals used for the different samples will make up an isobaric set. In this way, the same labeled analyte or labeled protein in each sample will have the same mass-to-charge ratio as that labeled analyte or labeled protein in a different sample. Upon fragmentation of the multidimension signals, however, each of the fragmented labeled analytes or proteins in the different samples will have a different mass-to-charge ratio and thus each can be separately detected. All can be detected in the same measurement. This is a tremendous advantage in both time and quality of the data. For example, since the samples are assayed in a single run, there is no need to correct or normalize the results of different samples assayed in different runs. This allows accurate comparisons of the relative amounts of the same analyte in different samples since that are measured in the same run. There would be no differences to cause inconsistency between the samples.

A preferred use for this multiple sample mode of the disclosed method is the analysis of a time series of samples. Such series are useful for detecting changes in a sample or reaction over time. For example, changes in analyte or protein levels in a cell culture over time after addition of a test compound can be assessed. In this mode, different time point samples are labeled with different multidimension signals, preferably making up an isobaric set. In this way, the same labeled analyte or protein for each time point will have the same mass-to-charge ratio as that labeled analyte or protein from a different time point. Upon fragmentation of the multidimension signals, however, each of the fragmented labeled analytes or proteins from the different time points will have a different mass-to-charge ratio and thus each can be separately detected.

The disclosed method can also be used to gather and catalog information about unknown analytes and proteins. This analyte or protein discovery mode can easily link the presence or pattern of analytes or proteins with their analysis. For example, a sample of labeled analytes or proteins can be compared to analytes in one or more other samples. Analytes or proteins that appear in one or some samples but not others can be analyzed using conventional techniques. The object analytes or proteins will be distinguishable from others by virtue of the disclosed labeling, detection, and quantitation. This mode of the method is preferably carried out using mass spectrometry.

In some embodiments, the disclosed method allows a complex sample of analytes or proteins to be quickly and easily cataloged in a reproducible manner. Such a catalog can be compared with other, similarly prepared catalogs of other analyte or proteins samples to allow convenient detection of differences between the samples. The catalogs, which incorporate a significant amount of information about the analyte or proteins samples, can serve as fingerprints of the samples which can be used both for detection of related analyte or protein samples and comparison of analyte or protein samples. For example, the presence or identity of specific organisms can be detected by producing a catalog of analytes and/or proteins of the test organism and comparing the resulting catalog with reference catalogs prepared from known organisms. Changes and differences in analyte and/or proteins patterns can also be detected by preparing catalogs of analytes or proteins from different cell samples and comparing the catalogs. Comparison of analyte and/or proteins catalogs produced with the disclosed method is facilitated by the fine resolution that can be provided with, for example, mass spectrometry.

Each labeled analyte or protein processed in the disclosed method will result in a signal based on the characteristics of the labeled analyte or protein (for example, the mass-to-charge ratio). A complex analyte or protein sample can produce a unique pattern of signals. It is this pattern that can allow unique cataloging of analyte or protein samples and sensitive and powerful comparisons of the patterns of signals produced from different analyte or protein samples.

The presence, amount, presence and amount, or absence of different labeled analytes or different labeled proteins forms a pattern of signals that provides a signature or fingerprint of the analytes or proteins, and thus of the analyte or protein sample based on the presence or absence of specific analytes or analyte fragments (or protein or protein fragments) in the sample. For this reason, cataloging of this pattern of signals (that is, the pattern of the presence, amount, presence and amount, or absence of labeled analytes or proteins) is an embodiment of the disclosed method that is of particular interest.

Catalogs can be made up of, or be referred to, as, for example, a pattern of labeled analytes or proteins, a pattern of the presence of labeled analytes or proteins, a catalog of labeled analytes or proteins, or a catalog of analytes or proteins in a sample. The information in the catalog is preferably in the form of mass-to-charge information or compositional information. Catalogs can also contain or be made up of other information derived from the information generated in the disclosed method (for example, the identity of the analytes or proteins detected), and can be combined with information obtained or generated from any other source. The informational nature of catalogs produced using the disclosed method lends itself to combination and/or analysis or proteins using known bioinformatics systems and methods.

Such catalogs of analyte or protein samples can be compared to a similar catalog derived from any other sample to detect similarities and differences in the samples (which is indicative of similarities and differences in the analytes or proteins in the samples). For example, a catalog of a first analyte or protein sample can be compared to a catalog of a sample from the same type of organism as the first analyte or protein sample, a sample from the same type of tissue as the first analyte or protein sample, a sample from the same organism as the first analyte or protein sample, a sample obtained from the same source but at time different from that of the first analyte or protein sample, a sample from an organism different from that of the first analyte or protein sample, a sample from a type of tissue different from that of the first analyte or protein sample, a sample from a strain of organism different from that of the first analyte or protein sample, a sample from a species of organism different from that of the first analyte or protein sample, or a sample from a type of organism different from that of the first analyte or protein sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same analyte, or the same analyte sample. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

When comparing catalogs of analytes or proteins obtained from related samples, it is possible to identify the presence of a subset of correlated pairs of labeled analytes or proteins and their altered forms. The disclosed method can be used to detect the original labeled analytes or proteins (and determine characteristics of them) and the altered form of the labeled analytes or proteins. This pair of detected analytes or proteins will be characteristic of the analyte that is labeled and the specific multidimension signal used (although not necessarily unique).

Thus, multidimension signal labeling and multidimension signal protein labeling allows profiling of analytes and proteins, de novo discovery of analytes and proteins, and cataloging of analytes and proteins. The method has advantageous properties which can be used as a detection and analysis system for analyte and protein analysis, proteome analysis, proteomic, protein expression profiling, de novo analyte and protein discovery, finctional genomics, and analyte or protein detection.

Multidimension signals used and/or detected using different techniques (such as multidimension molecule labeling, reporter signal calibration, and multidimension signal fusions) can be used in and/or combined with MDSL.

C. Reporter Signal and Indicator Signal Calibration

In another form of the method, referred to as reporter signal calibration (RSC), a form of reporter signals referred to as reporter signal calibrators are mixed with analytes or analyte fragments (or protein or protein fragment), the reporter signal calibrators and the analytes or analyte fragments (or protein or protein fragment) are altered, and the altered forms of the reporter signal calibrators and altered forms of the analytes or analyte fragments (or protein or protein fragment) are detected. Reporter signal calibrators are useful as standards for assessing the amount of analytes or proteins present. That is, one can add a known amount of a reporter signal calibrator in order to assess the amount of analyte or protein present comparing the amount of altered analyte or analyte fragment (or protein or protein fragment) detected with the amount of altered reporter signal calibrator detected and calibrating these amounts with the known amount of reporter signal calibrator added (and thus the predicted amount of altered reporter signal calibrator).

The reporter signals and other multidimension signals used with them (such as indicator signal calibrators) can be subjected to an indicator level of analysis and a reporter signal level of analysis. Indicator signal calibrators can form a predetermined pattern with reporter signal calibrators when used together. In reporter signal calibration, reporter signal calibrators preferably share one or more common properties with one or more analytes while indicator signal calibrators preferably do not. Rather, the indicator signal calibrators serve to generate a pattern with the reporter signal calibrators.

The disclosed reporter signal calibration method generates, with high sensitivity, unique protein signatures related to the relative abundance of different proteins in tissue, microorganisms, or any other biological sample. The disclosed method allows one to define the status of a cell or tissue by identifying and measuring the relative concentrations of a small but highly informative subset of proteins. Such a measurement is known as a protein signature. Protein signatures are useful, for example, in the diagnosis, grading, and staging of cancer, in drug screening, and in toxicity testing.

In some embodiments, each analyte or analyte fragment (or protein or protein fragment) can share one or more common properties with at least one reporter signal calibrator such that the reporter signal calibrators and analytes or analyte fragments (or protein or protein fragment) having the common property can be distinguished and/or separated from other molecules lacking the common property.

In some embodiments, reporter signal calibrators and analytes and analyte fragments (or protein or protein fragment) can be altered such that the altered form of an analyte or analyte fragment (or protein or protein fragment) can be distinguished from the altered form of the reporter signal calibrator with which the analyte or analyte fragment (or protein or protein fragment) shares a common property. In some embodiments, the altered forms of different reporter signal calibrators can be distinguished from each other. In some embodiments, the altered forms of different analytes or analyte fragments (or protein or protein fragment) can be distinguished from each other.

In some embodiments of reporter signal calibration, the analyte or analyte fragment (or protein or protein fragment) is not altered and so the altered reporter signal calibrators and the analytes or analyte fragments (or protein or protein fragment) are detected. In this case, the analyte or analyte fragment (or protein or protein fragment) can be distinguished from the altered form of the reporter signal calibrator with which the analyte or analyte fragment shares a common property.

In some embodiments the analyte or analyte fragment (or protein or protein fragment) may be a reporter signal or a fragment of a reporter signal. In this case, the reporter signal calibrators serve as calibrators for the amount of reporter signal detected.

Note that when reporter signal calibration is used in connection with proteins and peptides, this form of reporter signal calibration is referred to as reporter signal protein calibration. Reporter signal protein calibration is useful, for example, for producing protein signatures of protein samples. As used herein, a protein signature is the presence, absence, amount, or presence and amount of a set of proteins or protein surrogates.

In some embodiments of reporter signal protein calibration, the presence of labile, scissile, or cleavable bonds in the proteins to be detected can be exploited. Peptides, proteins, or protein fragments (collectively referred to, for convenience, as protein fragments in the remaining description) containing such bonds can be altered by fragmentation at the bond. In this way, reporter signal calibrators having a common property (such as mass-to-charge ratio) with the protein fragments can be used and the altered forms of the reporter signal calibrators and the altered (that is, fragmented) forms of the protein fragments can be detected and distinguished. In this regard, although the protein fragments share a common property with their matching reporter signal calibrators, the altered forms of the reporter signal calibrators and altered forms of protein fragments can be distinguished (because, for example, the altered forms have different properties, such as different mass-to-charge ratios).

As an example of reporter signal protein calibration, a protein sample of interest can be digested with a serine protease, preferably trypsin. The digest generates a complex mixture of protein fragments. Among these protein fragments, there will exist a subset (approximately one protein fragment among every 400) that contains the dipeptide Asp-Pro. This dipeptide sequence is uniquely sensitive to fragmentation during mass spectrometry, and thus produces a high yield of ions in fragmentation mode. Since the human proteome consists of at least 2,000,000 distinct tryptic peptides, the number of protein fragments containing the Asp-Pro sequence is of the order of 5,000. Since some of these may exist as phosphopeptides or other modified forms, the number may be even higher. This number is sufficiently high to permit the selection of a subset (perhaps 50 to 100 or so) of fragmentable protein fragments that is suitable for generating a highly informative protein signature. Peptides that contain the Asp-Pro dipeptide sequence, peptides that contain amino acids that are modified by phosphorylation inside the cell, or peptides that contain an internal methionine are particularly preferred for use in reporter signal calibration. Alternatively, tryptic peptides terminating in arginine may be modified by reaction with acetylacetone (pentane-2,4-dione) to increase the frequency of fragment ions (Dikler et al., J Mass Spectrom 32:1337-49 (1997)). Selection of the subsets of protein fragments can be performed using bioinformatics in order to maximize the information content of the protein signatures.

For this form of reporter signal protein calibration, the protein digest can be mixed with a specially designed set of reporter signal calibrators, and then is analyzed using tandem mass spectrometry. In the case of a tandem in space instrument (for example, Q-Tof™ from Micromass), using first quadrupole settings for single-ion filtering (defined by the molecular mass of each unique fragment, which can be obtained from sequence data), followed by a collision stage for ion fragmentation, and finally TOF spectrometry of the peptide fragments (generated by cleavage at fragile bonds, such as Asp-Pro, bonds involving a phosphorylated amino acid, or bonds adjacent to an oxidized amino-acid such as methionine sulfoxide, Smith et aL, Free Radic Res. 26:103-11 (1997)) that arise from the original single-ion. In the second stage, signal to noise of the TOF measurement is much larger than in a conventional MS experiment. In general, one reporter signal calibrator can be used for each protein fragment in the sample that will be used to make up the protein signature (such protein fragments are referred to as signature protein fragments), and each is designed to fragment in an easily detectable pattern of masses, distinct from the fragment masses of the unfragmented signature protein fragments. The quadrupole filtering settings are then varied in sequence over a range of values (fifty, for example), corresponding to the masses of each of the protein fragments previously chosen to comprise the protein signature (that is, the signature protein fragments). At each filtered mass setting, there will be two types of signals detectable by TOF after fragmentation, one set derived from the tryptic peptide (that is, the original protein fragment), and another set corresponding to the reporter signal calibrator. The reporter signal calibrator permits one to calculate relative abundance for each of the signature protein fragments. These relative abundance ratios, determined for a given sample, constitute the protein signature. The detection limit of the tandem mass spectrometer in MS/MS mode, is remarkably good, perhaps of the order of 500 molecules of peptide. This level of detection is approximately 1,000 times better than that for MALDI-TOF mass spectrometry, and should permit the generation of protein signatures from single cells.

As can be seen, for this form of reporter signal calibration, the availability of the sequence of the entire human genome, as well as the genomes of many other organisms, can facilitate the identification of protein fragments that are unique in the context of all known proteins. That is, the sequence information can be used to identify peptides that will be generated in a protein signature and guide selection of reporter signal calibrators.

Multidimension signals used and/or detected using different techniques (such as multidimension molecule labeling, multidimension signal labeling, and multidimension signal fusions) can be used in and/or combined with RSC.

D. Multidimension Signal Fusions

In another form of the disclosed method and compositions, referred to as multidimension signal fusions (MDSF), multidimension signal peptides are joined with a protein or peptide of interest in a single amino acid segment, and the multidimension signal peptide, multidimension signal fusion, altered forms of the multidimension signal peptide, and/or altered forms of the multidimension signal fusion can be detected. Such fusions of proteins and peptides of interest with multidimension signal peptides can be expressed as a fusion protein or peptide from a nucleic acid molecule encoding the amino acid segment that constitutes the fusion. The fusion protein or peptide is referred to herein as a multidimension signal fusion. The multidimension signal peptides, a form of multidimension signal, allow for sensitive monitoring and detection of the proteins and peptides to which they are fused, and of expression of the genes, vectors, expression constructs, and nucleic acids that encode them. In particular, the multidimension signal fusions allow sensitive and multiplex detection of expression of particular proteins and peptides of interest, and/or of the genes, vectors, and expression constructs encoding the proteins and peptides of interest. The disclosed multidimension signal fusions can also be used for any purpose including as a source of multidimension signals for other forms of the disclosed method and compositions.

A “multidimension signal fusion,” refers to a protein, peptide, or fragment of a protein or peptide to which a multidimension signal peptide is fused (that is, joined by peptide bond(s) in the same polypeptide chain) unless the context clearly indicates otherwise. The multidimension signal fusion(s) can be fragmented, such as by protease digestion, prior to analysis. An expression sample to be analyzed can also be subjected to fractionation or separation to reduce the complexity of the samples. Fragmentation and fractionation can also be used together in the same assay. Such fragmentation and fractionation can simplify and extend the analysis of the expression.

The multidimension signal fusions can be produced by expression from nucleic acid molecules encoding the fusions. Thus, the disclosed fusions generally can be designed by designing nucleic acid segments that encode amino acid segments where the amino acid segments comprise a multidimension signal peptide and a protein or peptide of interest. A given nucleic acid molecule can comprise one or more nucleic acid segments. A given nucleic acid segment can encode one or more amino acid segments. A given amino acid segment can include one or more multidimension signal peptides and one or more proteins or peptides of interest. The disclosed amino acid segments consist of a single, contiguous polypeptide chain. Thus, although multiple amino acid segments can be part of the same contiguous polypeptide chain, all of the components (that is, the multidimension signal peptide(s) and protein(s) and peptide(s) of interest) of a given amino acid segment are part of the same contiguous polypeptide chain.

Thus, the disclosed method can use cells, cell lines, and organisms that have particular protein(s), gene(s), vector(s), and/or expression sequence(s) labeled (that is, associated with or involved in) multidimension signal fusions. For example, a set of nucleic acid constructs, each encoding a multidimension signal fusion with a different multidimension signal peptide, can be used to uniquely label a set of cells, cell lines, and/or organisms. Processing, in the disclosed method, of a sample from any of the labeled sources can result in a unique altered form of the multidimension signal peptide (or the amino acid segment or an amino acid subsegment) for each of the possible sources that can be distinguished from the other altered forms. Detection of a particular altered form identifies the source from which it came. As a more specific example, a genetically modified plant line (for example, a Roundup resistant corn line) into which a nucleic acid construct encoding a multidimension signal fusion has been introduced can be identified by detecting the multidimension signal fusion.

The disclosed multidimension signal fusions also are useful for creating cells, cell lines, and organisms that have particular protein(s), gene(s), vector(s), and/or expression sequence(s) labeled (that is, associated with or involved in) multidimension signal fusions. For example, a set of nucleic acid constructs, each encoding a multidimension signal fusion with a different multidimension signal peptide, can be used to uniquely label a set of cells, cell lines, and/or organisms. Processing of a sample from any of the labeled sources can result in a unique altered form of the multidimension signal peptide (or the amino acid segment or an amino acid subsegment) for each of the possible sources that can be distinguished from the other altered forms. Detection of a particular altered form identifies the source from which it came. As a more specific example, a nucleic acid construct encoding a multidimension signal fusion can be introduced into a genetically modified plant line (for example, a Roundup resistant corn line) and the plant line can then be identified by detecting the multidimension signal fusion. Preferred multidimension signal peptides for use in multidimension signal fusions used in or associated with different genes, proteins, vectors, constructs, cells, cell lines, or organisms would be those using differentially distributed mass. In particular, the use of alternative amino acid sequences using the same amino acid composition is preferred.

Nucleic acid sequences encoding multidimension signal peptides can be engineered into particular exons of a gene. This would be the normal situation when the gene encoding the protein to be fused contains introns, although sequence encoding a multidimension signal peptide can be split between different exons to be spliced together. Placement of nucleic acid sequences encoding multidimension signal peptides into particular exons is useful for monitoring and analyzing alternative splicing of RNA. The appearance of a multidimension signal peptide in the final protein indicates that the exon encoding the multidimension signal peptide was spliced into the mRNA.

The disclosed multidimension signal fusions also can be used to “label” particular pathways, regulatory cascades, and other suites of genes, proteins, vectors, and/or expressions sequences. Such labeling can be within the same cell, cell line, or organism, or across a set of cells, cell lines, or organisms. In one non-limiting example, the disclosed method can also be used to assess the state and/or expression of particular pathways, regulatory cascades, and other suites of genes, proteins, vectors, and/or expressions sequences. By using multidimension signal fusions to “label” such pathways, cascades, etc. within the same cell, cell line, or organism, or across a set of cells, cell lines, or organisms, the pathways, cascades and other systems can be assessed in a single assay and/or compared across cells, cell lines, or organisms. For example, nucleic acid segments encoding multidimension signal fusions can be associated with endogenous expression sequences of interest, endogenous genes of interest, exogenous expression sequences of interest, exogenous genes of interest, or a combination. The exogenous constructs then are introduced into the cells or organisms of interest. Thus, the expression of the genes and/or expression sequences assessed by detecting the multidimension signal peptides and/or multidimension signal fusions. The association with endogenous expression sequences or genes can be accomplished, for example, by introducing a nucleic acid molecule (encoding the multidimension signal fusion) for insertion at the site of the expression sequences or gene of interest, or by creating a vector or other nucleic acid construct (containing both the endogenous expression sequences or gene and a nucleic acid segment encoding the multidimension signal fusion) in vitro and introducing the construct into the cells or organisms of interest. Many other uses and modes of use are possible, a number of which are described in the illustrations below. The disclosed multidimension signal fusions can be used, for example, in any context and for any purpose that green fluorescent protein and green fluorescent protein fusions are used. However, the disclosed multidimension signal proteins have more uses and are more useful than green fluorescent protein at least because of the ability to multiplex more highly the disclosed multidimension signal fusions.

The multidimension signal peptides can be used for sensitive detection of one or multiple proteins (that is, the proteins to which the multidimension signal peptides are fused). In the method, proteins fused with multidimension signal peptides are analyzed using the multidimension signal peptides to distinguish the multidimension signal fusions. Detection of the multidimension signal peptides indicates the presence of the corresponding protein(s). The detected protein(s) can then be analyzed using known techniques. The multidimension signal fusions provide a unique protein/label composition that can specifically identify the protein(s). This is accomplished through the use of the specialized multidimension signal peptides as the labels.

In accordance with the invention, multidimension signal fusions can be fragmented, such as by protease digestion, prior to analysis. An expression sample to be analyzed can also be subjected to fractionation or separation to reduce the complexity of the samples. Fragmentation and fractionation can also be used together in the same assay. Such fragmentation and fractionation can simplify and extend the analysis of the expression.

Alteration of multidimension signals (e.g., reporter signal peptides) in multidimension signal fusions can produce a variety of altered compositions. Any or all of these altered forms can be detected. For example, the altered form of the multidimension signal peptide can be detected, the altered form of the amino acid segment (which contains the multidimension signal peptide) can be detected, the altered form of a subsegment of the amino acid segment can be detected, or a combination of these can be detected. Where the multidimension signal peptide is altered by fragmentation, the result generally will be a fragment of the multidimension signal peptide and an altered form of the amino acid segment containing the protein or peptide of interest and a portion of the multidimension signal peptide (that is, the portion not in the multidimension signal peptide fragment).

The protein or peptide of interest also can be fragmented. The result would be a subsegment of the amino acid segment. The amino acid subsegment would contain the multidimension signal peptide and a portion of the protein or peptide of interest. When the multidimension signal peptide in an amino acid subsegment is altered (which can occur before, during, or after fragmentation of the amino acid segment), the result is an altered form of the amino acid subsegment (and an altered form of the multidimension signal peptide). This altered form of amino acid subsegment can be detected. Where the multidimension signal peptide is altered by fragmentation, the result generally will be a fragment of the multidimension signal peptide and an altered form of (that is, fragment of) the amino acid subsegment. In this case, the altered form of the amino acid subsegment, which is also referred to herein as a multidimension signal fusion fragment, will contain a portion of the protein or peptide of interest and a portion of the multidimension signal peptide (that is, the portion not in the multidimension signal peptide fragment).

As with multidimension signals generally, multidimension signal fusions (also referred to as amino acid segments), multidimension signal fusion fragments (also referred to as subsegments of the multidimension signal fusions), or multidimension signal peptides can be used in sets where the multidimension signal fusions, multidimension signal fusion fragments, or multidimension signal peptides in a set can have one or more properties that generate a pattern in an indicator level of analysis. For example, the multidimension signal fusions, multidimension signal fusion fragments, or multidimension signal peptides in a set can have one or more common properties that allow the multidimension signal fusions, multidimension signal fusion fragments, or multidimension signal peptides to be separated or distinguished from molecules lacking the common property. In the case of multidimension signal fusions, amino acid segments and amino acid subsegments can be used in sets where the amino acid segments and amino acid subsegments in a set can have one or more properties that generate a pattern. For example, with multidimension signal fusions, amino acid segments and amino acid subsegments can be used in sets where the amino acid segments and amino acid subsegments in a set can have one or more common properties that allow the amino acid segments and amino acid subsegments, respectively, to be separated or distinguished from molecules lacking the common property. In general, the component(s) of the multidimension signal fusions having properties can depend on the component(s) to be detected and/or the mode of the method being used.

Nucleic acid molecules (or segments thereof) encoding multidimension signal fusions can be used in sets where the multidimension signal peptides in the multidimension signal fusions encoded by a set of nucleic acid molecules can have one or more properties that generate a pattern in an indicator level of analysis. Similarly, nucleic acid molecules (or segments thereof) encoding amino acid segments can be used in sets where the multidimension signal peptides in the amino acid segments encoded by a set of nucleic acid molecules (or segments thereof) can have one or more properties that generate a pattern. Nucleic acid molecules (or segments thereof) encoding amino acid segments can be used in sets where the amino acid segments encoded by a set of nucleic acid molecules can have one or more properties that allow the amino acid segments to be separated or distinguished from molecules lacking the common property. Other relationships between members of the sets of nucleic acid molecules, nucleic acid segments, amino acid segments, multidimension signal peptides, and proteins of interest are contemplated.

Multidimension signal peptides, such as reporter signal peptides, can be used in sets where the multidimension signal peptides in a set can have one or more common properties that allow the multidimension signal peptides to be separated or distinguished from molecules lacking the common property. In the case of multidimension signal fusions, amino acid segments and amino acid subsegments can be used in sets where the amino acid segments and amino acid subsegments in a set can have one or more common properties that allow the amino acid segments and amino acid subsegments, respectively, to be separated or distinguished from molecules lacking the common property. In general, the component(s) of the multidimension signal fusions having common properties can depend on the component(s) to be detected and/or the mode of the method being used.

Multidimension signal fusions can include other components besides a protein of interest and a multidimension signal peptide. For example, multidimension signal fusions can include epitope tags (e.g., his tag, myc tag, flu tag, or flag tag peptides) (see, for example, Brizzard et al. (1994) Immunoaffinity purification of FLAG epitope-tagged bacterial alkaline phosphatase using a novel monoclonal antibody and peptide elution. Biotechniques 16:730-735). Epitope tags can serve as tags by which multidimension signal fusions can be manipulated, isolated, separated, distinguished, associated, and/or bound. The use of epitope tags and flag peptides generally is known and can be adapted for use in the disclosed multidimension signal fusions.

In preferred embodiments, multidimension signal peptides, multidimension signal fusions (or amino acid segments), nucleic acid segments encoding multidimension signal fusion, and/or nucleic acid molecules comprising nucleic acid segments encoding multidimension signal fusions are used in sets where the multidimension signal peptides, the multidimension signal fusions, and/or subsegments of the multidimension signal fusions constituting or present in the set have similar properties (such as similar mass-to-charge ratios). The similar properties allow the multidimension signals, the multidimension signal fusions, or subsegments of the multidimension signal fusions to be distinguished and/or separated from other molecules lacking one or more of the properties. Preferably, the multidimension signals, the multidimension signal fusions, or subsegments of the multidimension signal fusions constituting or present in a set have the same mass-to-charge ratio (m/z). That is, the multidimension signals, the multidimension signal fusions, or subsegments of the multidimension signal fusions in a set are isobaric. This allows the multidimension signals, the multidimension signal fusions, or subsegments of the multidimension signal fusions to be separated precisely from other molecules based on mass-to-charge ratio. The result of the filtering is a huge increase in the signal to noise ratio (S/N) for the system, allowing more sensitive and accurate detection.

Cells, cell lines, organisms, and expression of genes and proteins can be detected using the disclosed multidimension signal fusions in a variety of ways. For example, the protein and attached multidimension signal peptide can be detected together, one or more peptides of the protein and the attached multidimension signal peptide(s) can be detected together, the fragments of the multidimension signal peptide can be detected, or a combination. Preferred detection involves detection of the multidimension signal fusion both before and after fragmentation of the multidimension signal peptide.

A preferred form of the disclosed method involves correlated detection of the multidimension signal peptides both before and after fragmentation of the multidimension signal peptide. This allows genes, proteins, vectors, and expression constructs “labeled” with a multidimension signal peptide to be detected and identified via the change in the multidimension signal fusion and/or multidimension signal peptide. That is, the nature of the multidimension signal fusion or multidimension signal peptide detected (non-fragmented versus fragmented) identifies the gene, protein, vector, or nucleic acid construct from which it was derived. Where the multidimension signal fusions and multidimension signal peptides are detected by mass-to-charge ratio, the change in mass-to-charge ratio between fragmented and non-fragmented samples provides the basis for comparison. Such mass-to-charge ratio detection is preferably accomplished with mass spectrometry.

As an example, a fusion between a protein of interest and a multidimension signal peptide designed as a mass spectrometry label can be expressed. The multidimension signal fusion can be subjected to tryptic digest followed by mass spectrometry of the resulting materials. A peak corresponding to the tryptic fragment containing the multidimension signal peptide will be detected. Fragmentation of the multidimension signal peptide in the mass spectrometer (preferably in a collision cell) would result in a shift in the peak corresponding to the loss of a portion of the attached multidimension signal peptide, the appearance of a peak corresponding to the lost fragment, or a combination of both events. Significantly, the shift observed will depend on which multidimension signal peptide is fused to the protein since different multidimension signal peptides will, by design, produce fragments with different mass-to-charge ratios. The combination event of detection of the parent mass-to-charge (with no collision gas) and the mass-to-charge corresponding to the loss of the fragment from the multidimension signal peptide (with collision gas) indicates a multidimension signal fusion (thus indicating expression of the multidimension signal fusion and of the gene, vector, or construct encoding it).

The multidimension signal fusions may be detected using mass spectrometry which allows sensitive distinctions between molecules based on their mass-to-charge ratios. A set of isobaric multidimension signal peptides or multidimension signal fusions can be used for multiplex labeling and/or detection of the expression of many genes, proteins, vectors, expression constructs, cells, cell lines, and organisms since the multidimension signal peptide fragments can be designed to have a large range of masses (or mass-to-charge ratios), with each mass (or mass-to-charge ratio) individually distinguishable upon detection. Further, use of more than one isobaric multidimension signal set where the sets are not isobaric to each other allows both generation of predetermined patterns and a powerful means to increase the multiplexing potential of the disclosed methods.

Where the same gene, protein, vector, expression construct, cell, cell line, or organism (or the same type of gene, protein, vector, expression construct, cell, cell line, or organism) is labeled with a set of multidimension signal fusions that are isobaric or that include isobaric multidimension signal peptides (by, for example, “labeling” the same gene, protein, vector, expression construct, cell, cell line, or organism in different samples), the set of multidimension signal fusions or multidimension signal peptides that results will also be isobaric. Fragmentation of the multidimension signal peptides will split the set of multidimension signal peptides into individually detectable multidimension signal fusion fragments and multidimension signal peptide fragments of characteristically different mass.

Analogously, non-isobaric multidimension signals and sets of multidimension signals that are not isobaric to the other sets can be used to label the same gene, protein, vector, expression construct, cell, cell line, or organism (or the same type of gene, protein, vector, expression construct, cell, cell line, or organism) (by, for example, labeling the same gene, protein, vector, expression construct, cell, cell line, or organism in different samples). The result will be sets of multidimension signal fusions or multidimension signal peptides that are not isobaric; a pattern of multidimension signal fusions or multidimension signal peptides having different masses will be generated. Use of combinations of isobaric and non-isobaric multidimension signals or sets of multidimension signals to label the same gene, protein, vector, expression construct, cell, cell line, or organism in different samples can generate a pattern of masses in an indicator level of analysis. Fragmentation of the isobaric multidimension signals in a reporter signal level of analysis will split the set of multidimension signal fusions or multidimension signal peptides into individually detectable labeled proteins of characteristically different mass.

Multidimension signals used and/or detected using different techniques (such as multidimension molecule labeling, multidimension signal labeling, and reporter signal calibrators) can be used in and/or combined with MDSF.

Some forms of the method can involve labeling analytes or proteins in a first sample or a first set of samples with one or more isobaric multidimension signals or one or more sets of isobaric multidimension signals, labeling analytes in a second sample or second set of samples with one or more different multidimension signals or one or more different sets of multidimension signals, mixing the first and second samples to form an analysis sample, analyzing the multidimension signal-labeled analytes in the analysis sample to identify one or more predetermined patterns that result from the multidimension signals, where identification of the one or more predetermined patterns identifies one or more portions of the analysis sample, analyzing the multidimension signals in one or more of the one or more identified portions of the analysis sample to identify the multidimension signals present in identified portion of the analysis sample, where analyzing the multidimension signals in one or more of the one or more identified portions of the analysis sample is accomplished by fragmentation of the multidimension signals in the identified portion to produce multidimension signal fragments having different masses, and detection of the different multidimension signal fragments based on their mass-to-charge ratios. In some forms of the method, one or more of the sets of multidimension signals can be a set of reporter signals and the analysis of the multidimension signals in one or more of the one or more identified portions of the analysis sample identifies the reporter signals. One or more of the sets of multidimension signals can include, for example, both reporter signals and indicator signals, a set of reporter signals and an indicator signal, a reporter signal and a set of indicator signals or a set of reporter signals and a set of indicator signals. For example, the method may be carried out using a tandem mass spectrometer as described elsewhere herein.

Nucleic acid sequences and segments encoding multidimension signal fusions can be expressed in any suitable manner. For example, the disclosed nucleic acid sequences and nucleic acid segments can be expressed in vitro, in cells, and/or in cells in organism. Many techniques and systems for expression of nucleic acid sequences and proteins are known and can be used with the disclosed multidimension signal fusions. For example, many expression sequences, vector systems, transformation and transfection techniques, and transgenic organism production methods are known and can be used with the disclosed multidimension signal peptide method and compositions.

For example, kits for the in vitro transcription/translation of DNA constructs containing promoters and nucleic acid sequence to be transcribed and translated are known (for example, PROTEINscript-PRO™ from Ambion, Inc. Austin Tex.; Wilkinson (1999) “Cell-Free And Happy: In Vitro Translation And Transcription/Translation Systems”, The Scientist 13[13]:15, Jun. 21, 1999). Such constructs can be used in the genomic DNA of an organism, in a plasmid or other vector that may be transfected into an organism, or in in vitro systems. For example, constructs containing a promoter sequence and a nucleic acid sequence that, following transcription and translation, results in production of green fluorescence protein or luciferase as a multidimension/marker in in vivo systems are known (for example, Sawin and Nurse, “Identification of fission yeast nuclear markers using random polypeptide fusions with green fluorescent protein.” Proc Natl Acad Sci U S A 93(26): 15146-51 (1996); Chatterjee et al., “In vivo analysis of nuclear protein traffic in mammalian cells.” Exp Cell Res 236(1):346-50 (1997); Patterson et al., “Quantitative imaging of TATA-binding protein in living yeast cells.” Yeast 14(9):813-25 (1998); Dhandayuthapani et al., “Green fluorescent protein as a marker for gene expression and cell biology of mycobacterial interactions with macrophages.” Mol Microbiol 17(5):901-12 (1995); Kremer et al., “Green fluorescent protein as a new expression marker in mycobacteria.” Mol Microbiol 17(5):913-22 (1995); Reiländer et al., “Functional expression of the Aequorea victoria green fluorescent protein in insect cells using the baculovirus expression system.” Biochem Biophys Res Commun 219(1): 14-20 (1996); Mankertz et al., “Expression from the human occludin promoter is affected by tumor necrosis factor alpha and interferon gamma” J Cell Sci, 113:2085-90 (2000); White et al., “Real-time analysis of the transcriptional regulation of HIV and hCMV promoters in single mammalian cells” J Cell Sci, 108:441-55 (1995)). Green fluorescence protein, or variants, have been shown to be stably incorporated and not interfere with the organism—generally GFP is larger relative to the disclosed multidimension signal peptides (GFP from Aequorea Victoria is 238 amino acids in size; NCBI GI:606384), and thus the generally smaller multidimension signal peptides are less likely to disrupt an expression system to which they are added.

Techniques are known for modifying promoter regions such that the endogenous promoter is replaced with a promoter-multidimension construct, for example, where the multidimension is green fluorescent protein (Patterson et al., “Quantitative imaging of TATA-binding protein in living yeast cells.” Yeast 14(9): 813-25 (1998)) or luciferase. Transcription factor concentrations are followed by monitoring the GFP or luciferase. These techniques can be used with the disclosed multidimension signal fusions and multidimension signal fusion constructs. Techniques are also known for targeted knock-in of nucleic acid sequences into a gene of interest, typically under control of the endogenous promoter. Such techniques, which can be used with the disclosed method and compositions, have been used to introduce multidimension/markers of the transcription and translation of the gene where the nucleic acid was inserted. The same techniques can be used to place the disclosed multidimension signal fusions under control of endogenous expression sequences. Alternately, non-targeted knock-ins (techniques for which are also known; Hobbs et al. “Development of a bicistronic vector driven by the human polypeptide chain elongation factor 1 alpha promoter for creation of stable mammalian cell lines that express very high levels of recombinant proteins” Biochem Biophys Res Commun, 252:368-72 (1998); Kershnar et al., “Immunoaffinity purification and functional characterization of human transcription factor IIH and RNA polymerase II from clonal cell lines that conditionally express epitope-tagged subunits of the multiprotein complexes” J Biol Chem, 273:34444-53 (1998); Wu and Chiang, “Establishment of stable cell lines expressing potentially toxic proteins by tetracycline-regulated and epitope-tagging methods” Biotechniques 21:718-22, 724-5 (1996)) can be used to follow the level or activity of transcription factors—multidimension signal peptide fusions associated with the inserted nucleic acid code can directly indicate the transcription/translation activity.

The disclosed multidimension signal fusions also can be used in the detection and analysis of protein interactions with other proteins and molecules. For example. interaction traps for protein-protein interactions include the well known yeast two-hybrid (Fields and Song, “A novel genetic system to detect protein-protein interactions” Nature 340:245-6 (1989); Uetz et al., “A comprehensive analysis of protein-protein interactions in Saccharomyces cerevisiae” Nature 403:623-7 (2000)) and related systems (Ausubel et al., Current Protocols in Molecular Biology, John Wiley & Sons, Inc., 2001; Van Criekinge and Beyaert, “Yeast two-hybrid: state of the art” Biological Procedures Online, 2(1), 1999). Incorporation of nucleic acid sequence encoding a peptide multidimension signal can be introduced into these systems, for example at a terminus of the ordinarily used LacZ selection region (LacZ selection is described in, for example, Sambrook et al., Molecular Cloning: A Laboratory Manual, second edition, 1989, Cold Spring Harbor Laboratory Press, New York). A set of such incorporated sequences (for example, in a set of such plasmids, where each plasmid has a multidimension signal coding sequence and the LacZ functionality), allows the unambiguous detection of many interactions simultaneously rather (as many different interactions as multidimension signals used).

In another mode of multidimension signal fusions, a nucleic acid sequence encoding a multidimension signal could be added to sequence encoding the constant (C) region of T cell and B cell receptors. The multidimension signal would appear in T or B cell receptors when that C region is spliced to a J region following transcription.

In another mode of multidimension signal fusions, referred to as multidimension signal presentation, the presentation of specific antigenic peptides by major histocompatibility (MHC) and non-major histocompatibility molecules can be detected and analyzed. It is well known that protein antigens are processed by antigen presenting cells and that small peptides, typically 8-12 amino acids are presented by Class I and Class II MHC molecules for recognition by T cells. The study of specific T cell/peptide-MHC complexes is technically challenging due various labeling requirements (either radioactive or fluorescence) and the common reliance on antibody reagents that recognize specific receptors and/or peptide-MHC complexes.

There is a need to be able to further expand our knowledge of antigen processing and antigen presentation. Multidimension signals that have been engineered into specific protein antigens could provide novel insight into this process and enable new experimental approaches. For instance, consider two viral or bacterial proteins, protein A and protein B, that differ by only a few amino acids. It would be useful to know if they are processed and presented to immune cells (for example, T cells) with the same efficiency. By engineering multidimension signals into protein A and engineered protein B to antigen presenting cells, one could test for the presence of the different multidimension signals presented on and thus determine if the proteins are efficiently processed and presented. The presence of multidimension signal A (present in protein A) but not multidimension signal B (present in protein B), indicates that protein A is processed and that protein B is not. The lack of antigen processing of protein B may then be an explanation of why a virus or bacteria escapes immune surveillance by the immune system. Antigenic peptides are characterized by conserved anchor residues near both the amino and carboxy ends, with more heterogeneity tolerated in the middle. This middle heterogeneity is thus a preferred site for addition of a multidimension signal peptide.

Preferred multidimension signal peptides for use in multidimension signal fusions used in or associated with different genes, proteins, vectors, constructs, cells, cell lines, or organisms would be those using differentially distributed mass. In particular, the use of alternative amino acid sequences using the same amino acid composition is preferred.

Multidimension signal fusions can be used to monitor and analyze alternative RNA splicing. A central problem in translating the information in the genome to protein expression is an understanding of MRNA alternative processing, and the generation of protein isoforms via alternative exon utilization (Black, “Protein diversity from alternative splicing: a challenge for bioinformatics and post-genome biology” Cell 103:367-70 (2000)). Many examples of the use of alternative pre-mRNA splicing to generate protein isoform diversity exist, such as in the control of erythroid differentiation (see, for example, Hou and Conboy, “Regulation of alternative pre-mRNA splicing during erythroid differentiation” Curr Opin Hematol 8:74-9 (2001)). Often the detection of complex, alternatively spliced protein isoforms is a difficult task, since exons may be as small as 6 amino acids in protein of over 2000 amino acids (see, for example, Cianci et al., “Brain and muscle express a unique alternative transcript of a II spectrin” Biochem 38:15721-15730 (1999)).

Exon utilization and processing information can be obtained by insertion of a nucleic acid sequence encoding a multidimension signal into the exon sequence of interest (thus forming a nucleic acid segment that encodes a multidimension signal fusion). The insertions can be made, for example, into genomic DNA, appropriate mini-gene constructs, or non-endogenous pre-mRNA introduced into the cell. Use of a set of multidimension signals allows the multiplexed readout of all exons of a translated protein at one time. The use of mini-gene constructs or constructs incorporating short exogenous open-reading frame DNA sequences into exons, and the incorporation of foreign DNA in association with functional intron splice elements are developed technologies that can be used for incorporation of multidimension signals (see, for example, Gee et al., “Alternative splicing of protein 4.1R exon 16: ordered excision offlanking introns ensures proper splice site choice” Blood 95:692-9 (2000); Kikumori et al., “Promiscuity of pre-mRNA spliceosome-mediated trans splicing: a problem for gene therapy?“Hum Gene Ther 12:1429-41 (2001); Malik et al., “Effects of a second intron on recombinant MFG retroviral vector” Arch Virol 146:601-9 (2001); Virts and Raschke, “The role of intron sequences in high level expression from CD45 cDNA constructs” J Biol Chem 276:19913-20 (2001)). Detection of the multidimension signals, the amounts of the multidimension signals, and the knowledge of which multidimension signal correlates with which exon, provides information about exon usage and alternative splicing.

E. Lipid Multidimension Signals

The disclosed method and compositions also can be used to monitor lipid composition, distribution, and processing. Lipids are hydrophobic biomolecules that have high solubility in organic solvents. They have a variety of biological roles that make them valuable targets for monitoring. As a nutritional source, lipids (together with carbohydrates) constitute an important source of cellular energy and metabolic intermediates needed for cell signaling and other processes. Lipids processed for energy conversion typically pass through a variety of enzymatic pathways, generating many intermediates. A summary of these cycles is available in most modem biochemistry texts (see, for example, Stryer, 1995). Monitoring the processing of acyl chain intermediates as they are metabolized is an important tool in lipid and cell biological research, as well as for the clinical detection of biochemical diseases such as medium-chain acyl-CoA dehydrogenase deficiencies (see, for example, Zschocke et al., “Molecular and functional characterization of mild MCAD deficiency.”, Hum Genet 108:404-8 (2001)). Incorporating multidimension signals into, or associating multidimension signals with, lipids can improve methods of detecting lipids (such as Andresen et al., “Medium-chain acyl-CoA dehydrogenase (MCAD) mutations identified by MS/MS-based prospective screening of newborns differ from those observed in patients with clinical symptoms: identification and characterization of a new, prevalent mutation that results in mild MCAD deficiency” Am J Hum Genet 68:1408-18. (2001)) by allowing, for example, more rapid and multiplex detection of processed acyl chain intermediates.

In another role, lipids finction as the most fundamental and defining component of all biological membranes. The three major types of membrane lipids are phospholipids, glycolipids, and cholesterol. The most abundant of these are the phospholipids, derived either from glycerol or sphingosine. Those based on glycerol typically contain two esterified long-chain fatty acids (14 to 24 carbons) and a phosphorylated alcohol or sugar. Phospholipids based on sphingosine contain a single fatty acid. Collectively these lipids contribute to the structure and fluidity of biological membranes. Cyclic changes in their processing, particularly of acidic glycophosolipids such as phosphatidyl inositol 4,5 phosphate, also regulate a wide variety of cellular processes (see, for example, Cantrell, “Phosphoinositide 3-kinase signaling pathways” J Cell Sci 114:1439-45 (2001); Payrastre et al., “Phosphoinositides: key players in cell signaling, in time and space” Cell Signal 13:377-87 (2001)). Thus, by incorporating multidimension signals into, or associating multidimension signals with, the acyl chains of such molecules, the subsequent incorporation of such multidimension molecules into either in vitro assays such as those used for enzyme determinations or in vivo assays, allows one to rapidly follow the segregation of these lipids into distinct cellular compartments (for example, golgi versus plasma membrane (see, for example, Godi et al., “ARF mediates recruitment of PtdIns-4-OH kinase-beta and stimulates synthesis of PtdIns(4,5)P2 on the Golgi complex” Nat Cell Biol 1:280-7 (1999)), and their processing via metabolic and signaling pathways such as those cited above.

It is known that exogenous lipid labels can be incorporated readily into biological systems, and the disclosed multidimension signals also can be incorporated into such systems. For example, spin-labeled acyl fatty acids and phospholipids have been incorporated into the membranes of phospholipid vesicles and cells (see, for example, Komberg and McConnell, “Inside-outside transitions of phospholipids in vesicle membranes” Biochemistry 10:1111-20 (1971); Komberg and McConnell, “Lateral diffusion of phospholipids in a vesicle membrane” Proc Natl Acad Sci USA 68:2564-8 (1971); Arora et al., “Selectivity of lipid-protein interactions with trypsinized Na, K-ATPase studied by spin-label EPR” Biochim Biophys Acta 1371:163-7 (1998); Alonso et aL, “Lipid chain dynamics in stratum corneum studied by spin label electron paramagnetic resonance” Chem Phys Lipids 104:101-11 (2000)).

Triglycerides, or the acyl chain of sphinoglipids or glycolipids, and cholesterol, may be synthesized to include a multidimension signal. An example of such a multidimension signal would be a lipid made from an aliphatic chain with a carboxylic acid with a photocleavable bond. Examples of photocleavable bonds are described by Glatthar and Geise, Org. Lett, 2:2315-2317 (2000); Guillier et al., Chem. Rev. 100:2091-2157 (2000); Wierenga, U.S. Pat. No. 4,086,254; and elsewhere here. A set of multidimension signals may be prepared by locating the cleavable bond at different locations within an aliphatic chain (thus resulting in fragments of different mass when the bond is cleaved). The aliphatic chain with a photocleavable bond constitutes the multidimension signal. Such synthetic multidimension molecules can be incorporated into synthetic triglycerides by, for example, a dehydration reaction. Once formed, a set of these synthetic triglycerides can be introduced into biological systems of interest, such as those described above. Multidimension signals can be recovered from the biological system of interest for detection and quantitation by, for example, extraction of the lipid into chloroform and release of multidimension signals from the trigyceride using a lipase or hydrolysis reaction.

F. Sensitive Coded Detection Systems

Multidimension signals, such as reporter signals and indicator signals, can be used as blocks in the detector systems described in U.S. Application Publication US-2003-0124595-A1, the contents of which are incorporated herein by reference. The detector systems can be referred to as Sensitive Coded Detection Systems (SCDS). Sets of multidimension signals, such as sets of reporter signals can be used as block groups in SCDS. U.S. Application Publication US-2003-0124595-A1 describes SCDS, including compositions, referred to as detectors, that are based on the use of carriers comprising a set of arbitrary molecular tags that have been optimized to facilitate a subsequent detection. The molecular tags are referred to as blocks and the set of blocks is referred to as a block group. The carriers are linked, preferably by covalent coupling, to specific recognition molecules. The specific recognition molecules are referred to as specific binding molecules. The detectors, by virtue of the directly or indirectly linked recognition molecules, may be used as reporters in bioassays. The blocks can be optimized by their chemical composition, so that they may be efficiently separated by, for example, mass spectrometry. Blocks to be separated by mass spectrometry will differ in molecular weight, preferably by well resolved mass (or mass-to-charge ratio) differences that allow for reliable separation. For separation by mass spectrometry, the carriers can be loaded with reporter signals where differences between the mass-to-charge ratios of altered forms of the reporter signals can be used to distinguish and detect the carriers.

U.S. Application Publication US-2003-0124595-A1 also describes SCDS methods of detecting multiple analytes in a sample in a single assay by encoding target molecules with signals followed by decoding of the encoded signal (using detectors with block groups). This encoding/decoding uncouples the detection of a target molecule from the chemical and physical properties of the target molecule. In basic form, the method involves association of one or more detectors with one or more target samples—where the detector comprises a specific binding molecule, a carrier, and a block group composed of blocks—and detection of the block groups via detection of the blocks. The detectors associate with target molecules in the target sample(s) via the specific binding molecule. Generally, the detectors correspond to one or more target molecules, and the block groups correspond to one or more detectors. Thus, detection of particular block groups indicates the presence of the corresponding detectors. In turn, the presence of particular detectors indicates the presence of the corresponding target molecules.

This indirect detection in SCDS uncouples the detection of target molecules from the chemical and physical properties of the target molecules by interposing block groups that essentially can have any arbitrary chemical and physical properties. In particular, block groups (and the blocks of which they are composed) can have specific properties useful for detection, and block groups and blocks within an assay can have highly ordered or structured relationships with each other. It is the (freely chosen) properties of the block groups and blocks, rather than the (take them as they are) properties of the target molecules that matters at the point of detection.

The multidimension signals, reporter signals, indicator signals, sets of multidimension signals, sets of reporter signals, and sets of indicators signals can be chosen such that the blocks in block groups, detectors or groups of detectors can generate predetermined patterns as described herein. For example, a set of reporter signals can be used with a set of indicator signals, two sets of reporter signals can be used together, and a set of reporter signals can be used with a single indicator signal. Detection, analysis and use of predetermined patterns as described herein can be used in the detection, analysis and use of the disclosed multidimension signals when used in detectors and other SCDS components and methods described in U.S. Application Publication US-2003-0124595-A1. Detectors, block groups, blocks, identity composition and amount composition are defined in U.S. Application Publication US-2003-0124595-A1, which definitions are hereby incorporated by reference.

Thus, the invention provides detectors with one or more target samples, wherein the detectors each comprise a specific binding molecule, a carrier, and a block group, wherein the block group comprises blocks, wherein the blocks comprise a set of reporter signals and one or more indicator signals (and/or two or more sets of reporter signals). The reporter signals in each set can have a common property, wherein the common property can allow the reporter signals to be distinguished or separated from molecules lacking the common property, wherein the reporter signals can be altered, wherein the altered forms of each reporter signal can be distinguished from every other altered form of reporter signal. The reporter signals and one or more of the indicator signals (or two or more of the sets of reporter signals) will generate a predetermined pattern under conditions where the common property allows the reporter signals to be distinguished and/or separated from molecules lacking the common property. In some forms, the indicator signals do not have the common property. The common property can be mass-to-charge ratio, wherein the reporter signals can be altered by altering their mass, wherein the altered forms of the reporter signals can be distinguished via differences in the mass-to-charge ratio of the altered forms of reporter signals. The mass of the reporter signals can be altered by fragmentation. Alteration of the reporter signals also can alter their charge.

The blocks can have the same amount composition, but the blocks need not all have the same amount composition. A plurality of detectors can be associated with the one or more target samples, wherein the block group of each detector can have a different composition of blocks. Each block group can have the same number of blocks, but the block groups need not all have the same number of blocks. Each block group can have a different identity composition of blocks. Block groups that have the same identity composition of blocks can have different amount compositions of blocks. Detectors, block groups, blocks, identity composition and amount composition are defined in U.S. Application Publication US-2003-0124595-A1, which definitions are hereby incorporated by reference.

The blocks can be capable of being detected through MALDI-TOF spectroscopy. The blocks can be isobaric blocks. A plurality of detectors can be associated with one or more target samples, wherein the blocks of each detector can be different. All of the blocks of all of the detectors can have the same mass-to-charge ratio. The blocks can be altered by altering their mass, charge, or both, wherein the altered forms of the blocks can be distinguished via differences in the mass-to-charge ratio of the altered forms of the blocks.

The carrier can be selected from the group consisting of beads, liposomes, microparticles, nanoparticles, and branched polymer structures. The carrier can be a bead. The carrier can be a liposome or microbead. The liposomes can be unilamellar vesicles. The vesicles can have an average diameter of 150 to 300 nanometers. The liposome can have an internal diameter of 200 nanometers. The carrier can be a dendrimer. The dendrimer can be contacting a macromolecule selected from the group consisting of DNA, RNA, and PNA. The macromolecule can be an oligonucleotide between 20 and 300 nucleotides in length.

The specific binding molecule can be selected from the group consisting of antibodies, ligands, binding proteins, receptor proteins, haptens, aptamers, carbohydrates, synthetic polyamides, and oligonucleotides. The specific binding molecule can be a binding protein. The binding protein can be a DNA binding protein. The DNA binding protein can contain a motif selected from the group consisting of a zinc finger motif, leucine zipper motif, and helix-turn-helix motif.

The specific binding molecule can be an oligonucleotide. The oligonucleotide can be between 10 and 40 nucleotides in length, or can be between 16 and 25 nucleotides in length. The oligonucleotide can be a peptide nucleic acid. The oligonucleotide can form a triple helix with the target sequence. The oligonucleotide can comprise a psoralen derivative capable of covalently attaching the oligonucleotide to the target sequence.

The specific binding molecule can be an antibody, such an antibody that can bind a protein. The blocks can be oligonucleotides, carbohydrates, synthetic polyamides, peptide nucleic acids, antibodies, ligands, proteins, haptens, zinc fingers, aptamers, mass labels, or any combination of these. The specific binding molecule and the carrier can be covalently linked. The carrier and the blocks can be covalently linked. The specific binding molecule and the carrier can be covalently linked. The specific binding molecule can comprise a first oligonucleotide and the carrier can comprise a second oligonucleotide which can hybridize to the first oligonucleotide. The first oligonucleotide can be conjugated to an antibody which binds a protein.

Also disclosed is a composition for detecting an analyte comprising a specific binding molecule, a carrier, and a block group, wherein the block group comprises blocks, and wherein the blocks comprise a set of reporter signals and one or more indicator signals (and/or two or more sets of reporter signals). The reporter signals in a set can have a common property, wherein the common property can allow the reporter signals to be distinguished or separated from molecules lacking the common property, wherein the reporter signals can be altered, wherein the altered forms of each reporter signal can be distinguished from every other altered form of reporter signal. The reporter signals and one or more of the indicator signals (or two or more of the sets of reporter signals) will generate a predetermined pattern under conditions where the common property allows the reporter signals to be distinguished and/or separated from molecules lacking the common property. In some forms, the indicator signals do not have the common property.

G. Rearranging Multidimension Signals

Another embodiment of the disclosed method and compositions, referred to as rearranging multidimension signals (rearranging MDS or RMDS), enables one to detect the occurrence of specific gene rearrangement events, their protein products, and specific cell populations bearing those receptors. RMDS will also allow one to follow the progression or development of certain receptors and cells or populations of cells by monitoring the presence and/or absence of a multidimension signal. Design considerations for rearranged multidimension signals are analogous to those required for multidimension signal fusions as described elsewhere herein.

Most embodiments of the disclosed method involve intact multidimension signals that are associated with analytes in various ways. RMDS make use of processes, such as biological processes, to form multidimension signals by specific rearrangement of the multidimension signal pieces or rearrangement of nucleic acid segments encoding only portions of multidimension signals. One form of RMDS utilizes endogenous biological systems, such as the variable-diversity-joining (V-D-J) gene rearrangement machinery present in the mammalian immune system. In this system, short stretches of germline DNA (the V, D & J gene fragments) that are not contiguous, are brought together (recombined) prior to serving as a template for transcription. Gene rearrangement occurs in white blood cells such as T and B lymphocytes and is a key mechanism for generating diversity of T cell and B cell antigen receptors. Theoretically, billions of different receptors can be generated. This level of complexity makes it difficult to detect the presence of rare rearrangement events, or receptors. PCR based assays and flow cytometry approaches are now used to study receptor diversity. However, PCR approaches are laborious and do not provide any information on the status of expressed protein. Flow cytometry approaches have limited multiplexing capabilities due to emission spectra overlap of the fluorescent probes used.

If one desired to test for 50-100 T cell or B cell receptors, one would need to make use of a similar number of antibodies to those receptors, something that in practice is not done. Therefore, there is a real need for methods that would allow highly sensitive and specific detection of specific receptors in a highly complex pool of receptors. The ability to highly multiplex this approach would enable currently unattainable experimental approaches. The disclosed multidimension signal technology allows large scale multiplexing of signals for detection.

As an example of RMDS, transgenic mice can be generated in which nucleic acid sequences encoding multidimension signals have been engineered into the mouse germline. Methods for doing this are well known in the art and include using standard molecular biology methods to engineer rearranging multidimension signal into, for example, yeast or bacterial artificial chromosomes (YACs or BACs) and then using these constructs to generate transgenic mice.

As an example of the use of immunoglobulin rearrangement for RMDS, part of a multidimension signal could be encoded on the D region and another part of the multidimension signal could be encoded on the J region. Upon a rearrangement event that joined the D and J regions encoding these “partial” multidimension signals, a coding sequence for a “complete” multidimension signal would be generated. Following transcription and translation, the multidimension signal would be encoded within the protein product. The multidimension signal could then be detected as described elsewhere herein. In the absence of a rearrangement event that joins the engineered D and J region, no multidimension signal would be detected. By including sequences encoding parts of a variety of multidimension signals with different D and J regions, a variety of different multidimension signals can be generated by rearrangement, a different, and diagnostic, multidimension signal for each of the different possible rearrangements. This system also could be extended to include, for example, multidimension signals split among three or more gene regions (for example, V-D-J, V-D-D-J, etc) with the result that multiple rearrangement events would produce the multidimension signal. In this mode, the combinations of rearrangements of the multidimension signal parts can give rise to a large number of different multidimension signals, each characterized by the specific multidimension signal parts rearranged to form the multidimension signal.

Transgenic mice carrying RMDS would enable one to address questions that would otherwise be very difficult or impossible to address. For instance, one could dissect what specific T and B cell receptors (out of the thousands or millions possible) respond to specific stimuli or what cell types are present at certain stages of development.

Transgenic mice carrying rearranging multidimension signals would enable one to address questions that would otherwise be very difficult or impossible to address. For instance, one could dissect what specific T and B cell receptors (out of the thousands or millions possible) respond to specific stimuli or what cell types are present at certain stages of development.

H. Mass Spectrometers

The disclosed methods can make use of mass spectrometers for analysis of multidimension signals, altered forms of multidimension signals, and various analytes and analyte fragments. Mass spectrometers are generally available and such instruments and their operations are known to those of skill in the art. Fractionation systems integrated with mass spectrometers are commercially available, exemplary systems include liquid chromatography (LC) and capillary electrophoresis (CE).

The principle components of a mass spectrometer include: (a) one or more sources, (b) one or more analyzers and/or cells, and (c) one or more detectors. Types of sources include Electrospray Ionization (ESI) and Matrix Assisted Laser Desorption Ionization (MALDI). Types of analyzers and cells include quadrupole mass filter, hexapole collision cell, ion cyclotron trap, and Time-of-Flight (TOF). Types of detectors include Multichannel Plates (MCP) and ion multipliers. A preferred mass spectrometer for use with the disclosed method is described by Krutchinsky et al., Rapid Automatic Identification of Proteins Utilizing a Novel MALDI-Ion Trap Mass Spectrometer, Abstract of the 49^{th} ASMS Conference on Mass Spectrometry and Allied Topics (May 27-31, 2001), The Rockefeller University, New York, N.Y.

Mass spectrometers with more than one analyzer/cell are known as tandem mass spectrometers. There are two types of tandem mass spectrometers, as well as hybrids and combinations of these types: “tandem in space” spectrometers and “tandem in time” spectrometers. Tandem mass spectrometers where the ions traverse more than one analyzer/cell are known as tandem in space mass spectrometers. Tandem in space spectrometers utilize spatially ordered elements and act upon the ions in turn as the ions pass through each element. Tandem mass spectrometers where the ions remain primarily in one analyzer/cell are known as tandem in time mass spectrometers. Tandem in time spectrometers utilize temporally ordered manipulations on the ions as the ions are contained in a space. Hybrid systems and combinations of these types are known. The ability to select a particular mass-to-charge ratio of interest in a mass analyzer is typically characterized by the resolution (reported as the centroid mass-to-charge divided by the full width at half maximum of the selected ions of interest). Thus resolution is an indicator of the narrowness of the ion mass-to-charge distribution passed through the analyzer to the detector. Reference to such resolution is generally noted herein by referring to the ability of a mass spectrometer to pass only a narrow range of mass-to-charge ratios.

A preferred form of mass spectrometer for use in the disclosed methods is a tandem mass spectrometer, such as a tandem in space tandem mass spectrometer. As an example of the use of a tandem in space class of instrument, the isobaric multidimension signals can be first passed through a filtering quadrupole, the multidimension signals are fragmented (preferably in a collision cell), and the fragments are distinguished and detected in a time-of-flight (TOF) stage. In such an instrument the sample is ionized in the source (for example, in a MALDI ion source) to produce charged ions. It is preferred that the ionization conditions are such that primarily a singly charged parent ion is produced. A first quadrupole, Q0, is operated in radio frequency (RF) mode only and acts as an ion guide for all charged particles. The second quadrupole, Q1, is operated in RF+DC mode to pass only a narrow range of mass-to-charge ratios (that includes the mass-to-charge ratio of the multidimension signals). This quadrupole selects the mass-to-charge ratio of interest. Quadrupole Q2, surrounded by a collision cell, is operated in RF only mode and acts as ion guide. The collision cell surrounding Q2 can be filled to appropriate pressure with a gas to fracture the input ions by collisionally induced dissociation when fragmentation of the multidimension signals is desired. The collision gas preferably is chemically inert, but reactive gases can also be used. Preferred molecular systems utilize multidimension signals that contain scissile bonds, labile bonds, or combinations, such that these bonds will be preferentially fractured in the Q2 collision cell.

Tandem instruments capable of MSN can be used with the disclosed method. As an example consider; a method where one selects a set of molecules using a first stage filter (MS), photocleaves these molecules to yield a set of multidimension signals, selects these multidimension signals using a second stage (MS/MS), alters these multidimension signals by collisional fragmentation, detects by time of flight (MS/MS/MS or MS3). Many other combinations are possible and the disclosed method can be adapted for use with such systems. For example, extension to more stages or analysis of multidimension signal fragments is within the skill of those in the art.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a multidimension signal is disclosed and discussed and a number of modifications that can be made to a number of molecules including the multidimension signal are discussed, each and every combination and permutation of multidimension signal and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Multidimension Signals

Multidimension signals (MDS) are special label components that can generate one or more predetermined patterns that serve to indicate whether a further level of analysis can or should be performed and/or which portion(s) of the analyzed material can or should be analyzed in a further level of analysis. Reporter signals and indicator signals are forms of multidimension signals. Multidimension signals are molecules that have at least one characteristic that allows the multidimension signals to be distinguished and/or separated from other multidimension signals or other sets of multidimension signals. Generally, multidimension signals need only be distinguishable and/or separable from other multidimension signals and/or sets of multidimension signals present in the same indicator level of analysis. Some multidimension signals, such as reporter signals, should also be distinguishable, following alteration of the reporter signals, from different reporter signals in a set of reporter signals in a reporter signal level of analysis. Thus, multidimension signals have two primary functions or features in the disclosed methods. Related differences that allow generation of a pattern in indicator levels of analysis and differences in altered forms of multidimension signals (generally reporter signals) that allow different multidimension signals to be distinguished in a reporter signal level of analysis.

As mentioned above, multidimension signals can be reporter signals and indicator signals. Reporter signals and indicator signals are thus two forms of multidimension signal. Useful forms of the disclosed methods can involve the use of at least one set of multidimension signals. Reporter signals, which are described in more detail below, are molecules that can be preferentially fragmented, decomposed, reacted, derivatized or otherwise modified or altered for detection. Indicator signals, which are described in more detail below, are molecules that have at least one characteristic that allows the indicator signal to be distinguished and/or separated from other multidimension signals. Generally, indicator signals need only be distinguishable and/or separable from other multidimension signals present in same level of analysis. Multidimension signals, reporter signals and indicator signals can be used in sets, both individually and together. Thus, for example, a set of reporter signals can be used with a set of indicator signals, two sets of reporter signals can be used together, and a set of reporter signals can be used with a single indicator signal.

The multidimension signals, such as reporter signals, can have two key features. First, the multidimension signals can be used in sets where all the multidimension signals in the set have similar properties. The similar properties allow the multidimension signals to be distinguished and/or separated from other molecules lacking one or more of the properties. In some embodiments, the multidimension signals in a set have the same mass-to-charge ratio (m/z). That is, the multidimension signals in a set are isobaric. This allows the multidimension signals to be separated precisely from other molecules based on mass-to-charge ratio. The result of the filtering is a huge increase in the signal to noise ratio (S/N) for the system, allowing more sensitive and accurate detection. The filtering can be used to produce predetermined patterns from the multidimension signals that indicate whether a second stage should be performed and/or which portion(s) of the analyzed material can or should be analyzed in the fragmentation stage.

Second, all the multidimension signals in a set can be fragmented, decomposed, reacted, derivatized, or otherwise modified to distinguish the different multidimension signals in the set. For example, the multidimension signals can be fragmented to yield fragments having the same or similar charge but different mass. This allows each multidimension signal in a set to be distinguished by the different mass-to-charge ratios of the fragments of the multidimension signals. This is possible since, although the unfragmented multidimension signals in a set are isobaric, the fragments of the different multidimension signals are not. Multidimension signals to be detected on the basis of mass-to-charge ratio and/or to be detected with the use of a mass spectrometer, can be referred to as mass spectrometer multidimension signals. Reporter signals are a form of multidimension signals that can have these features.

Differential distribution of mass in the fragments of the multidimension signals, such as reporter signals, can be accomplished in a number of ways. For example, multidimension signals of the same nominal structure (for example, peptides having the same amino acid sequence), can be made with different distributions of heavy isotopes, such as deuterium. All multidimension signals in the set would have the same number of a given heavy isotope, but the distribution of these would differ for different multidimension signals. Similarly, multidimension signals of the same general structure (for example, peptides having the same amino acid sequence), can be made with different distributions of modifications, such as methylation, phosphorylation, sulphation, and use of seleno-methionine for methionine. All multidimension signals in the set would have the same number of a given modification, but the distribution of these would differ for different multidimension signals. Multidimension signals of the same nominal composition (for example, made up of the same amino acids) can be made with different ordering of the subunits or components of the multidimension signal. All multidimension signals in the set would have the same number of subunits or components, but the distribution of these would be different for different multidimension signals. Multidimension signals having the same nominal composition (for example, made up of the same amino acids) can be made with a labile or scissile bond at a different location in the multidimension signal. All multidimension signals in the set would have the same number and order of subunits or components. Where the labile or scissile bond is present between particular subunits or components, the order of subunits or components in the multidimension signal can be the same except for the subunits or components creating the labile or scissile bond. Each of these modes can be combined with one or more of the other modes to produce differential distribution of mass in the fragments of the multidimension signals. For example, different distributions of heavy isotopes can be used in multidimension signals where a labile or scissile bond is placed in different locations. Further, each of these modes can be combined with each other, one or more of the other modes, and/or other multidimension signals to produce differential distribution of mass in the multidimension signals and sets of reporter signals, thus generating a pattern of masses that can be detected and used in an indicator level of analysis.

The multidimension signals, such as reporter signals and indicator signals, may be detected using mass spectrometry which allows sensitive distinctions between molecules based on their mass-to-charge ratios. The disclosed multidimension signals, such as reporter signals and indicator signals, can be used as general labels in myriad labeling and/or detection techniques. A set of isobaric multidimension signals can be used for multiplex labeling and/or detection of many analytes since the multidimension signal fragments can be designed to have a large range of masses, with each mass (or mass-to-charge ratio) individually distinguishable upon detection. A combination of isobaric and non-isobaric multidimension signals can allow patterns of mass (or mass-to-charge ratio) to be generated and can extend the multiplexing of the methods.

Thus, multidimension signals can be used in sets. For example, a set of multidimension signals that differ in some property or characteristic can be used to label different samples and/or analytes. In some forms of multidimension signals, the characteristic can be chosen to be compatible with a characteristic of reporter signals and/or other multidimension signals or sets of multidimension signals used in the same assay or assay system such that a recognizable pattern will result during analysis of the multidimension signals. For example, multidimension signals or sets of multidimension signals having masses (or mass-to-charge ratios) different from the mass (or mass-to-charge ratio) of other multidimension signals and sets of multidimension signals can be used in the same assay to generate characteristic patterns of mass (or mass-to-charge ratio) in mass spectrometry. Multidimension signals, reporter signals and indicator signals can be used in sets, both individually and together. Thus, for example, a set of reporter signals can be used with a set of indicator signals, two sets of reporter signals can be used together, and a set of reporter signals can be used with a single indicator signal.

The disclosed multidimension signals are preferably used in sets where members of a set have different mass-to-charge ratios (m/z) or in sets of sets where members of a set of multidimension signals have the same mass-to-charge ratio and the mass-to-charge ratios of members of different sets of the sets have different mass-to-charge ratios. This facilitates sensitive distinction of multidimension signals and/or sets of multidimension signals from each other and from other multidimension signals and/or sets of multidimension signals based on mass-to-charge ratio. Multidimension signals can have any structure that allows the generation of patterns with other multidimension signals in analysis of the disclosed methods.

Preferred multidimension signals (e.g., reporter signals or indicator signals) are made up of chains of subunits such as peptides, oligonucleotides, peptide nucleic acids, oligomers, carbohydrates, polymers, and other natural and synthetic polymers and any combination of these. Most preferred chains are peptides, and are referred to herein as multidimension signal peptides (or reporter signal peptides or indicator signal peptides, as the case may be). Chains of subunits and subunits have a relationship similar to that of a polymers and mers. The mers are connected together to form a polymer. Likewise, subunits are connected together to form chains of subunits. Preferred multidimension signals are made up of chains of similar or related subunits. These are termed homochains or homopolymers. For example, nucleic acids are made up of phosphonucleosides and peptides are made up of amino acids.

Multidimension signals can also be made up of heterochains or heteropolymers. A heterochain is a chain or a polymer where the subunits making up the chain are different types or the mers making up the polymer are different types. For example, a heterochain could be guanosine-alanine, which is made up of one nucleoside subunit and one amino acid subunit. It is understood that any combination of types of subunits can be used within the disclosed compositions, sets, and methods. Any molecule having the required properties can be used as a multidimension signal. Generally, multidimension signals need only be distinguishable and/or separable from other multidimension signals present in same level of analysis (such as an indicator level of analysis). Some multidimension signals, such as reporter signals, should also be distinguishable, following alteration of the reporter signals, from different reporter signals in a set of reporter signals in a reporter signal level of analysis.

Multidimension signals preferably are used in sets where all the indicator signals in the set have different physical properties and/or in sets of sets where the sets in a set of sets have different physical properties (the members of a given set in the set of sets can have the same physical properties). The different (or distinguishing) properties allow the multidimension signals and/or sets of multidimension signals to be distinguished and/or separated from other multidimension signals and/or sets of multidimension signals differing in one or more of the properties. As an example, the multidimension signals in a set have the same or different mass-to-charge ratios (m/z). That is, the multidimension signals in a set can be isobaric or non-isobaric. In general, within a set, indicator signals can be non-isobaric and reporter signals can be isobaric.

Multidimension signals can be used in combination with other multidimension signals. Generally, at least two different forms of multidimension signals can be used together in the same assay or assay system. The different forms of multidimension signals used together can generate one or more predetermined patterns during analysis which can then serve as an indicator that another level or dimension of analysis can be performed. Each level of analysis can, in turn, generate one or more predetermined patterns which can then serve as an indicator that another level or dimension of analysis can be performed. The disclosed method generally involves at least two levels of analysis, where the pattern generated in the first level of analysis indicates whether the second level of analysis should be performed. The pattern generated by analysis of multidimension signals can also be used to indicate which portion(s) of material being analyzed should be analyzed in the next level of analysis. Thus, for example, different portions or fractions of an analysis sample that is fractionated, separated or otherwise divided can be identified or selected for the next level of analysis based on detection of a predetermined pattern generated by the current level of analysis.

The pattern generated in an indicator level of analysis can be a result of one or more characteristics of the multidimension signals in the assay. For example, two or more different forms of multidimension signals can be used together in the same assay or assay system that differs in one or more characteristics. The different forms of multidimension signals used together can generate one or more predetermined patterns during analysis based on this difference in characteristics. For example, different forms of multidimension signals having characteristic differences in mass (or mass-to-charge ratio) can result in characteristic, predetermined patterns of mass (or mass-to-charge ratio) when analyzed by mass spectrometry. More specifically, if the members of one set of multidimension signals differ in mass (or mass-to-charge ratio) by a characteristic amount from the members of another set of multidimension signals, then members of the two sets of multidimension signals will generate mass spectrometry peaks that differ based on the characteristic mass (or mass-to-charge ratio) difference. This is true whether the multidimension signals are analyzed alone or if multidimension signal fusions or multidimension signal/analyte conjugates are analyzed because the same analyte fused or conjugated to the different forms of multidimension signals will generate mass spectrometry peaks that differ based on the characteristic mass (or mass-to-charge ratio) difference. The characteristic mass (or mass-to-charge ratio) difference can be, for example, the difference in mass (or mass-to-charge ratio) of the forms of multidimension signals, a multiple of the difference in mass (or mass-to-charge ratio) of the forms of multidimension signals, or a combination of the difference in mass (or mass-to-charge ratio) of the forms of multidimension signals and the total mass of the multidimension signals.

For use in a given indicator level of analysis, it is useful that the multidimension signals and sets of multidimension signals used have properties that are related or closely spaced. For example, multidimension signals and sets of multidimension signals having different mass-to-charge ratios (that generates a pattern of masses) can have relatively small differences in mass-to-charge ratio. This allows the multidimension signals (and/or the proteins or other analytes to which they are attached) to be separated precisely from other molecules based on the properties (such as mass-to-charge ratio) and to generate a pattern (such as a pattern of masses) with each other and with other multidimension signals. This also allows the predetermined pattern to be more easily identified.

It is preferred that the common property of multidimensional signals (e.g., reporter signals or indicator signals), multidimension signal/analyte conjugates, fragment conjugates, multidimension signal fusions, multidimension signal fusion fragments, or multidimension signal peptides, or the property of a multimension signal (e.g., a reporter signal or indicator signal) to form a pattern is not an affinity tag. Nevertheless, even in such a case, multidimensional signals, multidimension signal/analyte conjugates, fragment conjugates, multidimension signal fusions, multidimension signal fusion fragments, or multidimension signal peptides that otherwise have a common property may also include an affinity tag—and in fact may all share the same affinity tag—so long as another common property is present that can be (and, in some embodiments of the disclosed method, is) used to separate multidimensional signals, multidimension signal/analyte conjugates, fragment conjugates, multidimension signal fusions, multidimension signal fusion fragments, or multidimension signal peptides sharing the common property from other molecules lacking the common property or so long as another property is present that can be (and, in some embodiments of the disclosed method, is) used to generate a pattern. With this in mind, it is preferred that, if chromatography or other separation techniques are used to separate multidimensional signals, multidimension signal/analyte conjugates, fragment conjugates, multidimension signal fusions, multidimension signal fusion fragments, or multidimension signal peptides based on the common property, the affinity be based on an overall physical property of the reporter signals and not on the presence of, for example, a feature or moiety such as an affinity tag. As used herein, a common property is a property shared by a set of components (such as multidimension signals, multidimension signal/analyte conjugates, fragment conjugates, multidimension signal fusions, multidimension signal fusion fragments, or multidimension signal peptides). That is, the components have the property “in common.” It should be understood that multidimensional signals, multidimension signal/analyte conjugates, fragment conjugates, multidimension signal fusions, multidimension signal fusion fragments, or multidimension signal peptides in a set may have numerous properties in common. However, as used herein, the common properties of multidimensional signals, multidimension signal/analyte conjugates, fragment conjugates, multidimension signal fusions, multidimension signal fusion fragments, or multidimension signal peptides referred to are only those used in the disclosed method to distinguish and/or separate the multidimensional signals, multidimension signal/analyte conjugates, fragment conjugates, multidimension signal fusions, multidimension signal fusion fragments, or multidimension signal peptides sharing the common property from molecules that lack the common property. Further, as used herein, the properties of the multidimension signals (e.g., reporter signals and indicator signals) used to generate a patern (“pattern-generating properties”) are only those used in the disclosed methods to generate the pattern.

Predetermined patterns can include any features, characteristics, properties or the like of the multidimension signals. Patterns generally involve differences in the features, characteristics, properties or the like; and in particular, patterns can involve, for example, specific, repeatable, characteristic, expected or consistent differences in the features, characteristics, properties or the like. For example, a pattern can be specific differences in mass-to-charge ratio among two or more multidimension signals. In general, patterns involve two or more different identities or values of the features, characteristics, properties or the like. That is, a pattern generally involves a difference between the identity or value of a feature, characteristic, property or the like of different multidimension signals.

Predetermined patterns in features, characteristics, properties or the like of multidimension signals can be formed from any useful or desired combination of identities or values of the features, characteristics, properties or the like. For example, two, two or more, three, three or more, four, four or more, five, five or more, six, six or more, seven, seven or more, eight, eight or more, nine, nine or more, ten, ten or more, eleven, eleven or more, twelve, twelve or more, thirteen, thirteen or more, fourteen, fourteen or more, fifteen, fifteen or more, sixteen, sixteen or more, seventeen, seventeen or more, eighteen, eighteen or more, nineteen, nineteen or more, twenty, twenty or more, 21, 21 or more, 22, 22 or more, 23, 23 or more, 24, 24 or more, 25, 25 or more, 26, 26 or more, 27, 27 or more, 28, 28 or more, 29, 29 or more, 30, 30 or more, 35, 35 or more, 40, 40 or more, 45, 45 or more, 50, 50 or more, 55, 55 or more, 60, 60 or more, 65, 65 or more, 70, 70 or more, 75, 75 or more, 80, 80 or more, 85, 85 or more, 90, 90 or more, 95, 95 or more, 100, 100 or more, or any combination of these numbers of identities or values of the features, characteristics, properties or the like can be used as the predetermined pattern.

A variety of different properties can be used as the physical property used to generate a pattern from multidimension signals, a pattern for indicator level of analysis, or a pattern to separate multidimension signals (e.g., reporter signals or indicator signals) or to separate multidimension signal/analyte conjugates, fragment conjugates, multidimension signal fusion, multidimension signal fusion fragments and/or multidimension signal peptides from other molecules lacking the common property. For example, non-limiting physical properties useful as a pattern of a common property include mass, charge, isoelectric point, hydrophobicity, chromatography characteristics, and density. In one embodiment, the physical property used to generate a pattern or the physical property shared by multidimension signal/analyte conjugates, fragment conjugates, multidimension signal fusion, multidimension signal fusion fragments or multidimension signal peptides in a set (and used to distinguish or separate the multidimension signal/analyte conjugates fragment conjugates, multidimension signal fusion, multidimension signal fusion fragments or multidimension signal peptides) is an overall property of the multidimension signals, multidimension signal/analyte conjugates, fragment conjugate, multidimension signals fusion, multidimension signal fusion fragments and/or multidimension signal peptides (for example, overall mass, overall charge, isoelectric point, overall hydrophobicity, etc.) rather than the mere presence of a feature or moiety (for example, an affinity tag, such as biotin). Such properties are referred to herein as “overall” properties (and thus, multidimension signal/analyte conjugates, fragment conjugates, multidimension signal fusion, multidimension signal fusion fragments or multidimension signal peptides in a set would be referred to as sharing a “common overall property”). It should be understood that multidimension signals (e.g., reporter signals or indicator signals), multidimension signal/analyte conjugates, fragment conjugates, multidimension signal fusion, multidimension signal fusion fragments and/or multidimension signal peptides can have features and moieties, such as affinity tags, and that such features and moieties can contribute to the overall property (by contributing mass, for example). However, such limited and isolated features and moieties generally would not serve as the sole basis of the overall property.

Sets of multidimension signals (e.g., reporter signals and indicator signals) can have any number of multidimension signals. For example, sets of multidimension signals can have one, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, three hundred or more, four hundred or more, or five hundred or more different multidimension signals. Although specific numbers of multidimension signals and specific endpoints for ranges of the number of multidimension signals are recited, each and every specific number of multidimension signals and each and every specific endpoint of ranges of numbers of multidimension signals are specifically contemplated, although not explicitly listed, and each and every specific number of multidimension signals and each and every specific endpoint of ranges of numbers of multidimension signals are hereby specifically described.

The sets of multidimension signals can be made up of multidimension signals that are made up of chains or polymers. The set of multidimension signals can be homosets which means that the set is made up of one type of multidimension signal or that the multidimension signal is made up of homochains or homopolymers. The set of multidimension signals can also be a heteroset which means that the set is made up of different multidimension signals or of multidimension signals that are made up of different types of chains or polymers. A special type of heteroset is one in which the set is made up of different homochains or homopolymers, for example one peptide chain and one nucleic acid chain. Another special type of heteroset is one where the chains themselves are heterochains or heteropolymers. Still another type of heteroset is one which is made up of both heterochains/heteropolymers and homochains/homopolymers.

The disclosed multidimension signals can be associated with, incorporated into, or otherwise linked to analytes or proteins. Multidimension signal can also be in conjunction with analytes or proteins (such as in mixtures of multidimension signals and analytes or proteins), where no significant physical association between the multidimension signals and analytes or between the multidimension signals and proteins occurs; or alone, where no analyte or protein is present.

In cases where multidimension signals are not or are no longer associated with analytes or proteins, sets of multidimension signals can be used where two or more of the multidimension signals in a set have one or more properties that generate a pattern in an indicator level of analysis. Further, where reporter signals are not or are no longer associated with analytes, sets of reporter signals can be used where two or more of the reporter signals in a set have one or more common properties that allow the reporter signals having the common property to be distinguished and/or separated from other molecules lacking the common property. Detection of the multidimension signals indicates the presence of the corresponding analytes or proteins.

The multidimension signals are preferably detected using mass spectrometry which allows sensitive distinctions between molecules based on their mass-to-charge ratios. The disclosed multidimension signals can be used as general labels in myriad labeling and/or detection techniques.

Some forms of multidimension signals (e.g., reporter signals or indicator signals) can include one or more affinity tags. Such affinity tags can allow the detection, separation, sorting, or other manipulation of the labeled proteins, labeled analytes, multidimension signals, multidimension signal fragments, or multidimension signal fusions based on the affinity tag. For indicator signals, such affinity tags are separate from and in addition to (not the basis of) the properties of a set of indicator signals used to generate a pattern. Rather, such affinity tags serve the different purpose of allowing manipulation of a sample prior to or as a part of the disclosed method, not the means to separate indicator signals based on the pattern-generating property. For reporter signals, such affinity tags are separate from and in addition to (not the basis of) the common properties of a set of reporter signals that allows separation of reporter signals from other molecules. Rather, such affinity tags serve the different purpose of allowing manipulation of a sample prior to or as a part of the disclosed method, not the means to separate reporter signals based on the common property.

Multidimension signals (e.g., reporter signals or indicator signals) can have none, one, or more than one affinity tag. Where a multidimension signal has multiple affinity tags, the tags on a given multidimension signal can all be the same or can be a combination of different affinity tags. Following the principles described above and elsewhere herein, affinity tags also can be used to change mass and/or charge differentially on indicator signals, and can be used to distribute mass and/or charge differentially on reporter tags. Affinity tags can be used with multidimension signals in a manner similar to the use of affinity labels as described in PCT Application WO 00/11208.

Peptide-DNA conjugates (Olejnik et al., Nucleic Acids Res., 27(23):4626-31 (1999)), synthesis of PNA-DNA constructs, and special nucleotides such as the photocleavable universal nucleotides of WO 00/04036 can be used as indicator signals in the disclosed method. Useful photocleavable linkages are also described by Marriott and Ottl, Synthesis and applications of heterobifunctional photocleavable cross-linking reagents, Methods Enzymol. 291:155-75 (1998).

Photocleavable bonds and linkages are useful in (and for use with) multidimension signals because it allows precise and controlled release of multidimension signals from analytes or proteins (or other intermediary molecules) to which they are attached. A variety of photocleavable bonds and linkages are known and can be adapted for use in and with indicator signals. Recently, photocleavable amino acids have become commercially available. For example, an Fmoc protected photocleavable slightly modified phenylalanine (Fmoc-D,L-β Phe(2-NO₂)) is available (Catalog Number 0011-F; Innovachem, Tucson, Ariz.). The introduction of the nitro group into the phenylalanine ring causes the amino acid to fragment under exposure to UV light (at a wavelength of approximately 350 nm). The nitrogen laser emits light at approximately 337 nm and can be used for fragmentation. The wavelength used will not cause significant damage to the rest of the peptide.

Fmoc synthesis is a common technique for peptide synthesis and Fmoc-derivative photocleavable amino acids can be incorporated into peptides using this technique. Although photocleavable amino acids are usable in and with any multidimension signal, they are particularly useful in peptide multidimension signals (e.g., peptide reporter signals and peptide indicator signals).

Use of photocleavable bonds and linkages in and with multidimension signals can be illustrated with the following examples. Materials on a blank plastic substrate (for example, a Compact Disk (CD)) may be directly measured from that surface using a MALDI source ion trap. For example, a thin section of tissue sample, flash frozen, could be applied to the CD surface. A multidimension signal molecule (for example, an antibody with a multidimension signal attached via a photocleavable linkage) can be applied to the tissue surface. Recognition of specific components within the tissue allows for some of the antibody/multidimension signal conjugates to associate (excess conjugate is removed during subsequent wash steps). The multidimension signal then can be released from the antibody by applying a UV light and detected directly using the MALDI ion trap instrument.

For example, a peptide of sequence CF*XXXXXDPXXXXXR (SEQ ID NO:9) (which contains a reporter signal) can be attached to an antibody using a disulfide bond linkage method. Exposure to the UV source of a MALDI laser will cleave the peptide at the modified phenylalanine, F*, releasing the XXXXXDPXXXXXR reporter signal (amino acids 3-15 of SEQ ID NO:9). The reporter signal subsequently can be fragmented at the DP bond and the charged fragment detected as described elsewhere herein. In another example, a peptide of sequence CF*XXXXXXXXXXXXR (SEQ ID NO:12) (which contains a indicator signal) can be attached to an antibody using a disulfide bond linkage method. Exposure to the UV source of a MALDI laser will cleave the peptide at the modified phenylalanine, F*, releasing the XXXXXXXXXXXXR indicator signal (amino acids 3-15 of SEQ ID NO:12).

Another example of the use of photocleavable linkages with multidimension signals involves DNA-peptide chimeras used as multidimension signal molecules. Such multidimension signal molecules are useful as probes to detect particular nucleic acid sequences. In a DNA-peptide chimera (or PNA-peptide chimera), the peptide portion can be or include a multidimension signal. Placement of a photocleavable phenylalanine, for example, near the DNA peptide junction of the multidimension signal molecule allows for the release of the multidimension signal from the multidimension signal molecule by UV light. The released multidimension signal can be detected directly or fragmented and detected as described elsewhere herein. Similarly to the case of the antibody-peptide multidimension signal molecule described above, the DNA-peptide chimera can be associated with a nucleic acid molecule present on the surface of a substrate such as a CD and the multidimension signal released using the UV source of a MALDI laser.

Multiple photocleavable bonds and/or linkages can be used in or with the same multidimension signals or multidimension signal conjugates (such as multidimension signal molecules or multidimension signal fusions) to achieve a variety of effects. For example, different photocleavable linkages that are cleaved by different wavelengths of light can be used in different parts of multidimension signals or multidimension signal conjugates to be cleaved at different stages of the method. Different fragmentation wavelengths allow sequential processing which enables, for example, the combinations of the release and fragmentation methods.

As an example, a peptide containing two photocleavable amino acids, Z (cleavage wavelength in the infrared) and F* (photocleavable phenylalanine, cleavage wavelength in UV) can be constructed of the form XZXXXXXXF*XXXXXXR where the amino terminus can be attached to an analyte or other molecule utilizing known chemistry. The result is a reporter signal/analyte conjugate (or, alternatively, a reporter molecule), or an indicator signal/analyte conjugate (or, alternatively, an indicator molecule). The multidimension signal can be released from the conjugate by exposing the conjugate to an appropriate wavelength of light (infrared in this example), thus cleaving the bond at Z. Once the parent ion is selected and stored in the ion trap, the multidimension signal can be fragmented by exposing it to an appropriate wavelength of light (UV in this example) to produce the daughter ion (XXXXXXR⁺) which can be detected and quantitated.

Other labels that can be used as multidimension signals, reporter signals and/or indicator signals are described in U.S. Application Nos. 2004/0018565, 2003/0100018, 2003/0050453, 2004/0023274, 2002/014673, 2003/0022225, and U.S. Pat. Nos. 6,312,893, 6,312,904, 6,629,040, and Geysen et al. (Chemistry & Biology 3(8):679-688 (1996)), all of which are incorporated by reference herein.

Multidimension signals can be attached, coupled or immobilized to any desired analyte, compound, substrate, or other composition using any suitable technique. As used herein, molecules are coupled when they are covalently joined, directly or indirectly. One form of indirect coupling is via a linker molecule. The multidimension signal can be coupled to the analyte, compound, substrate, or other composition by any suitable coupling reactions. Many chemistries and techniques for coupling compounds are known and can be used to couple multidimension signals to analytes. For example, coupling can be made using thiols, epoxides, nitriles for thiols, NHS esters, isothiocyantes, isothiocyanates for amines, amines, and alcohols for carboxylic acids. As another example, peptide multidimension signals can be coupled via acetylation of primary amines is known (Wetzel et al., Bioconjugate Chem 1, 114-122 (1990)).

B. Reporter Signals

Reporter signals (also called reporter signal peptides) are molecules that can be preferentially fragmented, decomposed, reacted, derivatized, or otherwise modified or altered for detection. Reporter signals are a form of multidimension signal.

Reference to multidimension signals and their derivatives having the properties of reporter signals and their derivatives can be considered the same as a reporter signal version of the multidimension signal (and the labels can be interchanged in such circumstances). Detection of the modified reporter signals is preferably accomplished with mass spectrometry. The disclosed reporter signals are preferably used in sets where members of a set have the same mass-to-charge ratio (m/z). This facilitates sensitive filtering or separation of reporter signals from other molecules based on mass-to-charge ratio. Reporter signals can have any structure that allows modification of the reporter signal and identification of the different modified reporter signals. Reporter signals preferably are composed such that at least one preferential bond rupture can be induced in the molecule. A set of reporter signals having nominally the same molecular mass and arbitrarily chosen internal fragmentation points may be constructed such that upon fragmentation each member of the set will yield unique correlated daughter fragments. For convenience, reporter signals that are fragmented, decomposed, reacted, derivatized, or otherwise modified for detection are referred to as fragmented reporter signals. Preferred reporter signals can be fragmented in tandem mass spectrometry.

Reporter signals preferably are used in sets where all the reporter signals in the set have similar physical properties. The similar (or common) properties allow the reporter signals to be distinguished and/or separated from other molecules lacking one or more of the properties. Preferably, the reporter signals in a set have the same mass-to-charge ratio (m/z). That is, the reporter signals in a set can be isobaric. This allows the reporter signals (and/or the proteins or other analytes to which they are attached) to be separated precisely from other molecules based on mass-to-charge ratio. The result of the filtering is a huge increase in the signal to noise ratio (S/N) for the system, allowing more sensitive and accurate detection. Sets of reporter signals can have any number of reporter signals.

A preferred common overall property is the property of subunit isomers. This property occurs when a set of at least two reporter signals (which typically are made up of subunit chains which are in turn made up of subunits, for example, like the relationship between a polymer and the units that make up a polymer) is made up of subunit isomers, and the set could then be called subunit isomeric or isomeric for subunits. Subunits are discussed elsewhere herein, but reporter signals can be made up of any type of chain, such as peptides or nucleic acids or polymer (general) which are in turn made up of subunits for example amino acids and phosphonucleosides, and mers (general) respectively. Within each type of subunit there are typically multiple members that are all the same type of subunit, but differ. For example, within the subunit type “amino acids,” there are many members, for example, ala, tyr, and ser, or any other combination of amino acids.

When a set of reporter signals is subunit isomeric or is made up of subunit isomers this means that each individual of the set is a subunit isomer of every other individual subunit in the set. Isomer or isomeric means that the makeup of the subunits forming the subunit chain (i.e., distribution or array) is the same but the overall connectivity of the subunits, forming the chain, is different. Thus, for example, a first reporter signal could be the chain, ala-ser-lys-gln, a second reporter signal could be the chain ala-lys-ser-gln, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, i.e. each has one ala, one ser, one lys, and one gin, but each chain has a different connectivity. If, however, the set of reporter signals were made which contained the first, second, and third reporter signals the set would not be isomeric because the make up of each chain would not be the same because the first and second chains do not have a pro and the third chain does not have a gln.

Another illustration is the following: a first reporter signal could be the chain, ala-guanosine-lys-adenosine, a second reporter signal could be the chain ala-adenosine-lys-guanosine, and a third reporter signal could be the chain ala-ser-lys-pro. If a set of reporter signals was made that contained the first reporter signal and the second reporter signal, the set would be subunit isomeric because the first reporter signal and the second reporter signal have the same makeup, i.e. each has one ala, one guanosine, one lys, and one adenosine, but each chain has a different connectivity. If, however, the set of reporter signals were made which contained the first, second, and third reporter signals the set would not be isomeric because the makeup of each chain would not be the same because the first and second chains do not have a pro or a ser and the third chain does not have a guanosine or adenosine. This illustration shows that the sets can be made up of, or include, heterochains and still be considered subunit isomers.

Reporter signals in a set can be fragmented, decomposed, reacted, derivatized, or otherwise modified or altered to distinguish the different reporter signals in the set. Preferably, the reporter signals are fragmented to yield fragments of similar charge but different mass. The reporter signals can also be fragmented to yield fragments of different charge and mass. Such changes allow each reporter signal in a set to be distinguished by the different mass-to-charge ratios of the fragments of the reporter signals. This is possible since, although the unfragmented reporter signals in a set can be isobaric, the fragments of the different reporter signals are not. Thus, a key feature of the disclosed reporter signals is that the reporter signals have a similarity of properties while the modified reporter signals are distinguishable.

Differential distribution of mass in the fragments of the reporter signals can be accomplished in a number of ways. For example, reporter signals of the same nominal structure (for example, peptides having the same amino acid sequence), can be made with different distributions of heavy isotopes, such as deuterium (²H), tritium (³H) ¹⁷O, ¹⁸O, ¹³C, or ¹⁴C; stable isotopes are preferred. All reporter signals in the set would have the same number of a given heavy isotope, but the distribution of these would differ for different reporter signals. An example of such a set of reporter signals is A*G*SLDPAGSLR, A*GSLDPAG*SLR, and AGSLDPA*G*SLR (SEQ ID NO:2), where the asterisk indicates at least one heavy isotope substituted amino acid. For a singly charged parent ion and, following fragmentation at the scissile DP bond, one predominantly charged daughter, there are three distinguishable primary daughter ions, PAGSLR⁺, PAG*SLR⁺, PA*G*SLR⁺ (amino acids 6-11 of SEQ ID NO:2).

Similarly, reporter signals of the same general structure (for example, peptides having the same amino acid sequence), can be made with different distributions of modifications or substituent groups, such as methylation, phosphorylation, sulphation, and use of seleno-methionine for methionine. All reporter signals in the set would have the same number of a given modification, but the distribution of these would differ for different reporter signals. An example of such a set of reporter signals is AGS*M*LDPAGSMLR, AGS*MLDPAGSM*LR, and AGS*MLDPAGS*M*LR (SEQ ID NO:3), where S* indicates phosphoserine rather than serine, and, M* indicates seleno-methionine rather than methionine. For a singly charged parent ion and, following fragmentation at the scissile DP bond, one predominantly charged daughter, there are three distinguishable primary daughter ions, PAGSMLR⁺, PAGSM*LR⁺, PAGS*M*LR⁺ (amino acids 7-13 of SEQ ID NO:3).

Reporter signals of the same nominal composition (for example, made up of the same amino acids), can be made with different ordering of the subunits or components of the reporter signal. All reporter signals in the set would have the same number of subunits or components, but the distribution of these would be different for different reporter signals. An example of such a set of reporter signals is AGSLADPGSLR (SEQ ID NO:4), ALSLADPGSGR (SEQ ID NO:5), ALSLGDPASGR (SEQ ID NO:6). For a singly charged parent ion and, following fragmentation at the scissile DP bond, one predominantly charged daughter, there are three distinguishable primary daughter ions, PGSLR⁺ (amino acids 7-11 of SEQ ID NO:4), PGSGR⁺ (amino acids 7-11 of SEQ ID NO:5), PASGR⁺ (amino acids 7-11 of SEQ ID NO:6).

Reporter signals having the same nominal composition (for example, made up of the same amino acids), can be made with a labile or scissile bond at a different location in the reporter signal. All reporter signals in the set would have the same number and order of subunits or components. Where the labile or scissile bond is present between particular subunits or components, the order of subunits or components in the reporter signal can be the same except for the subunits or components creating the labile or scissile bond. Reporter signal peptides used in reporter signal fusions preferably use this form of differential mass distribution. An example of such a set of reporter signals is AGSLADPGSLR (SEQ ID NO:4), AGSDPLAGSLR (SEQ ID NO:7), ADPGSLAGSLR (SEQ ID NO:8). For a singly charged parent ion and, following fragmentation at the scissile DP bond, one predominantly charged daughter, there are three distinguishable primary daughter ions, PGSLR⁺(amino acids 7-11 of SEQ ID NO:4), PLAGSLR⁺ (amino acids 5-11 of SEQ ID NO:7), PGSLAGSLR⁺ (amino acids 3-11 of SEQ ID NO:8).

Each of these modes can be combined with one or more of the other modes to produce differential distribution of mass in the fragments of the reporter signals. For example, different distributions of heavy isotopes can be used in reporter signals where a labile or scissile bond is placed in different locations. Different mass distribution can be accomplished in other ways. For example, reporter signals can have a variety of modifications introduced at different positions. Some examples of useful modifications include acetylation, methylation, phosphorylation, seleno-methionine rather than methionine, sulphation. Similar principles can be used to distribute charge differentially in reporter signals. Differential distribution of mass and charge can be used together in sets of reporter signals.

Reporter signals can also contain combinations of scissile bonds and labile bonds. This allows more combinations of distinguishable signals or to facilitate detection. For example, labile bonds may be used to release the isobaric fragments, and the scissile bonds used to decode the proteins.

Selenium substitution can be used to alter the mass of reporter signals. Selenium can substitute for sulfur in methionine, resulting in the modified amino acid selenomethionine. Selenium is approximately forty seven mass units larger than sulfur. Mass spectrometry may be used to identify peptides or proteins incorporating selenomethionine and methionine at a particular ratio. Small proteins and peptides with known selenium/sulfur ratio are preferably produced by chemical synthesis incorporating selenomethionine and methionine at the desired ratio. Larger proteins or peptides may be by produced from an E. coli expression system, or any other expression system that inserts selenomethionine and methionine at the desired ratio (Hendrickson et al., Selenomethionyl proteins producedfor analysis by multiwavelength anomalous diffraction (MAD): a vehicle for direct determination of three-dimensional structure. Embo J, 9(5):1665-72 (1990), Cowie and Cohen, Biosynthesis by Escherichia coli of active altered proteins containing selenium instead of sulfur. Biochimica et Biophysica Acta, 26:252-261 (1957), and Oikawa et al., Metalloselenonein, the selenium analogue of metallothionein: synthesis and characterization of its complex with copper ions. Proc Natl Acad Sci USA, 88(8):3057-9 (1991).

As mentioned above, a reporter signal can include a photocleavable linkage to allows precise and controlled release of reporter signals from analytes or proteins (or other intermediary molecules) to which they are attached. A photocleavable linkage also can be incorporated into a reporter signal and used for fragmentation of the reporter signal in the disclosed methods. For example, a photocleavable amino acid (such as the photocleavable phenylalanine) can be incorporated at any desired position in a peptide reporter signal. A reporter signal such as XXXXXXF*XXXXXR containing photocleavable phenylalanine (F*) that is photocleavable. The reporter signal can then be fragmented using the appropriate wavelength of light and the charged fragment detected. When ionizing the reporter signal (from a surface, for example) for detection, a MALDI laser that does not cause significant photocleavage (for example, Er:YAG at 2.94 μm) can be used for ionization and a second laser (for example, Nitrogen at 337 nm) can be used to fragment the reporter signal. In this case XXXXXXFXXXXXR⁺ would be photocleaved to yield XXXXXR⁺. The second laser may intersect the reporter signal ion packet at any location. Modification to the vacuum system of a mass spectrometer for this purpose is straightforward.

The use of photocleavable linkages in reporter signals is particularly useful when the analyte or protein (or other component) to which the reporter signal is attached could fragment at a scissile bond in a collision cell. For example, in reporter signal fusions, a protein fragment/reporter signal polypeptide could be generated that contained a scissile bond in both the protein fragment portion and the reporter signal portion. An example would be XXXXXXXXXDPXXX(XXXXXXXDPXXXXXXXR)XXXX (SEQ ID NO: 10), where the sequence in parenthesis indicate the reporter signal portion and the DP dipeptides contain scissile bonds and where X is any amino acid. Fragmenting this polypeptide in a collision cell could result in fragmentation at either or both of the DP bonds, thus complicating the fragment spectrum. Use of a photocleavable linkage (such as a photocleavable amino acid) in the reporter signal portion would allow specific photocleavage of the reporter signal during analysis. For example, an analogous polypeptide XXXXXXXXXDPXXX(XXXXXXXF*XXXXXXXR)XXXX (SEQ ID NO:11) would allow specific photocleavage a the F* position of the reporter signal.

Reporter signal calibrators are a special form of reporter signal characterized by their use in reporter signal calibration. Reporter signal calibrators can be any form of reporter signal, as described above and elsewhere herein, but are used as separate molecules that are not physically associated with analytes or proteins being assessed. Thus, reporter signal calibrators need not (and preferably do not) have reactive groups for coupling to analytes or proteins and need not be (and preferably are not) associated with specific binding molecules or other molecules or components described herein as being associated with reporter signals.

Reporter signal calibrators preferably share one or more common properties with one or more analytes. Reporter signal calibrators and analytes that share one or more common properties are referred to as a reporter signal calibrator/analyte set. When only one analyte and one reporter signal calibrator share the common property they also can be referred to as a reporter signal calibrator/analyte pair. Reporter signal calibrators and analytes in a reporter signal calibrator/analyte set are said to be matching. The common property allows a reporter signal calibrator and its matching analyte to be distinguished and/or separated from other molecules lacking one or more of the properties. Preferably, the reporter signal calibrators and analytes in a set have the same mass-to-charge ratio (m/z). That is, the matching reporter signal calibrators and analytes in a set can be isobaric. This allows the reporter signal calibrators and analytes to be separated precisely from other molecules based on mass-to-charge ratio. Reporter signal calibrators can be fragmented, decomposed, reacted, derivatized, or otherwise modified or altered to distinguish the altered reporter signal calibrators from their matching analytes. The analytes can also be fragmented. Rhe reporter signal calibrators are fragmented to yield fragments of similar charge but different mass, or can be fragmented to yield fragments of different charge and mass. Such changes allow the reporter signal calibrator to be distinguished from its matching analyte (and other analytes and/or reporter signal calibrators that are members of the same set, if any) by the different mass-to-charge ratio of the fragment of the reporter signal calibrator. This is possible since, although the unfragmented reporter signal calibrator(s) and analyte(s) in a set are isobaric, the fragments of the reporter signal calibrator(s) are not. Thus, a key feature of the disclosed reporter signal calibrators is that the reporter signal calibrators have a similarity of properties with their matching analytes while the modified reporter signal calibrators are distinguishable from their matching analytes.

Preferred analytes for use with reporter signal calibrators are proteins, peptides, and/or protein fragments (collectively referred to for convenience as proteins). Reporter signal calibrators and proteins that share one or more common properties are referred to as a reporter signal calibrator/protein set. When only one protein and one reporter signal calibrator share the common property they also can be referred to as a reporter signal calibrator/protein pair. Reporter signal calibrators and proteins in a reporter signal calibrator/analyte set are said to be matching.

As described elsewhere herein, reporter signal calibrators can be used as standards for assessing the presence and amount of analytes in samples. For this purpose, a reporter signal calibrator designed for each analyte to be assessed can be mixed with the sample to be analyzed. Analytes and their matching reporter signal calibrators are then processed together to result in detection of both analytes and reporter signal calibrators (preferably in their altered forms). The amount of reporter signal calibrator or altered reporter signal calibrator detected provides a standard (since the amount of reporter signal calibrator added can be known) against which the amount of analyte or altered analyte detected can be compared. This allows the amount of analyte present in the sample to be accurately gauged.

i-PROT labels can be used as multidimension signals and reporter signals in the disclosed compositions and methods. i-PROT systems and labels, referred to as reporter signals, are described in U.S. Application No. 2003/0194717, U.S. Application No. 2004/0220412, U.S. Application No. 2003/0124595, and U.S. Pat. No. 6,824,981, all of which are incorporated by reference herein for their descriptions of reporter signals and use of reporter signals for labeling and detecting. In the i-PROT system, reporter signals can be attached to analytes such as proteins in any manner.

In i-PROT systems, the reporter signals preferably are fragmented to yield fragments of similar charge but different mass. This allows each labeled analyte (and/or each reporter signal) in a set to be distinguished by the different mass-to-charge ratios of the fragments of the reporter signals. This is possible since, although the unfragmented reporter signals in a set are isobaric, the fragments of the different reporter signals are not. In i-PROT systems, reporter signals can be used in sets where all the reporter signals in the set have similar properties (such as similar mass-to-charge ratios). The similar properties allow the reporter signals to be distinguished and/or separated from other molecules lacking one or more of the properties. Preferably, the reporter signals in a set have the same mass-to-charge ratio (m/z). That is, the reporter signals in a set are isobaric.

iTRAQ labels can be used as multidimension signals and reporter signals in the disclosed compositions and methods. iTRAQ systems and labels are described in U.S. Application No. 2004/0220412, and in PCT Application No. WO2004/070352, both of which are incorporated by reference herein for their descriptions of iTRAQ labels and use of iTRAQ labels for labeling and detecting. iTRAQ is a labeling system using a multiplexed set of reagents for quantitative protein analysis that places isobaric mass labels at the N-termini and lysine side chains of peptides in a digest mixture. The reagents are differentially isotopically labeled such that all derivatized peptides are isobaric and chromatographically indistinguishable, but yield signature or reporter ions following CID that can be used to identify and quantify individual members of the multiplex set. Thus, iTRAQ labels are a form of reporter signals. iTRAQ labels are amine-specific, stable isotope reagents that can label all peptides in up to four different biological samples simultaneously, enabling relative and absolute quantitation from MS/MS spectra. In the iTRAQ system, the reporter can be a 5, 6 or 7 membered heterocyclic ring comprising a ring nitrogen atom that is N-alkylated with a substituted or unsubstituted acetic acid moiety to which the analyte is linked through the carbonyl carbon of the N-alkyl acetic acid moiety, wherein each different label comprises one or more heavy atom isotopes. The heterocyclic ring can be substituted or unsubstituted. The heterocyclic ring can be aliphatic or aromatic. Possible substituents of the heterocylic moiety include alkyl, alkoxy and aryl groups. The substituents can comprise protected or unprotected groups, such as amine, hydroxyl or thiol groups, suitable for linking the analyte to a support. The heterocyclic ring can comprise additional heteroatoms such as one or more nitrogen, oxygen or sulfur atoms.

The components of an example of the multiplexed derivatization chemistry of iTRAQ labeling are shown in FIGS. 6 and 7. As described in Ross et al., MCP Paper in Press, Manuscript M400129-MCP200 (Sep. 28, 2004), a reduced and alkylated digest mixture of 6 proteins was split into 4 identical aliquots. Ross et al. is incorporated by reference herein for its descriptions of iTRAQ labels and use of iTRAQ labels for labeling and detecting. Each was then labeled with one of the four isotopically labeled tags, and the derivatized digests combined in mixtures of varying proportions. The multiplex isobaric tags produce abundant MS/MS signature ions at m/z 114.1, 115.1, 116.1, 117.1 and the relative areas of these peaks correspond with the proportions of the labeled peptides.

The mass shift imposed by isotopic enrichment of each signature ion in this example of iTRAQ is balanced with isotopic enrichment at the carbonyl component of the derivative, such that the total mass of each of the four tags is identical. Thus any given peptide labeled with each of the four tags has the same nominal mass, which provides a sensitivity enhancement over mass-difference labeling. With isobaric peptides, the MS ion current at a given peptide mass is the sum of ion current from all samples in the mixture, so there is no splitting of MS precursor signal and no increase in spectral complexity by combining two or more samples (FIG. 6, FIG. 7). The sensitivity enhancement is carried over into MS/MS spectra, since all of the peptide backbone fragments ions are also isobaric (FIG. 7).

In FIG. 6, diagrams of the structure of iTRAQ multiplexed isobaric tagging chemistry are shown. FIG. 6A shows the complete molecule consists of a reporter group (based on N-methylpiperazine) a mass balance group (carbonyl) and a peptide reactive group (NHS ester). The overall mass of reporter and balance components of the molecule are kept constant using differential isotopic enrichment with ¹³C and 180 atoms, thus avoiding problems with chromatographic separation seen with enrichment involving deuterium substitution. The reporter group ranges in mass from m/z 114.1 to 117.1, while the balance group ranges in mass from 28 to 31 Da, such that the combined mass remains constant (145.1 Da) for each of the 4 reagents. FIG. 6B shows the structure when the tag is reacted with a peptide and forms an amide linkage to a peptide amine (N-terminal or epsilon amino group of lysine). These amide linkages fragment in a similar fashion to backbone peptide bonds when subjected to collision induced dissociation (CID). Following fragmentation of the tag amide bond, however, the balance (carbonyl) moiety is lost (neutral loss) while charge is retained by the reporter group fragment. FIG. 6C illustrates the isotopic tagging used to arrive at 4 isobaric combinations with 4 different reporter group masses (left). A mixture of 4 identical peptides each labeled with one member of the multiplex set appears as a single, unresolved precursor ion in MS (identical m/z; middle). Following collision induced dissociation, the 4 reporter group ions appear as distinct masses (114-117 Da; right). All other sequence-informative fragment ions (b-, y- etc.) remain isobaric, and their individual ion current signals (signal intensities) are additive. This remains the case even for those tryptic peptides that are labeled at both the N-terminus and lysine side chains, and those peptides containing internal lysine residues due to incomplete cleavage with trypsin. The relative concentration of the peptides is thus deduced from the relative intensities of the corresponding reporter-ions. Quantitation is performed at the MS/MS stage rather than in MS.

In FIG. 7 an example of an MS/MS spectrum of the peptide TPHPALTEAK from a protein digest mixture prepared by labeling 4 separate digests with each of the 4 isobaric reagents and combining the reaction mixtures in a 1:1:1:1 ratio is shown. The isotopic distribution of the precursor ([M+H]+, m/z 1352.84) is shown in i). Boxed components of the spectrum shown in the middle are shown on the bottom. These components are a low mass region showing the signature ions used for quantitation in ii), isotopic distribution of the b₆ fragment in iii), and isotopic distribution of the Y₇ fragment ion in iv). The peptide is labeled by isobaric tags at both the N-terminus and C-terminal lysine side-chain. The precursor ion and all the internal fragment ions (e.g. type b- and y-) therefore contain all four members of the tag set, but remain isobaric. The example shown is the spectrum obtained from the singly-charged [M+H]+ peptide using a 4700 MALDI TOF-TOF analyzer, but the same holds true for any multiply-charged peptide analyzed with an ESI-source mass spectrometer.

TMT labels can be used as multidimension signals and reporter signals in the disclosed compositions and methods. TMT systems are described in U.S. Application No. 2003/0194717, which is incorporated by reference herein for their descriptions of TMT labels and use of TMT labels for labeling and detecting. TMT, or Tandem Mass Tags, are chemical mass tags which have individual fragmentation patterns in tandem mass spectrometry. TMT labels can be used as multidimension signals and reporter signals in the disclosed compositions and methods. Each TMT in a series comprises a mass reporter group (M) or (M′), a pro-fragmentation linker group (F), a mass normalization group (N) or (N′) and an amine reactive group (M-F-N-(R) First Tag; and M′-F-N′-(R) Second Tag). All members of the series have the same overall mass and physical chemical properties ensuring they co-elute during chromatography and mass spectrometry. When the labeled peptides enter the tandem MS ion beam, the TMT's pro-fragmentation elements are released giving rise to unique mass to charge signals.

C. Indicator Signals

Indicator signals are molecules that have at least one characteristic that allows the indicator signal to be distinguished and/or separated from other multidimension signals. Generally, indicator signals need only be distinguishable and/or separable from other multidimension signals present in same level of analysis. Indicator signals can be used in sets. Thus, for example, a set of indicator signals that differ in some property or characteristic can be used to label different samples and/or analytes. In some forms of indicator signals, the characteristic can be chosen to be compatible with a characteristic of reporter signals and/or other multidimension signals used in the same assay or assay system such that a recognizable pattern will result during analysis of the multidimension signals. For example, indicator signals or sets of indicator signals have masses (or mass-to-charge ratios) different from the mass (or mass-to-charge ratio) of reporter signals and sets of reporter signals can be used in the same assay to generate characteristic patterns of mass (or mass-to-charge ratio) in mass spectrometry.

The disclosed indicator signals are preferably used in sets where members of a set have different mass-to-charge ratios (m/z). In such forms, is also preferred that the indicator signals have different mass-to-charge ratios from other multidimension signals, such as reporter signals, used in the same assay. This facilitates sensitive distinction of indicator signals from each other and from other multidimension signals based on mass-to-charge ratio. Indicator signals can have any structure that allows the generation of patterns with other multidimension signals in analysis of the disclosed methods.

Indicator signals preferably are used in sets where all the indicator signals in the set have different physical properties. The different (or distinguishing) properties allow the indicator signals to be distinguished and/or separated from other multidimension signals differing in one or more of the properties. Preferably, the indicator signals in a set have different mass-to-charge ratios (m/z). That is, the indicator signals in a set are non-isobaric. This allows the indicator signals (and/or the proteins or other analytes to which they are attached) to be separated precisely from other molecules based on mass-to-charge ratio and to generate a pattern of masses with each other and with other multidimension signals. Sets of indicator signals can have any number of indicator signals.

Indicator signals in a set can be, but are preferably not, subunit isomers. However, indicator signals can have a portion that is subunit isomeric to a portion of the other members of the set and a portion that is not subunit isomeric to a portion of the other members of the set. The non-subunit isomeric portion of the indicator signals can then serve as the basis for the difference in properties between members of the set. Thus, for example, a first indicator signal could be the chain, trp-ala-ser-lys-gln, a second indicator signal could be the chain pro-ala-lys-ser-gln, and a third indicator signal could be the chain leu-ser-ala-lys-pro. The first indicator signal and the second indicator signal each have a portion (ala-ser-lys-gln or ala-lys-ser-gln) that is subunit isomeric and a portion (trp or leu) that is not sub unit isomeric with the other. The third indicator signal does not share this subunit isomeric portion. However, all three indicator signals have a subunit isomeric portion (ala-ser-lys, ala-lys-ser or ser-ala-lys).

Selenium substitution can be used to alter the mass of indicator signals. Selenium can substitute for sulfur in methionine, resulting in the modified amino acid selenomethionine. Selenium is approximately forty seven mass units larger than sulfur. Mass spectrometry may be used to identify peptides or proteins incorporating selenomethionine and methionine at a particular ratio. Small proteins and peptides with known selenium/sulfur ratio are preferably produced by chemical synthesis incorporating selenomethionine and methionine at the desired ratio. Larger proteins or peptides may be by produced from an E. coli expression system, or any other expression system that inserts selenomethionine and methionine at the desired ratio (Hendrickson et al., Selenomethionyl proteins producedfor analysis by multiwavelength anomalous diffraction (MAD): a vehicle for direct determination of three-dimensional structure. Embo J, 9(5):1665-72 (1990), Cowie and Cohen, Biosynthesis by Escherichia coli of active altered proteins containing selenium instead of sulfur. Biochimica et Biophysica Acta, 26:252-261 (1957), and Oikawa et al., Metalloselenonein, the selenium analogue of metallothionein: synthesis and characterization of its complex with copper ions. Proc Natl Acad Sci USA, 88(8):3057-9 (1991).

Indicator signal calibrators are a special form of indicator signal characterized by their use in reporter signal calibration. Indicator signal calibrators can be any form of indicator signal, as described above and elsewhere herein, but are used as separate molecules that are not physically associated with analytes or proteins being assessed. Thus, indicator signal calibrators need not (and preferably do not) have reactive groups for coupling to analytes or proteins and need not be (and preferably are not) associated with specific binding molecules or other molecules or components described herein as being associated with indicator signals. Indicator signal calibrators form a predetermined pattern with reporter signal calibrators when used together. In reporter signal calibration, reporter signal calibrators preferably share one or more common properties with one or more analytes while indicator signal calibrators preferably do not. Rather, the indicator signal calibrators serve to generate a pattern with the reporter signal calibrators.

ICAT labels can be used as multidimension signals and indicator signals in the disclosed compositions and methods. ICAT systems and reagents (labels) are described in PCT Application No. WO00/011208, and examples of using ICAT systems can be found in PCT Application No. WO02/090929 and U.S. Application No. 2002/0192720, each of which are incorporated by reference herein for their descriptions of ICAT labels and use of ICAT labels for labeling and detecting. ICAT labels are designed to affinity isolate and quantify via the use of a stable isotope the relative concentrations of cysteine-containing tryptic peptides obtained from digests of control versus experimental samples. In one embodiment, the ICAT reagent has a thiol-specific reactive group adjacent to an alkyl linker, which contains either nine [¹²C] or nine [¹³C] atoms—thus resulting in a mass difference of 9 daltons between the control versus the corresponding experimental version of the same tryptic peptide. The alkyl linker in the ICAT reagent is connected to a (cleavable) biotin group which allows rapid affinity isolation of cysteine-containing tryptic peptides.

Mass defect tags can be used as multidimension signals and indicator signals in the disclosed compositions and methods. Mass defect tags and their use are described in U.S. Application No. 2002/0172961 and Hall et al., J. Mass Spectrometry 38:809-816 (2003), both of which are incorporated by reference herein for their descriptions of mass defect tags and use of mass defect tags for labeling and detecting. Mass defect tags use elements that have a larger mass defect which results in mass spectrometry ion species with masses that fall between the masses of ion species having integer or near integer mass differences. Mass spectrometry peaks having such non-integer masses can thus be identified as labeled species and distinguished from other peaks. As with other mass labels, the characteristic mass of molecules labeled with mass defect tags can contribute to a predetermined pattern used to in an indicator level of analysis.

Other labels that can be used as multidimension signals and/or indicator signals are described in U.S. Application Nos. 2004/0018565, 2003/0100018, 2003/0050453, 2004/0023274, 2002/014673, 2003/0022225, and U.S. Pat. Nos. 6,312,893, 6,312,904, 6,629,040, and Geysen et al. (Chemistry & Biology 3(8):679-688 (1996)), all of which are incorporated by reference herein.

D. Analytes and Proteins

The disclosed methods make use of analytes and proteins generally as objects of detection, measurement and/or analysis. Analytes can be any molecule or portion of a molecule that is to be detected, measured, or otherwise analyzed. A “protein” is a type of analyte and, in accordance with the invention, includes proteins, peptides, and fragments of proteins or peptides. An analyte or protein need not be a physically separate molecule, but may be a part of a larger molecule. Analytes include biological molecules, organic molecules, chemicals, compositions, and any other molecule or structure to which the disclosed method can be adapted. It should be understood that different forms of the disclosed method are more suitable for some types of analytes than other forms of the method. Analytes are also referred to as target molecules.

Preferred analytes are biological molecules. Biological molecules include but are not limited to proteins, peptides, enzymes, amino acid modifications, protein domains, protein motifs, nucleic acid molecules, nucleic acid sequences, DNA, RNA, mRNA, cDNA, metabolites, carbohydrates, and nucleic acid motifs. As used herein, “biological molecule” and “biomolecule” refer to any molecule or portion of a molecule or multi-molecular assembly or composition, that has a biological origin, is related to a molecule or portion of a molecule or multi-molecular assembly or composition that has a biological origin. Biomolecules can be completely artificial molecules that are related to molecules of biological origin.

E. Samples

Any sample from any source can be used with the disclosed method. In general, analyte samples should be samples that contain, or may contain, analytes. In general, protein samples should be samples that contain, or may contain, protein molecules. Examples of suitable analyte and protein samples include cell samples, tissue samples, cell extracts, components or fractions purified from another sample, environmental samples, biofilm samples, culture samples, tissue samples, bodily fluids, and biopsy samples. Numerous other sources of samples are known or can be developed and any can be used with the disclosed method. Preferred protein samples for use with the disclosed method are samples of cells and tissues. Protein samples can be complex, simple, or anywhere in between. For example, a protein sample may include a complex mixture of proteins (a tissue sample, for example), a protein sample may be a highly purified protein preparation, or a single type of protein. Likewise, an analyte sample may include a complex mixture of biological molecules (a tissue sample, for example), an analyte sample may be a highly purified protein preparation, or a single type of molecule.

F. Multidimension Molecules

Multidimension molecules (or multidimension signal molecules) are molecules that combine a multidimension signal with a specific binding molecule or decoding tag. Preferably, the multidimension signal and specific binding molecule or decoding tag are covalently coupled or tethered to each other. As used herein, molecules are coupled when they are covalent joined, directly or indirectly. One form of indirect coupling is via a linker molecule. The multidimension signal can be coupled to the specific binding molecule or decoding tag by any of several established coupling reactions. For example, Hendrickson et al., Nucleic Acids Res., 23(3):522-529 (1995) describes a suitable method for coupling oligonucleotides to antibodies. Reporter molecules are molecules that combine a reporter signal with a specific binding molecule or decoding tag. Indicator molecules are molecules that combine an indicator signal with a specific binding molecule or decoding tag. Reporter molecules and indicator molecules are forms of multidimension molecules.

One form of reporter molecule has a peptide nucleic acid as the decoding tag and a multidimension signal peptide as the multidimension signal. The peptide nucleic acid can associate with, for example, an oligonucleotide coding tag, thus associating the multidimension signal peptide with the coding tag. As described elsewhere herein, coding tags can be used to labeled analytes and other molecules.

As used herein, a molecule is said to be tethered to another molecule when a loop of (or from) one of the molecules passes through a loop of (or from) the other molecule. The two molecules are not covalently coupled when they are tethered. Tethering can be visualized by the analogy of a closed loop of string passing through the hole in the handle of a mug. In general, tethering is designed to allow one or both of the molecules to rotate freely around the loop.

G. Specific Binding Molecules

A specific binding molecule is a molecule that interacts specifically with a particular molecule or moiety. The molecule or moiety that interacts specifically with a specific binding molecule is referred to herein as an analyte, such as an analyte. Preferred analytes are analytes. It is to be understood that the term analyte refers to both separate molecules and to portions of such molecules, such as an epitope of a protein, that interacts specifically with a specific binding molecule. Antibodies, either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are examples of specific binding molecules, useful as the affinity portion of a multidimension molecule.

A specific binding molecule that interacts specifically with a particular analyte is said to be specific for that analyte. For example, where the specific binding molecule is an antibody that associates with a particular antigen, the specific binding molecule is said to be specific for that antigen. The antigen is the analyte. A multidimension molecule containing the specific binding molecule can also be referred to as being specific for a particular analyte. Specific binding molecules preferably are antibodies, ligands, binding proteins, receptor proteins, haptens, aptamers, carbohydrates, synthetic polyamides, peptide nucleic acids, or oligonucleotides. Preferred binding proteins are DNA binding proteins. Preferred DNA binding proteins are zinc finger motifs, leucine zipper motifs, helix-turn-helix motifs. These motifs can be combined in the same specific binding molecule.

Antibodies useful as the affinity portion of multidimension molecules, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

One form of specific binding molecule is an oligonucleotide or oligonucleotide derivative. Such specific binding molecules are designed for and used to detect specific nucleic acid sequences. Thus, the analyte for oligonucleotide specific binding molecules are nucleic acid sequences. The analyte can be a nucleotide sequence within a larger nucleic acid molecule. An oligonucleotide specific binding molecule can be any length that supports specific and stable hybridization between the multidimension molecule and the analyte. For this purpose, a length of 10 to 40 nucleotides is preferred, with an oligonucleotide specific binding molecule 16 to 25 nucleotides long being most preferred. It is preferred that the oligonucleotide specific binding molecule is peptide nucleic acid. Peptide nucleic acid forms a stable hybrid with DNA. This allows a peptide nucleic acid specific binding molecule to remain firmly adhered to the target sequence during subsequent amplification and detection operations.

This useful effect can also be obtained with oligonucleotide specific binding molecules by making use of the triple helix chemical bonding technology described by Gasparro et al., Nucleic Acids Res., 22(14):2845-2852 (1994). Briefly, the oligonucleotide specific binding molecule is designed to form a triple helix when hybridized to a target sequence. This is accomplished generally as known, preferably by selecting either a primarily homopurine or primarily homopyrimidine target sequence. The matching oligonucleotide sequence which constitutes the specific binding molecule will be complementary to the selected target sequence and thus be primarily homopyrimidine or primarily homopurine, respectively. The specific binding molecule (corresponding to the triple helix probe described by Gasparro et al.) contains a chemically linked psoralen derivative. Upon hybridization of the specific binding molecule to a target sequence, a triple helix forms. By exposing the triple helix to low wavelength ultraviolet radiation, the psoralen derivative mediates cross-linking of the probe to the target sequence.

H. Multidimension Signal Fusions

Multidimension signal fusions are multidimension signal peptides joined with a protein or peptide of interest in a single amino acid segment (that is, a fusion protein). Such fusions of proteins and peptides of interest with multidimension signal peptides can be expressed as a fusion protein or peptide from a nucleic acid molecule encoding the amino acid segment that constitutes the fusion. A multidimension signal fusion nucleic acid molecule or multidimension signal nucleic acid segment refers to a nucleic acid molecule or nucleic acid sequence, respectively, that encodes a multidimension signal fusion.

The multidimension signal peptide and the protein of interest involved in a multidimension signal fusion need not be directly fused. That is, other amino acids, amino acid sequences, and/or peptide elements can intervene. For example, an epitope tag, if present, can be located between the protein of interest and the multidimension signal peptide in a multidimension signal fusion. The multidimension signal peptide(s) can be fused to a protein in any arrangement, such as at the N-terminal end of the protein, at the C-terminal end of the protein, in or at domain junctions, or at any other appropriate location in the protein. In some forms of the method, it is desirable that the protein remain functional. In such cases, terminal fusions or inter-domain fusions are preferable. Those of skill in the art of protein fusions generally know how to design fusions where the protein of interest remains functional. In other embodiments, it is not necessary that the protein remain functional in which case the multidimension signal peptide and protein can have any desired structural organization.

A given multidimension signal fusion can include one or more multidimension signal peptides and one or more proteins or peptides of interest. In addition, multidimension signal fusions can include one or more amino acids, amino acid sequences, and/or peptide elements. The disclosed multidimension signal fusions comprise a single, contiguous polypeptide chain. Thus, although multiple amino acid segments can be part of the same contiguous polypeptide chain, all of the components (that is, the multidimension signal peptide(s) and protein(s) and peptide(s) of interest) of a given amino acid segment are part of the same contiguous polypeptide chain.

In preferred embodiments, multidimension signal peptides, multidimension signal fusions (or amino acid segments), nucleic acid segments encoding multidimension signal fusions, and/or nucleic acid molecules comprising nucleic acid segments encoding multidimension signal fusions are used in sets where the multidimension signal peptides, the multidimension signal fusions, and/or subsegments of the multidimension signal fusions constituting or present in the set have similar properties (such as similar mass-to-charge ratios). The similar properties allow the multidimension signals, the multidimension signal fusions, or subsegments of the multidimension signal fusions to be distinguished and/or separated from other molecules lacking one or more of the properties. Preferably, the multidimension signals, the multidimension signal fusions, or subsegments of the multidimension signal fusions constituting or present in a set have the same mass-to-charge ratio (m/z). That is, the multidimension signals, the multidimension signal fusions, or subsegments of the multidimension signal fusions in a set can be isobaric. This allows the multidimension signals, the multidimension signal fusions, or subsegments of the multidimension signal fusions to be separated precisely from other molecules based on mass-to-charge ratio. The result of the filtering is a huge increase in the signal to noise ratio (S/N) for the system, allowing more sensitive and accurate detection.

Sets of multidimension signal fusions (also referred to as amino acid segments), multidimension signal fusion fragments (also referred to as subsegments of the multidimension signal fusions or amino acid subsegments), multidimension signal peptides, nucleic acid segments encoding multidimension signal fusions, or nucleic acid molecules comprising nucleic acid segments encoding multidimension signal fusions can have any number of multidimension signal fusions, multidimension signal fusion fragments, multidimension signal peptides, nucleic acid segments encoding multidimension signal fusions, or nucleic acid molecules comprising nucleic acid segments encoding multidimension signal fusions. For example, sets of multidimension signal fusions, multidimension signal fusion fragments, multidimension signal peptides, nucleic acid segments encoding multidimension signal fusions, or nucleic acid molecules comprising nucleic acid segments encoding multidimension signal fusions can have one, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, three hundred or more, four hundred or more, or five hundred or more different multidimension signal fusions, multidimension signal fusion fragments, multidimension signal peptides, nucleic acid segments encoding multidimension signal fusions, or nucleic acid molecules comprising nucleic acid segments encoding multidimension signal fusions. Although specific numbers of multidimension signal fusions, multidimension signal fusion fragments, multidimension signal peptides, nucleic acid segments encoding multidimension signal fusions, and nucleic acid molecules comprising nucleic acid segments encoding multidimension signal fusions, and specific endpoints for ranges of the number of multidimension signal fusions, multidimension signal fusion fragments, multidimension signal peptides, nucleic acid segments encoding multidimension signal fusions, and nucleic acid molecules comprising nucleic acid segments encoding multidimension signal fusions, are recited, each and every specific number of multidimension signal fusions, multidimension signal fusion fragments, multidimension signal peptides, nucleic acid segments encoding multidimension signal fusions, and nucleic acid molecules comprising nucleic acid segments encoding multidimension signal fusions, and each and every specific endpoint of ranges of numbers of multidimension signal fusions, multidimension signal fusion fragments, multidimension signal peptides, nucleic acid segments encoding multidimension signal fusions, and nucleic acid molecules comprising nucleic acid segments encoding multidimension signal fusions, are specifically contemplated, although not explicitly listed, and each and every specific number of multidimension signal fusions, multidimension signal fusion fragments, multidimension signal peptides, nucleic acid segments encoding multidimension signal fusions, and nucleic acid molecules comprising nucleic acid segments encoding multidimension signal fusions, and each and every specific endpoint of ranges of numbers of multidimension signal fusions, multidimension signal fusion fragments, multidimension signal peptides, nucleic acid segments encoding multidimension signal fusions, and nucleic acid molecules comprising nucleic acid segments encoding multidimension signal fusions, are hereby specifically described.

Multidimension signal fusions can be expressed in any suitable manner. For example, nucleic acid sequences and nucleic acid segments encoding multidimension signal fusions can be expressed in vitro, in cells, and/or in cells in organism. Many techniques and systems for expression of nucleic acid sequences and proteins are known and can be used with the disclosed multidimension signal fusions. For example, many expression sequences, vector systems, transformation and transfection techniques, and transgenic organism production methods are known and can be used with the disclosed multidimension signal peptide method and compositions. Systems are known for integration of nucleic acid constructs into chromosomes of cells and organisms (see, for example, Groth et al. (2000) A phage integrase directs efficient site-specific integration in human cells. Proc Natl Acad Sci USA 97:5995-6000; Hong et al. (2001) Development of two bacterial artificial chromosome shuttle vectors for a recombination-based cloning and regulated expression of large genes in mammalian cells. Analytical Biochemistry 291:142-148) which can be used with the disclosed nucleic acid molecules and segments encoding multidimension signal fusions or to form nucleic acid segment encoding multidimension signal fusions.

As used herein, an expression sample is a sample that contains, or might contain, one or more multidimension signal fusions expressed from a nucleic acid molecule. An expression sample to be analyzed can be subjected to fractionation or separation to reduce the complexity of the samples. Fragmentation and fractionation can also be used together in the same assay. Such fragmentation and fractionation can simplify and extend the analysis of the expression.

Nucleic acid molecules encoding multidimension signal fusions can be used in sets where the multidimension signal peptides in the multidimension signal fusions encoded by a set of nucleic acid molecules can have one or more common properties that allow the multidimension signal peptides to be separated or distinguished from molecules lacking the common property. Similarly, nucleic acid molecules encoding amino acid segments can be used in sets where the multidimension signal peptides in the amino acid segments encoded by a set of nucleic acid molecules can have one or more common properties that allow the multidimension signal peptides to be separated or distinguished from molecules lacking the common property. Nucleic acid molecules encoding amino acid segments can be used in sets where the amino acid segments encoded by a set of nucleic acid molecules can have one or more common properties that allow the amino acid segments to be separated or distinguished from molecules lacking the common property.

Likewise, nucleic acid molecules encoding multidimension signal fusions can be used in sets where the multidimension signal peptides in the multidimension signal fusions encoded by a set of nucleic acid molecules can have one or more properties that generate a pattern in an indicator level of analysis. Similarly, nucleic acid segments (which, generally, are part of nucleic acid molecules) encoding multidimension signal fusions can be used in sets where the multidimension signal peptides in the multidimension signal fusions encoded by a set of nucleic acid segments can have one or more properties that generate a pattern. Other relationships between members of the sets of nucleic acid molecules, nucleic acid segments, amino acid segments, multidimension signal peptides, and proteins of interest are contemplated.

Nucleic acid segments (which, generally, are part of nucleic acid molecules) encoding multidimension signal fusions can be used in sets where the multidimension signal peptides in the multidimension signal fusions encoded by a set of nucleic acid segments can have one or more common properties that allow the multidimension signal peptides to be separated or distinguished from molecules lacking the common property. Similarly, nucleic acid segments encoding amino acid segments can be used in sets where the multidimension signal peptides in the amino acid segments encoded by a set of nucleic acid molecules can have one or more common properties that allow the multidimension signal peptides to be separated or distinguished from molecules lacking the common property. Nucleic acid segments encoding amino acid segments can be used in sets where the amino acid segments encoded by a set of nucleic acid molecules can have one or more common properties that allow the amino acid segments to be separated or distinguished from molecules lacking the common property. Other relationships between members of the sets of nucleic acid molecules, nucleic acid segments, amino acid segments, multidimension signal peptides, and proteins of interest are contemplated.

I. Multidimension Signal/Analyte Conjugates

Compositions where multidimension signals are associated with, incorporated into, or otherwise linked to the analytes or proteins are referred to as multidimension signal/analyte conjugates (or MDS/analyte conjugates) or multidimension signal/protein conjugates (or MDS/protein conjugates). Such conjugates include multidimension signals associated with analytes, such as a multidimension signal probe hybridized to a nucleic acid sequence; multidimension signals covalently coupled to analytes, such as multidimension signals linked to proteins via a linking group; and multidimension signals incorporated into analytes, such as fusions between a protein of interest and a multidimension signal peptide (or peptide multidimension signal).

In some embodiments of the disclosed methods employing multidimension signals, the multidimension signals can be altered such that the altered forms of different multidimension signals can be distinguished from each other. Multidimension signal/analyte conjugates can be altered, generally through alteration of the multidimension signal portion of the conjugate, such that the altered forms of different multidimension signals, altered forms of different multidimension signal/analyte conjugates, or both, can be distinguished from each other. Where the multidimension signal or multidimension signal/analyte conjugate is altered by fragmentation, any, some, or all of the fragments can be distinguished from each other, depending on the embodiment. For example, where multidimension signals fragmented into two parts, either or both parts of the multidimension signals can be distinguished. Where multidimension signal/analyte conjugates are fragmented into two parts (with the break point in the multidimension signal portion), either the multidimension signal fragment, the multidimension signal/analyte fragment, or both can be distinguished. In some embodiments, only one part of a fragmented multidimension signal will be detected and so only this part of the reported signals need be distinguished.

Sets of multidimension signal/analyte conjugates can be used where two or more of the multidimension signal/analyte conjugates in a set have one or more common properties that allow the multidimension signal/analyte conjugates having the common property to be distinguished and/or separated from other molecules lacking the common property. In still other embodiments, analytes can be fragmented (prior to or following conjugation) to produce multidimension signal/analyte fragment conjugates (which can be referred to as fragment conjugates). In such cases, sets of fragment conjugates can be used where two or more of the fragment conjugates in a set have one or more common properties that allow the fragment conjugates having the common property to be distinguished and/or separated from other molecules lacking the common property. It should be understood that fragmented analytes can be considered analytes in their own right. In this light, reference to fragmented analytes is made for convenience and clarity in describing certain embodiments and to allow reference to both the base analyte and the fragmented analyte.

Sets of multidimension signal/analyte conjugates or multidimension signal/analyte fragment conjugates (fragment conjugates) can have any number of multidimension signal/analyte conjugates or multidimension signal/analyte fragment conjugates. For example, sets of multidimension signal/analyte conjugates or multidimension signal/analyte fragment conjugates can have one, two or more, three or more, four or more, five or more, six or more, seven or more, eight or more, nine or more, ten or more, twenty or more, thirty or more, forty or more, fifty or more, sixty or more, seventy or more, eighty or more, ninety or more, one hundred or more, two hundred or more, three hundred or more, four hundred or more, or five hundred or more different multidimension signal/analyte conjugates or multidimension signal/analyte fragment conjugates. Although specific numbers of multidimension signal/analyte conjugates and multidimension signal/analyte fragment conjugates, and specific endpoints for ranges of the number of multidimension signal/analyte conjugates and multidimension signal/analyte fragment conjugates, are recited, each and every specific number of multidimension signal/analyte conjugates and multidimension signal/analyte fragment conjugates, and each and every specific endpoint of ranges of numbers of multidimension signal/analyte conjugates and multidimension signal/analyte fragment conjugates, are specifically contemplated, although not explicitly listed, and each and every specific number of multidimension signal/analyte conjugates and multidimension signal/analyte fragment conjugates, and each and every specific endpoint of ranges of numbers of multidimension signal/analyte conjugates and multidimension signal/analyte fragment conjugates, are hereby specifically described.

As indicated above, multidimension signals conjugated with analytes or proteins can be altered while in the conjugate and distinguished. Conjugated multidimension signals can also be dissociated or separated, in whole or in part, from the conjugated analytes prior to their alteration. Other conjugated multidimension signals can also be dissociated or separated, in whole or in part, from the conjugated analytes prior to analysis. Where the multidimension signals are dissociated (in whole or in part) from the analytes, the method can be performed such that the fact of association between the analyte and multidimension signal is part of the information obtained when the multidimension signal is detected. In other words, the fact that the multidimension signal may be dissociated from the analyte for detection does not obscure the information that the detected multidimension signal was associated with the analyte.

As used herein, multidimension signal conjugate refers both to multidimension signal/analyte conjugates and to other components of the disclosed method such as multidimension molecules.

As with multidimension signals generally, multidimension signal/analyte conjugates and multidimension signal/analyte fragment conjugates can be used in sets where the multidimension signal/analyte conjugates or fragment conjugates in a set can have one or more common properties that allow the multidimension signal/analyte conjugates or fragment conjugates to be separated or distinguished from molecules lacking the common property.

J. Capture Arrays

A capture array (also referred to herein as an array) includes a plurality of capture tags immobilized on a solid-state substrate, preferably at identified or predetermined locations on the solid-state substrate. In this context, plurality of capture tags refers to a multiple capture tags each having a different structure. Preferably, each predetermined location on the array (referred to herein as an array element) has one type of capture tag (that is, all the capture tags at that location have the same structure). Each location will have multiple copies of the capture tag. The spatial separation of capture tags of different structure in the array allows separate detection and identification of analytes that become associated with the capture tags. If a decoding tag is detected at a given location in a capture array, it indicates that the analyte corresponding to that array element was present in the target sample.

Solid-state substrates for use in capture arrays can include any solid material to which capture tags can be coupled, directly or indirectly. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, disks, compact disks, fibers, optical fibers, woven fibers, shaped polymers, particles and microparticles. A preferred form for a solid-state substrate is a compact disk.

Although preferred, it is not required that a given capture array be a single unit or structure. The set of capture tags may be distributed over any number of solid supports. For example, at one extreme, each capture tag may be immobilized in a separate reaction tube or container. Arrays may be constructed upon non permeable or permeable supports of a wide variety of support compositions such as those described above. The array spot sizes and density of spot packing vary over a tremendous range depending upon the process(es) and material(s) used.

Methods for immobilizing antibodies and other proteins to substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

Methods for immobilization of oligonucleotides to solid-state substrates are well established. Oligonucleotide capture tags can be coupled to substrates using established coupling methods. For example, suitable attachment methods are described by Pease et al., Proc. Natl. Acad. Sci. USA 91(11):5022-5026 (1994), Khrapko et al., Mol Biol (Mosk) (USSR) 25:718-730 (1991), U.S. Pat. No. 5,871,928 to Fodor et al., U.S. Pat. No. 5,654,413 to Brenner, U.S. Pat. No. 5,429,807, and U.S. Pat. No. 5,599,695 to Pease et al. A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994).

Planar array technology has been utilized for many years (Shalon, D., S. J. Smith, and P. O. Brown, A DNA microarray system for analyzing complex DNA samples using two-color fluorescent probe hybridization. Genome Res, 1996. 6(7): p. 639-45, Singh-Gasson, S., et al., Maskless fabrication of light-directed oligonucleotide microarrays using a digital micromirror array. Nat Biotechnol, 1999. 17(10): p. 974-8, Southern, E. M., U. Maskos, and J. K. Elder, Analyzing and comparing nucleic acid sequences by hybridization to arrays of oligonucleotides: evaluation using experimental models. Genomics, 1992. 13(4): p. 1008-17, Nizetic, D., et al., Construction, arraying, and high-density screening of large insert libraries of human chromosomes X and 21: their potential use as reference libraries. Proc Natl Acad Sci USA, 1991. 88(8): p. 3233-7, Van Oss, C. J., R. J. Good, and M. K. Chaudhury, Mechanism of DNA (Southern) and protein (Western) blotting on cellulose nitrate and other membranes. J Chromatogr, 1987. 391(1): p. 53-65, Ramsay, G., DNA chips: state-of-the art. Nat Biotechnol, 1998. 16(1): p. 40-4, Schena, M., et al., Parallel human genome analysis: microarray-based expression monitoring of 1000 genes. Proc Natl Acad Sci USA, 1996. 93(20): p. 10614-9, Lipshutz, R. J., et al., High density synthetic oligonucleotide arrays. Nat Genet, 1999. 21(1 Suppl): p. 20-4, Pease, A. C., et al., Light-generated oligonucleotide arrays for rapid DNA sequence analysis. Proc Natl Acad Sci USA, 1994. 91(11): p. 5022-6, Maier, E., et al., Application of robotic technology to automated sequence fingerprint analysis by oligonucleotide hybridisation. J Biotechnol, 1994. 35(2-3): p. 191-203, Vasiliskov, A. V., et al., Fabrication of microarray of gel-immobilized compounds on a chip by copolymerization. Biotechniques, 1999. 27(3): p. 592-4, 596-8, 600 passim, and Yershov, G., et al., DNA analysis and diagnostics on oligonucleotide microchips. Proc Natl Acad Sci USA, 1996. 93(10): p. 4913-8).

Oligonucleotide capture tags in arrays can also be designed to have similar hybrid stability. This would make hybridization of fragments to such capture tags more efficient and reduce the incidence of mismatch hybridization. The hybrid stability of oligonucleotide capture tags can be calculated using known formulas and principles of thermodynamics (see, for example, Santa Lucia et al., Biochemistry 35:3555-3562 (1996); Freier et al., Proc. Natl. Acad. Sci. USA 83:9373-9377 (1986); Breslauer et al., Proc. Natl. Acad. Sci. USA 83:3746-3750 (1986)). The hybrid stability of the oligonucleotide capture tags can be made more similar (a process that can be referred to as smoothing the hybrid stabilities) by, for example, chemically modifying the capture tags (Nguyen et al., Nucleic Acids Res. 25(15):3059-3065 (1997); Hohsisel, Nucleic Acids Res. 24(3):430-432 (1996)). Hybrid stability can also be smoothed by carrying out the hybridization under specialized conditions (Nguyen et al., Nucleic Acids Res. 27(6):1492-1498 (1999); Wood et al., Proc. Natl. Acad. Sci. USA 82(6):1585-1588 (1985)).

Another means of smoothing hybrid stability of the oligonucleotide capture tags is to vary the length of the capture tags. This would allow adjustment of the hybrid stability of each capture tag so that all of the capture tags had similar hybrid stabilities (to the extent possible). Since the addition or deletion of a single nucleotide from a capture tag will change the hybrid stability of the capture tag by a fixed increment, it is understood that the hybrid stabilities of the capture tags in a capture array will not be equal. For this reason, similarity of hybrid stability as used herein refers to any increase in the similarity of the hybrid stabilities of the capture tags (or, put another way, any reduction in the differences in hybrid stabilities of the capture tags).

The efficiency of hybridization and ligation of oligonucleotide capture tags to sample fragments can also be improved by grouping capture tags of similar hybrid stability in sections or segments of a capture array that can be subjected to different hybridization conditions. In this way, the hybridization conditions can be optimized for particular classes of capture tags.

K. Capture Tags

A capture tag is any compound that can be used to capture or separate compounds or complexes having the capture tag. Preferably, a capture tag is a compound that interacts specifically with a particular molecule or moiety. Preferably, the molecule or moiety that interacts specifically with a capture tag is an analyte. It is to be understood that the term analyte refers to both separate molecules and to portions of such molecules, such as an epitope of a protein, that interacts specifically with a capture tag. Antibodies, either member of a receptor/ligand pair, synthetic polyamides (Dervan and Burli, Sequence-specific DNA recognition bypolyamides. Curr Opin Chem Biol, 3(6):688-93 (1999); Wemmer and Dervan, Targeting the minor groove of DNA. Curr Opin Struct Biol, 7(3):355-61 (1997)), nucleic acid probes, and other molecules with specific binding affinities are examples of capture tags.

A capture tag that interacts specifically with a particular analyte is said to be specific for that analyte. For example, where the capture tag is an antibody that associates with a particular antigen, the capture tag is said to be specific for that antigen. The antigen is the analyte. Capture tags preferably are antibodies, ligands, binding proteins, receptor proteins, haptens, aptamers, carbohydrates, synthetic polyamides, peptide nucleic acids, or oligonucleotides. Preferred binding proteins are DNA binding proteins. Preferred DNA binding proteins are zinc finger motifs, leucine zipper motifs, helix-turn-helix motifs. These motifs can be combined in the same capture tag.

Antibodies useful as the affinity portion of multidimension molecules can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection ofzincfingers with altered DNA-binding specificity. Biochemistry, 33(19):5689-95 (1994), Chandrasegaran and Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

One form of capture tag is an oligonucleotide or oligonucleotide derivative. Such capture tags are designed for and used to detect specific nucleic acid sequences. Thus, the analyte for oligonucleotide capture tags are nucleic acid sequences. The analyte can be a nucleotide sequence within a larger nucleic acid molecule. An oligonucleotide capture tag can be any length that supports specific and stable hybridization between the capture tag and the analyte. For this purpose, a length of 10 to 40 nucleotides is preferred, with an oligonucleotide capture tag 16 to 25 nucleotides long being most preferred. It is preferred that the oligonucleotide capture tag is peptide nucleic acid. Peptide nucleic acid forms a stable hybrid with DNA. This allows a peptide nucleic acid capture tag to remain firmly adhered to the target sequence during subsequent amplification and detection operations.

This useful effect can also be obtained with oligonucleotide capture tags by making use of the triple helix chemical bonding technology described by Gasparro et al., Nucleic Acids Res., 22(14):2845-2852 (1994). Briefly, the oligonucleotide capture tag is designed to form a triple helix when hybridized to a target sequence. This is accomplished generally as known, preferably by selecting either a primarily homopurine or primarily homopyrimidine target sequence. The matching oligonucleotide sequence which constitutes the capture tag will be complementary to the selected target sequence and thus be primarily homopyrimidine or primarily homopurine, respectively. The capture tag (corresponding to the triple helix probe described by Gasparro et al.) contains a chemically linked psoralen derivative. Upon hybridization of the capture tag to a target sequence, a triple helix forms. By exposing the triple helix to low wavelength ultraviolet radiation, the psoralen derivative mediates cross-linking of the probe to the target sequence.

L. Sample Arrays

A sample array includes a plurality of samples (for example, expression samples, tissue samples, protein samples) immobilized on a solid-state substrate, preferably at identified or predetermined locations on the solid-state substrate. Preferably, each predetermined location on the sample array (referred to herein as a sample array element) has one type of sample. The spatial separation of different samples in the sample array allows separate detection and identification of multidimension signals (or multidimension molecules, multidimension signals, multidimension molecules, indicator signals, indicator molecules, or coding tags) that become associated with the samples. If a multidimension signal is detected at a given location in a sample array, it indicates that the analyte corresponding to that multidimension signal was present in the sample corresponding to that sample array element.

Solid-state substrates for use in sample arrays can include any solid material to which samples can be adhered, directly or indirectly. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, disks, compact disks, fibers, optical fibers, woven fibers, shaped polymers, particles and microparticles. A preferred form for a solid-state substrate is a compact disk.

Although preferred, it is not required that a given sample array be a single unit or structure. The set of samples may be distributed over any number of solid supports. For example, at one extreme, each sample may be immobilized in a separate reaction tube or container. Sample arrays may be constructed upon non permeable or permeable supports of a wide variety of support compositions such as those described above. The array spot sizes and density of spot packing vary over a tremendous range depending upon the process(es) and material(s) used. Methods for adhering or immobilizing samples and samplecomponents to substrates are well established.

A preferred form of sample array is a tissue array, where there are small tissue samples on a substrate. Such tissue microarrays exist, and are used, for example, in a cohort to study breast cancer. The disclosed method can be used, for example, to probe multiple analytes in multiple samples. Sample arrays can be, for example, labeled with different multidimension signals, the whole support then introduced into source region of a mass spec, and sampled by MALDI.

M. Decoding Tags

Decoding tags are any molecule or moiety that can be associated with coding tags, directly or indirectly. Decoding tags are associated with multidimension signals (making up a multidimension molecule) to allow indirect association of the multidimension signals with an analyte. Decoding tags preferably are oligonucleotides, carbohydrates, synthetic polyamides, peptide nucleic acids, antibodies, ligands, proteins, haptens, zinc fingers, aptamers, or mass labels.

Preferred decoding tags are molecules capable of hybridizing specifically to an oligonucleotide coding tag. Most preferred are peptide nucleic acid decoding tags. Oligonucleotide or peptide nucleic acid decoding tags can have any arbitrary sequence. The only requirement is hybridization to coding tags. The decoding tags can each be any length that supports specific and stable hybridization between the coding tags and the decoding tags. For this purpose, a length of 10 to 35 nucleotides is preferred, with a decoding tag 15 to 20 nucleotides long being most preferred.

Multidimension molecules containing decoding tags preferably are capable of being released by matrix-assisted laser desorption-ionization (MALDI) in order to be separated and identified by time-of-flight (TOF) mass spectrometry, or by another detection technique. A decoding tag may be any oligomeric molecule that can hybridize to a coding tag. For example, a decoding tag can be a DNA oligonucleotide, an RNA oligonucleotide, or a peptide nucleic acid (PNA) molecule. Preferred decoding tags are PNA molecules.

N. Coding Tags

Coding tags are molecules or moieties with which decoding tags can associate. Coding tags can be any type of molecule or moiety that can serve as a target for decoding tag association. Preferred coding tags are oligomers, oligonucleotides, or nucleic acid sequences. Coding tags can also be a member of a binding pair, such as streptavidin or biotin, where its cognate decoding tag is the other member of the binding pair. Coding tags can also be designed to associate directly with some types of multidimension signals. For example, oligonucleotide coding tags can be designed to interact directly with peptide nucleic acid multidimension signals (which are multidimension signals composed of peptide nucleic acid).

The oligomeric base sequences of oligomeric coding tags can include RNA, DNA, modified RNA or DNA, modified backbone nucleotide-like oligomers such as peptide nucleic acid, methylphosphonate DNA, and 2′-O-methyl RNA or DNA. Oligomeric or oligonucleotide coding tags can have any arbitrary sequence. The only requirement is association with decoding tags (preferably by hybridization). In the disclosed method, multiple coding tags can become associated with a single analyte. The context of these multiple coding tags depends upon the technique used for signal amplification. Thus, where branched DNA is used, the branched DNA molecule includes the multiple coding tags on the branches. Where oligonucleotide dendrimers are used, the coding tags are on the dendrimer arms. Where rolling circle replication is used, multiple coding tags result from the tandem repeats of complement of the amplification target circle sequence (which includes at least one complement of the coding tag sequence). In this case, the coding tags are tandemly repeated in the tandem sequence DNA.

Oligonucleotide coding tags can each be any length that supports specific and stable hybridization between the coding tags and the decoding tags. For this purpose, a length of 10 to 35 nucleotides is preferred, with a coding tag 15 to 20 nucleotides long being most preferred.

The branched DNA for use in the disclosed method is generally known (Urdea, Biotechnology 12:926-928 (1994), and Horn et al., Nucleic Acids Res 23:4835-4841 (1997)). As used herein, the tail of a branched DNA molecule refers to the portion of a branched DNA molecule that is designed to interact with the analyte. The tail is a specific binding molecule. In general, each branched DNA molecule should have only one tail. The branches of the branched DNA (also referred to herein as the arms of the branched DNA) contain coding tag sequences. Oligonucleotide dendrimers (or dendrimeric DNA) are also generally known (Shchepinov et al., Nucleic Acids Res. 25:4447-4454 (1997), and Orentas et al., J. Virol. Methods 77:153-163 (1999)). As used herein, the tail of an oligonucleotide dendrimer refers to the portion of a dendrimer that is designed to interact with the analyte. In general, each dendrimer should have only one tail. The dendrimeric strands of the dendrimer are referred to herein as the arms of the oligonucleotide dendrimer and contain coding tag sequences.

Coding tags can be coupled (directly or via a linker or spacer) to analytes or other molecules to be labeled. Coding tags can also be associated with analytes and other molecules to be labeled. For this purpose, coding molecules are preferred. Coding molecules are molecules that can interact with an analyte and with a decoding tag. Coding molecules include a specific binding molecule and a coding tag. Specific binding molecules are described above.

O. Multidimension Carriers and Coding Carriers

Multidimension carriers are associations of one or more specific binding molecules, a carrier, and a plurality of multidimension signals. Multidimension carriers are used in the disclosed method to associate a large number of multidimension signals with an analyte. Coding carriers are associations of one or more specific binding molecules, a carrier, and a plurality of coding tags. Coding carriers are used in the disclosed method to associate a large number of coding tags with an analyte. The carrier can be any molecule or structure that facilitates association of many multidimension signals with a specific binding molecule. Examples include liposomes, microparticles, nanoparticles, virons, phagmids, and branched polymer structures. A general class of carriers are structures and materials designed for drug delivery. Many such carriers are known. Liposomes are a preferred form of carrier.

Liposomes are artificial structures primarily composed of phospholipid bilayers. Cholesterol and fatty acids may also be included in the bilayer construction. In some forms of the disclosed method, liposomes serve as carriers for arbitrary multidimension signals or coding tags. By combining liposome multidimension carriers, loaded with arbitrary signals or tags, with methods capable of separating a very large multiplicity of signals and tags, it becomes possible to perform highly multiplexed assays.

Liposomes, preferably unilamellar vesicles, are made using established procedures that result in the loading of the interior compartment with a very large number (several thousand) of multidimension signals or coding tag molecules, where the chemical nature of these molecules is well suited for detection by a preselected detection method. One specific type of multidimension signal or coding tag preferably is used for each specific type of liposome carrier.

Each specific type of liposome multidimension or coding carrier is associated with a specific binding molecule. The association may be direct or indirect. An example of a direct association is a liposome containing covalently coupled antibodies on the surface of the phospholipid bilayer. An alternative, indirect association composition is a liposome containing covalently coupled DNA oligonucleotides of arbitrary sequence on its surface; these oligonucleotides are designed to recognize, by base complementarity, specific multidimension molecules. The multidimension molecule may comprise an antibody-DNA covalent complex, whereby the DNA portion of this complex can hybridize specifically with the complementary sequence on a liposome multidimension carrier. In this fashion, the liposome multidimension carrier becomes a generic reagent, which may be associated indirectly with any desired binding molecule.

The use of liposome multidimension carriers can be illustrated with the following example.

1. Liposomes (preferably unilamellar vesicles with an average diameter of 150 to 300 nanometers) are prepared using the extrusion method (Hope et al., Biochimica et Biophysica Acta, 812:55-65 (1985); MacDonald et al., Biochimica et Biophysica Acta, 1061:297-303 (1991)). Other methods for liposome preparation may be used as well.

2. A solution of an oligopeptide, at a concentration 400 micromolar, is used during the preparation of the liposomes, such that the inner volume of the liposomes is loaded with this specific oligopeptide, which will serve to identify a specific analyte of interest. A liposome with an internal diameter of 200 nanometers will contain, on the average, 960 molecules of the oligopeptide. Three separate preparations of liposomes are extruded, each loaded with a different oligopeptide. The oligopeptides are chosen such that they have the same mass-to-charge ratio but will break into fragments with different mass-to-charge ratios such that they will be readily separable by mass spectrometry.

3. The outer surface of the three liposome preparations is conjugated with specific antibodies, as follows: a) the first liposome preparation is reacted with an antibody specific for the p53 tumor suppressor; b) the second liposome preparation is reacted with an antibody specific for the Bcl-2 oncoprotein; c) the third liposome preparation is reacted with an antibody specific or the Her2/neu membrane receptor. Coupling reactions are performed using standard procedures for the covalent coupling of antibodies to molecules harboring reactive amino groups (Hendrickson et al., Nucleic Acids Research, 23:522-529 (1995); Hermanson, Bioconjugate techniques, Academic Press, pp.528-569 (1996); Scheffold et al., Nature Medicine 1:107-110 (2000)). In the case of the liposomes, the reactive amino groups are those present in the phosphatidyl ethanolamine moieties of the liposomes.

4. A glass slide bearing a standard formaldehyde-fixed histological section is contacted with a mixture of all three liposome preparations, suspended in a buffer containing 30 mM Tris-HCl, pH 7.6, 100 mM Sodium Chloride, 1 mM EDTA, 0.1% Bovine serum albumin, in order to allow association of the liposomes with the corresponding protein antigens present in the fixed tissue. After a one hour incubation, the slides are washed twice, for 5 minutes, with the same buffer (30 mM Tris-HCl, pH 7.6, 100 mM Sodium Chloride, 1 mM EDTA, 0.1% Bovine serum albumin). The slides are dried with a stream of air.

5. The slides are coated with a thin layer of matrix solution consisting of 10 mg/ml alpha-cyano-4-hydroxycinnamic acid, 0.1% trifluoroacetic acid in a 50:50 mixture of acetonitrile in water. The slides are dried with a stream of air.

6. The slide is placed on the surface of a MALDI plate, and introduced in a mass spectrometer such as that described in Loboda et al., Design and Performance of a MALDI-QqTOF Mass Spectrometer, in 47th ASMS Conference, Dallas, Tex. (1999), Loboda et al., Rapid Comm. Mass Spectrom. 14(12):1047-1057 (2000), Shevchenko et al., Anal. Chem., 72: 2132-2142 (2000), and Krutchinsky et al., J. Am. Soc. Mass Spectrom., 11(6):493-504 (2000).

7. Mass spectra are obtained from defined positions on the slide surface. The relative amount of each of the three peaks of multidimension signal polypeptides is used to determine the relative ratios of the antigens detected by the liposome-detector complexes.

The liposome carrier method is not limited to the detection of analytes on histological sections. Cells obtained by sorting may also be used for analysis in the disclosed method (Scheffold, A., Assenmacher, M., Reiners-Schramm, L., Lauster, R., and Radbruch, A., 2000, Nature Medicine 1 :107-110).

P. Labeled Proteins and Analytes

Labeled proteins are proteins or peptides to which one or more multidimension signals are attached. Preferably, the multidimension signal and the protein or peptide are covalently coupled or tethered to each other. Labeled analytes are analytes to which one or more multidimension signals are attached. Preferably, the multidimension signal and the analyte are covalently coupled or tethered to each other.

As used herein, molecules are coupled when they are covalent joined, directly or indirectly. The multidimension signal can be attached to the protein, peptide, or analyte in any manner. One non-limiting form of indirect coupling is via a linker molecule. The multidimension signal can be coupled to the protein, peptide, or analytes by any suitable coupling reactions. For example, multidimension signals can be covalently coupled to proteins or peptide through a sulfur-sulfur bond between a cysteine on the protein or peptide and a cysteine on the multidimension signal. Multidimension signals also can be attached to proteins and peptides by ligation (for example, protein ligation of a multidimension signal peptide to a protein). Many other chemistries and techniques for coupling compounds to proteins, peptides or analytes are known and can be used to couple multidimension signals to proteins, peptides, or analytes. For example, coupling can be made using thiols, epoxides, nitriles for thiols, NHS esters, isothiocyantes, isothiocyanates for amines, amines, and alcohols for carboxylic acids. Proteins, peptides, and analytes can also be labeled in vivo.

As used herein, “labeled protein” refers to both proteins and peptides to which one or more multidimension signals are attached. The term labeled protein refers both to proteins and peptides attached to intact (for example, unfragmented) multidimension signals and to proteins and peptides attached to modified (for example, fragmented) multidimension signals. The latter form of labeled proteins is referred to as fragmented labeled proteins. Although the protein portion of a labeled protein can be fragmented (for example, by protease digestion), the term fragmented labeled protein refers to a labeled protein where the multidimension signal has been fragmented. Isobaric labeled proteins are proteins or peptides of the same type that are labeled with isobaric multidimension signals such that a set of the proteins has the same mass-to-charge ratio.

As used herein, “labeled analyte” refers to analytes to which one or more multidimension signals are attached. The term labeled analyte refers both to analytes attached to intact (for example, unfragmented) multidimension signals and to analytes attached to modified (for example, fragmented) multidimension signals. The latter form of labeled proteins is referred to as fragmented labeled analytes. Although the analyte portion of a labeled analyte can be fragmented, the term fragmented labeled analyte refers to a labeled analyte where the multidimension signal has been fragmented. Isobaric labeled analytes are analytes of the same type that are labeled with isobaric multidimension signals such that a set of the analytes has the same mass-to-charge ratio.

A protein, peptide, or analyte sample to be analyzed can also be subjected to fractionation or separation to reduce the complexity of the samples. Fragmentation and fractionation can also be used together in the sme assay. Such fragmentation, fractinatnion, or separation can simplify and extend the analysis of proteins, peptides, and analytes.

In one non-limiting example, it is possible to form labeled proteins where the multidimension signal is specifically attached to phosphopeptides. Chemistry for specific derivatization of phosphoserine or phosphotyrosine residues has been described (Zhou et al. A systematic approach to the problem ofprotein phosphorylation., Nat. Biotech. 19:375-378 (2001); Oda et al., Enrichment analysis of phosphorylated proteins as a tool for probing the phosphoproteome., Nat. Biotech. 19:379-382 (2001)). Tryptic peptides treated according to either of these two protocols will display reactive sulfhydryls at sites of protein phosphorylation. These sites may be reacted with multidimension signals to generate a labeled protein. Non-phosphorylated peptides will not be derivatized.

Q. Affinity Tags

An affinity tag is any compound that can be used to separate compounds or complexes having the affinity tag from those that do not. Preferably, an affinity tag is a compound, such as a ligand or hapten, that associates or interacts with another compound, such as ligand-binding molecule or an antibody. It is also preferred that such interaction between the affinity tag and the capturing component be a specific interaction, such as between a hapten and an antibody or a ligand and a ligand-binding molecule. Affinity tags preferably are antibodies, ligands, binding proteins, receptor proteins, haptens, aptamers, carbohydrates, synthetic polyamides, or oligonucleotides. Preferred binding proteins are DNA binding proteins. Preferred DNA binding proteins are zinc finger motifs, leucine zipper motifs, helix-turn-helix motifs. These motifs can be combined in the same specific binding molecule.

Affinity tags, described in the context of nucleic acid probes, are described by Syvnen et al., Nucleic Acids Res., 14:5037 (1986). Preferred affinity tags include biotin, which can be incorporated into nucleic acids. In the disclosed method, affinity tags incorporated into multidimension signals can allow the multidimension signals to be captured by, adhered to, or coupled to a substrate. Such capture allows separation of multidimension signals from other molecules, simplified washing and handling of multidimension signals, and allows automation of all or part of the method.

Zinc fingers can also be used as affinity tags. Properties of zinc fingers, zinc finger motifs, and their interactions, are described by Nardelli et al., Zinc finger-DNA recognition: analysis of base specificity by site-directed mutagenesis. Nucleic Acids Res, 20(16):4137-44 (1992), Jamieson et al., In vitro selection of zinc fingers with altered DNA-binding specificity. Biochemistry, 33(19):5689-95 (1994), Chandrasegaran, S. and J. Smith, Chimeric restriction enzymes: what is next? Biol Chem, 380(7-8):841-8 (1999), and Smith et al., A detailed study of the substrate specificity of a chimeric restriction enzyme. Nucleic Acids Res, 27(2):674-81 (1999).

Capturing multidimension signals on a substrate, if desired, may be accomplished in several ways. In one embodiment, affinity docks are adhered or coupled to the substrate. Affinity docks are compounds or moieties that mediate adherence of a multidimension signal by associating or interacting with an affinity tag on the multidimension signal. Affinity docks immobilized on a substrate allow capture of the multidimension signals on the substrate. Such capture provides a convenient means of washing away molecules that might interfere with subsequent steps. Captured multidimension signals can also be released from the substrate. This can be accomplished by dissociating the affinity tag or by breaking a photocleavable linkage between the multidimension signal and the substrate.

Substrates for use in the disclosed method can include any solid material to which multidimension signals can be adhered or coupled. Examples of substrates include, but are not limited to, materials such as acrylamide, cellulose, nitrocellulose, glass, silicon, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates can have any useful form including thin films or membranes, beads, bottles, dishes, fibers, optical fibers, woven fibers, shaped polymers, particles, compact disks, and microparticles.

R. Vectors and Expression Sequences

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

As used herein, plasmid or viral vectors are agents that transport the gene into the cell without degradation and include a promoter yielding expression of the gene in the cells into which it is delivered. In a preferred embodiment vectors are derived from either a virus or a retrovirus. Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes; they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

1. Retroviral Vectors

A retrovirus is an animal virus belonging to the virus family of Retroviridae, including any types, subfamilies, genus, or tropisms. Retroviral vectors, in general, are described by Venna, I. M., Retroviral vectors for gene transfer. In Microbiology-1 985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

2. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the El gene removed and these virons are generated in a cell line such as the human 293 cell line. In another preferred embodiment both the El and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

3. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindlIl E restriction fragment (Greenway, P. J. et al., Gene 18: 355-360 (1982)). Of course, promoters from the host cell or related species also are useful herein.

Enhancer generally refers to a sequence of DNA that finctions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their finction. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region act as a constitutive promoter and/or enhancer to maximize expression of the region of the transcription unit to be transcribed. It is further preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

4. Markers

The viral vectors can include nucleic acid sequence encoding a marker product. This marker product is used to determine if the gene has been delivered to the cell and once delivered is being expressed. Preferred marker genes are the E. Coli lacZ gene which encodes β-galactosidase and green fluorescent protein. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

S. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for analysis of analytes, the kit comprising a set of reporter signals and one or more indicator signals.

T. Mixtures

Disclosed are mixtures formed by performing or preparing to perform the disclosed method. For example, disclosed are mixtures comprising multidimension signals, reporter signals, indicator signals, or a combination.

Whenever the method involves mixing or bringing into contact compositions or components or reagents, performing the method creates a number of different mixtures. For example, if the method includes 3 mixing steps, after each one of these steps a unique mixture is formed if the steps ate performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

U. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising a mass spectrometer with a means for analyzing patterns and selecting portions of analysis samples for further analysis.

V. Data Structures and Computer Control

Disclosed are data structures used in, generated by, or generated from, the disclosed method. Data structures generally are any form of data, information, and/or objects collected, organized, stored, and/or embodied in a composition or medium. A protein signature stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Illustrations

The disclosed methods can be further understood by way of the following illustrations which involve examples of the disclosed methods. The illustrations are not intended to limit the scope of the method in any way.

A. Illustration 1: Set of Isobaric Reporter Signals and an Indicator Signal; Heavy Isotopes

This illustration makes use of peptide reporter signals having the same mass, that fragment at certain peptide bonds, and that use heavy isotopes to distribute mass differently in different reporter signals. For example, it has been demonstrated, in ion traps, that peptides containing arginine will preferentially fragment at the C-termini of aspartic acid or glutamic acid residues, and, proline containing peptides will fragment at the N-termini of the proline residues (Qin and Chait, Int. J. Mass Spectrom. (Netherlands), 190-191:313-20 (1999)). DP (aspartic acid (D) and proline (P)) amino acid sequences can be used in the disclosed reporter signals resulting in collisionally induced fragmentation at the scissile bond between the aspartic acid and proline.

The singly charged ion of an exemplary peptide, AGSLDPAGSLR (SEQ ID NO:2), will fragment between the ‘D’ and ‘P’ in the collision cell of the mass spectrometer. Utilizing natural abundance isotopes the singly charged parent ion will have an average nominal (m/z)=1043 amu, and the possible resultant daughter ions AGSLD⁺ (amino acids 1-5 of SEQ ID NO:2) and PAGSLR⁺ (amino acids 6-11 of SEQ ID NO:2) have average nominal (m/z) of 461 and 600 amu, respectively. As a practical matter, fragmentation will typically yield one dominant daughter ion, say PAGSLR⁺ (amino acids 6-11 of SEQ ID NO:2) in this case. For this illustration consider only one charged daughter from the population of singly charged parent. Note that, without loss of generality or applicability, the branching ratio into these daughter ion channels may be other than 100% into the PAGSLR⁺ (amino acids 6-11 of SEQ ID NO:2) daughter fragment.

Standard synthetic methods can be utilized to construct such peptides. In this illustration of reporter molecules consider isotopically labeled amino acids (for example, A vs. A*, where A has a CH₃ and A* has a CD₃ side chain). There are four possibilities for the synthetic peptide, with their nominal (m/z) indicated in parentheses: AGSLDPAGSLR (1043), A*GSLDPAGSLR (1046), AGSLDPA*GSLR (1046), A*GSLDPA*GSLR (1049) (SEQ ID NO:2). For this example consider the two mono-labeled peptides A*GSLDPAGSLR, AGSLDPA*GSLR (SEQ ID NO:2), which have a common nominal mass-to-charge of 1046, as reporter signals and the unlabeled peptide AGSLDPAGSLR (SEQ ID NO:2), which has a nominal mass-to-charge of 1043, as an indicator signal.

As a simple demonstration of a preferred mode of the disclosed method consider a solution containing the three synthetic peptides. This solution could have been collected following any number of biological experiments and, in general, because of processing, would contain many additional components.

The solution containing AGSLDPAGSLR, A*GSLDPAGSLR and AGSLDPA*GSLR (SEQ ID NO:2) is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the tandem mass spectrometer of a quadrupole time of flight type (e.g. Applied Biosystems QSTAR or Waters QtoF). Utilizing the laser impinging on the sample spot on the MALDI target, many ions are introduced into the first quadrupole, Q0. Among the species introduced into Q0 are predominantly singly charged species (AGSLDPAGSLR⁺, A*GSLDPAGSLR⁺, AGSLDPA*GSLR⁺; SEQ ID NO:2), various fragmentation ions, neutral matrix, matrix ions and multimers as known in the art. Neutral particles will pass out of Q0 without being guided into the second quadrupole, Q1.

Ions introduced into Q0 are guided into the higher vacuum region containing Q1, which is operated in DC field only (acting as an ion pipe rather than a mass-to-charge filter), and detected on the time of flight analyzer. The resulting spectrum (MS Spectrum) is analyzed for a doublet peak separated by m/z=3. Based on the identification of doublet peaks, quadrupole Q1 is set to pass ions with the higher mass-to-charge ratio of the doublet into the third quadrupole, Q2 (recall A*GSLDPAGSLR and AGSLDPA*GSLR (SEQ ID NO:2) have the same mass-to-charge; “isobaric” in the parlance of mass spectrometry). Ions with mass-to-charge ratios different from 1046 will follow trajectories that do not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause preferential cleavage of the DP scissile bond of the peptide ions, typically a few milliTorr of Argon or Nitrogen. As discussed above, the fragmentation of the singly charged parent ion is expected to yield predominantly one daughter ion. In this case each of the isobaric parents (SEQ ID NO:2) will yield correlated, unique daughters (amino acids 1-5 and 6-11 of SEQ ID NO:2):
A*GSLDPAGSLR⁺→A*GSLD+PAGSLR⁺(m/z 600)
AGSLDPA*GSLR⁺→AGSLD+PA*GSLR⁺(m/z 603)

The resolution of the mass spectrometers as discussed here is on the order of 5000 to 10000, and thus the 3 amu difference is readily attained at these (m/z).

The ions exiting Q2 enter the time-of-flight (TOF) section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than lighter ions.

The resulting mass spectrum (MS/MS spectrum) reflects the relative amount of the two analytes (for example, peptides) in the original sample.

The advantage of the identification of the predetermined pattern (doublet peaks separated by m/z=3) and subsequent passing of the peak with the higher m/z in the doublet is more apparent in assays involving more multidimension signals of a variety of m/z. In such a case the MS spectrum can be analyzed for doublets and only peaks involved in the predetermined pattern will be passed on for collection of MS/MS spectra. This scheme can be extended to more analytes (for example, peptides). The most basic extension for a panel of isobaric detectors based upon the above peptide, utilizing X/X* differences, would be as shown in Table 2. The asterisk indicates heavy isotope labeled amino acids. This set assumes that the non-labeled to labeled mass change {(m/z)ₓ*−(m/z)ₓ} for each residue is the same. For the general case where {(m/z)ₓ*−(m/z)ₓ} is not the same for all the residues there are more combinations for a given peptide which can be resolved by the mass spectrometer. The parent molecule is SEQ ID NO:2 and the primary daughter is amino acids 6-11 of SEQ ID NO:2.

TABLE 2

| | |
|---|---|
| Parent | Primary Daughter |

| | |
|---|---|
| A*G*S*L*DPAGSLR | PAGSLR |
| AG*S*L*DPA*GSLR | PA*GSLR |
| AGS*L*DPA*G*SLR | PA*G*SLR |
| AGSL*DPA*G*S*LR | PA*G*S*LR |
| AGSLDPA*G*S*L*R | PA*G*S*L*R |

The synthesis of specific isotope labeled amino acids would facilitate rapidly increased panel size. For example, synthesis of unique alanines with CH₃, CH₂D, CHD₂, CD₃ side chains could be used to yield a significant panel size with a small peptide.

This mode of the disclosed method has the desirable property that all the detected ions originate from a very similar chemical environment (only differing by the location of a few neutrons) and will thus behave identically (for all practical purposes) when processed in the MALDI source and in the collision cell. Of particular note is the case where one of the isobaric reporter signal molecules is added as a quantitation standard to the isobaric detector molecules used for the assay. Quantitation of the entire set of detector molecules used in the assay is straightforward and quantitative. For the case where the molecules are essentially identical except for the isotopic enrichment all the isobars in a set will behave identically through the processing.

B. Illustration 2: Two Isobaric Sets of Multidimension Signals; Scissile Bond.

This illustration makes use of peptide reporter signals having the same mass that fragment at certain peptide bonds, where the bond is placed in different locations in the different reporter signals. As discussed above, DP containing amino acid sequence will fragment between the aspartic acid and proline in a collision cell. Sets of peptides that can be useful for the disclosed method can be:

| | |
|---|---|
| Isobaric Set 1: | |
| Peptide C: YFMTSGCDPGGR | (SEQ ID NO:13) |
| Peptide D: YFMTSGDPCGGR | (SEQ ID NO:14) |
| Peptide E: YFMTSDPGCGGR | (SEQ ID NO:15) |
| Peptide F: YFMTDPSGCGGR | (SEQ ID NO:16) |
| Peptide G: YFMDPTSGCGGR | (SEQ ID NO:17) |
| Isobaric Set 2: | |
| Peptide H: YFMTSGCDPGAR | (SEQ ID NO:18) |
| Peptide I: YFMTSGDPCGAR | (SEQ ID NO:19) |
| Peptide J: YFMTSDPGCGAR | (SEQ ID NO:20) |
| Peptide K: YFMTDPSGCGAR | (SEQ ID NO:21) |
| Peptide L: YFMDPTSGCGAR | (SEQ ID NO:22) |

The peptides in the two sets differ in the position of the DP dipeptide and in the amino acid at position 11 (glycine or alanine). The peptides in Isobaric Set 1 differ in mass from the peptides of Isobaric Set 2 by 14 amu (based on the mass difference between gylcine and alanine).

For simplicity consider a solution containing these synthetic peptides. This solution could have been collected following any number of biological experiments and, in general, because of processing would contain many additional components.

The solution containing C, D, E, F, G, H, I, J, K, L is mixed with a suitable matrix solution for performing analysis by mass spectrometry. Suitable matrices, including sinapic acid, 4-hydroxy-α-cyanocinamic acid or 2,5-dihydroxybenzoic acid, are known in the art.

The resulting solution is spotted onto the MALDI target and allowed to crystallize.

The target is inserted into the source of the tandem mass spectrometer of a quadrupole time of flight type (e.g. Applied Biosystems QSTAR or Waters QtoF).

Utilizing the laser impinging on the spot on the MALDI target, many ions are introduced into the first quadrupole, Q0. Among the species introduced into Q0 are C⁺, D⁺, E⁺, F⁺, G⁺, H⁺, I⁺, J⁺, K⁺, L⁺, various fragmentation ions, matrix ions and multimers as known in the art. Neutral particles will pass out of QO without being guided into Q1.

Ions introduced into Q0 are guided into the higher vacuum region containing Q1, which is operated in DC field only (acting as an ion pipe rather than a mass-to-charge filter), and detected on the time of flight analyzer. The resulting spectrum (MS Spectrum) is analyzed for a doublet peak separated by m/z=14. Based on the identification of doublet peaks, quadrupole Q1 is set to pass separately ions with the lower mass-to-charge ratio of the doublet ((m/z)_{C}, (m/z)_{D}, (m/z)_{E}, (m/z)_{F}, (m/z)_{G}; they have the same molecular weight “isobaric”) and ions with the higher mass-to-charge ratio of the doublet ((m/z)_{H}, (m/z)_{I}, (m/z)_{J}, (m/z)_{K}, (m/z)_{L}; they have the same molecular weight “isobaric”). Ions with mass-to-charge ratios different from (m/z)_{C}, (m/z)_{D}, (m/z)_{E}, (m/z)_{F}, (m/z)_{G}, (m/z)_{H}, (m/z)_{I}, (m/z)_{J}, (m/z)_{K}, (m/z)_{L} will follow trajectories which will not exit Q1 on the Q1-Q2 axis, and are effectively discarded. This yields a huge increase in the signal to noise for the system, on the order of 100-1000 fold improvement over systems which do not have this mass filtering.

The collision cell surrounding Q2 is filled with a chemically inert gas at an appropriate pressure to cause scission of the D-P bond, typically a few milliTorr of Argon or Nitrogen. Considering only ions with the lower mass-to-charge ratio of the doublet, fragmentation at the DP bond, total retention of the charge by the C termini fragments, and the operation of Q2 in RF only mode, there will be five possible ions which can emerge from Q2 into the TOF section.

| | |
|---|---|
| C1^{+: PGGR}⁺ | (amino acids 9-12 of SEQ ID NO:13) |
| D1^{+: PCGGR}⁺ | (amino acids 8-12 of SEQ ID NO:14) |
| E1^{+: PGCGGR}⁺ | (amino acids 7-12 of SEQ ID NO:15) |
| F1^{+: PSGCGGR}⁺ | (amino acids 6-12 of SEQ ID NO:16) |
| G1^{+: PTSGCGGR}⁺ | (amino acids 5-12 of SEQ ID NO:17) |

A similar series of fragmentation ions will result from Q2 analysis of the ions with the higher mass-to-charge ratio of the doublet

The ions exiting Q2 enter the time-of-flight, TOF, section of the instrument. A transient electric field gradient is applied and the positively charged ions are accelerated toward the reflectron and ultimately to the detector. The ions are all accelerated through the same electric field gradient (the reflectron will compensate for a small perturbation in this assertion, as is known in the art) and thus will all have the same kinetic energy imparted to them. Because the kinetic energy is the same for all ions, and the masses of the ions are different, the time it takes for the ions to reach the detector will be different: heavier ions will arrive later than light ions.

The resulting mass spectrum (MS/MS spectrum) will indicate the relative amount of the analytes (for example, peptides) in the original sample.

The advantage of the identification of the predetermined pattern (doublet peaks separated by m/z=14) and subsequent passing of the peaks of the doublet is more apparent in assays involving more multidimension signals of a variety of m/z. In such a case the MS spectrum can be analyzed for doublets and only peaks involved in the predetermined pattern will be passed on for collection of MS/MS spectra.

A standard with the same mass as the analytes could have been added to facilitate quantitative results. In order to extract quantitative results the relative efficiencies of molecules under consideration should be determined to be used in calibration; a straightforward process.

### EXAMPLES

This example provides an example of the disclosed methods involving labeling of proteins with multidimension signals and pattern recognition in the MS dimension for collection and analysis of MS/MS data.

Consider a two-sample assay as shown in FIG. 1. In this assay, bovine serum albumin (BSA) was chosen as an exemplary protein. A common BSA sample was split into two parts (constituting the two samples), and reacted with sets of multidimension signals (Table 3).

Two sets of multidimension labels were used (Label Set 1 and Label Set 2; see Table 3). The members of a given set are isobaric (all the members of Label Set 1 are isobaric to each other and all the members of Label Set 2 are isobaric to each other). That is, within the sets the labels are isobaric. Such sets can be referred to as isobaric sets. The members of Label Set 1 are not isobaric to the member of Label Set 2. That is, Label Set 1 and Label Set 2 are not isobaric to each other. The specifics of the multidimension signals are shown in Table 3.

TABLE 3

Selected attributes of multidimension signals
(labels) for labeling cysteine side chains.

| | |
|---|---|
| Label Set 1, Member 1 | Rx-GGGGGGdpgggggg |
| Label Set 1, Member 2 | Rx-GGGGGgdpGggggg |
| Label Set 1, Member 3 | Rx-GGGGggdpGGgggg |
| Label Set 1, Member 4 | Rx-GGGgggdpGGGggg |
| Label Set 1, Member 5 | Rx-GGggggdpGGGGgg |
| Label Set 1, Member 6 | Rx-GgggggdpGGGGGg |
| Label Set 1, Member 7 | Rx-ggggggdpGGGGGG |
| Label Set 2, Member 1 | Rx-ggggdpgggggggg |
| Label Set 2, Member 2 | Rx-gggggdpggggggg |
| Label Set 2, Member 3 | Rx-ggggggdpgggggg |
| Label Set 2, Member 4 | Rx-gggggggdpggggg |
| Label Set 2, Member 5 | Rx-ggggggggdpgggg |

Rx represents a sulfhydryl reactive group (including a short linker) which generates a covalent attachment by alkylation. g represents a glycine residue; G represents a glycine residue which has been enriched in ¹³C (2 places) and ¹⁵N (1 place) relative to g. Note that members of Label Set 1 are nominally 18 Daltons heavier than members of Label Set 2, due to the incorporation of 6 heavy glycine residues.

Bovine serum albumin (BSA, Sigma Cat# A7030) was dissolved in denaturation buffer (50 mM ammonium bicarbonate, pH 8.5, 6 M Urea, 0.5 mM Tris(2-carboxyethyl)phosphine hydrochloride or TCEP) and denatured by incubating at 37° C. for 30 minutes. iPROT peptide labels were synthesized and purified by American Peptide Co. Each label was dissolved in DMSO to 10 mg/ml concentration. Nominally equimolar cocktails of isobaric iPROT labels were prepared by combining the same volumes of an isobaric set of labels. Two cocktails were produced, one with seven “heavy” labels (Label Set 1, Table 3), and one with five “light” labels (Label Set 2, Table 3). After denaturation, BSA was labeled by mixing 6 μg of label (either “heavy” or “light” cocktail) per 1 μg of BSA and incubating at room temperature (24-25° C.) for 2 hours in the dark. The iPROT concentration per labeling reaction was 3.6 mM. After labeling, β-mercaptoethanol was added to a final concentration of 80 mM to quench the excess label.

A mixture of non-isobaric sets of labeled BSA was then produced by mixing equal volumes of the “heavy” and “light” labeling reactions (see FIG. 1). The mixture of labeling solutions was then dialyzed against 0.1 M ammonium bicarbonate. Labeled BSA was digested with Trypsin immobilized to agarose beads (PIERCE Cat # 20230). First, beads were thoroughly rinsed in 0.1 M ammonium bicarbonate and prepared as a 50% slurry. One volume of this slurry was mixed with one volume of dialyzed BSA solution and incubated at 37° C. with agitation overnight (˜16 hours). The supernatant was recovered containing the iPROT-labeled tryptic peptides.

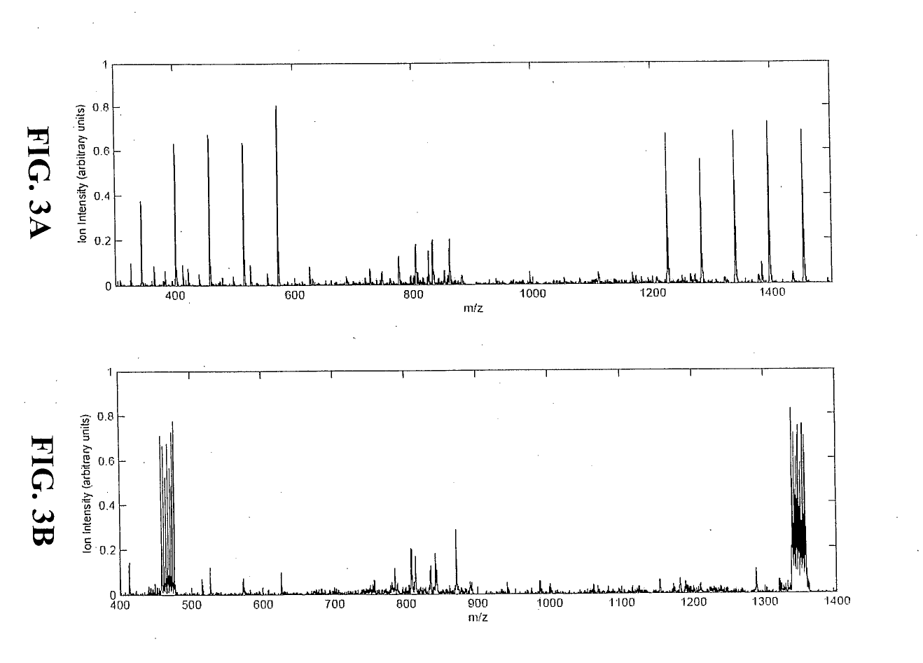

The resulting mixture was analyzed by LC/MS and LC/MS/MS. The sample peptides (representing trypsin fragments of BSA labeled with the multidimension signals) were separated according to their hydrophobicity by reverse phase high performance liquid chromatography as known in the art. Data were collected using an Agilient 1100 LC connected to Thermo Electron Corporation LTQ, or Applied Biosystems/MDS Sciex QSTAR Pulsar with o-MALDI source. The resulting fractions were analyzed by MALDI tandem mass spectrometry and by ESI tandem mass spectrometry. Exemplary spectra of the LC run are show in FIGS. 2A and 2B are graphs of mass spectrometry spectra of bovine serum albumin fragments labeled with multidimension signals. FIG. 2A covers m/z 1200 to 2500. FIG. 2B covers m/z from 500 to 1200. These spectra represent an example of an indicator level of analysis in the disclosed methods in which predetermined patterns are to be identified., the MALDI data (FIG. 2A) dominated by singly charged species (i.e. z=1) and ESI (FIG. 2B) dominated by multiply charged species (z=2.3).

The patterns of the pairs of ions are quite recognizable, and represent several ionic species. FIG. 2A covers m/z 1200 to 2500. FIG. 2B covers m/z from 500 to 1200. These spectra represent an example of an indicator level of analysis in the disclosed methods in which predetermined patterns are to be identified. FIG. 2A is from MALDI QSTAR instrument. The doublets spaced by 18 Dalton correspond to the mass difference between members of Label Set 1 (heavy) and Label Set 2 (light) shown in Table 3. The pair near m/z =1360 are spaced apart by 36 Dalton, corresponding to a peptide with two cysteines and thus two multidimension signals. The presence of two multidimension signals doubles the mass difference between the fragment labeled with a member of Label Set 1 and a member of Label Set 2. FIG. 2B is from ESI LTQ FTMS. The doublets are spaced apart by 18 Dalton correspond to the mass difference between members of Label Set 1 (heavy) and Label Set 2 (light) shown in Table 3. These doublets (spaced at multiples of 18 Daltons) represent a predetermined pattern expected from the use of multidimension labels in Label Set 1 and Label Set 2. These individual ionic species can be extracted by conducting MS/MS experiments.

Exemplary MS/MS spectra from the ESI LTQ FTMS instrument are shown in FIGS. 3A and 3B are graphs of mass spectrometry spectra of bovine serum albumin fragments labeled with multidimension signals. These spectra represent an example of a reporter level of analysis in the disclosed methods in which portions of a sample identified by predetermined patterns are subjected to further analysis (MS/MS in this case). FIG. 3A is a MS/MS spectrum of the peak at m/z 898.44 shown in FIG. 2B (lighter peak of the doublet). This peak represents a portion of the sample analyzed in FIG. 2B identified for the further analysis shown in FIG. 3A based on a predetermined pattern (peak doublets spaced at multiples of 18 Daltons). This peak represents protein fragments labeled with multidimension signals from Label Set 2 (the lighter set; see Table 3). The multidimension signals fragment at the D-P residues in the signals to produce pairs of fragments of characteristic mass. The two sets of 5 peaks in FIG. 3A represent pairs of fragments that result from fragmentation of the multidimension signals (one peak from one set of peaks paired with a peak from the other set). The peaks in a set of 5 peaks are separated by about 60 Daltons. The spectra of FIGS. 3A and 3B are graphs of mass spectrometry spectra of bovine serum albumin fragments labeled with multidimension signals. These spectra represent an example of a reporter level of analysis in the disclosed methods in which portions of a sample identified by predetermined patterns are subjected to further analysis (MS/MS in this case). FIG. 3A is a MS/MS spectrum of the peak at m/z 898.44 shown in FIG. 2B (lighter peak of the doublet). This peak represents a portion of the sample analyzed in FIG. 2B identified for the further analysis shown in FIG. 3A based on a predetermined pattern (peak doublets spaced at multiples of 18 Daltons). This peak represents protein fragments labeled with multidimension signals from Label Set 2 (the lighter set; see Table 3). The multidimension signals fragment at the D-P residues in the signals to produce pairs of fragments of characteristic mass. The two sets of 5 peaks in FIG. 3A represent pairs of fragments that result from fragmentation of the multidimension signals (one peak from one set of peaks paired with a peak from the other set). The peaks in a set of 5 peaks are separated by about 60 Daltons correspond to the selection of the double charge state ion near m/z=900 seen in FIGS. 2A and 2B are graphs of mass spectrometry spectra of bovine serum albumin fragments labeled with multidimension signals. FIG. 2A covers m/z 1200 to 2500. FIG. 2B covers m/z from 500 to 1200. These spectra represent an example of an indicator level of analysis in the disclosed methods in which predetermined patterns are to be identified. B (one peak of the doublet analyzed in FIG. 3A and the other analyzed in FIG. 3B), followed by collisionally induced fragmentation yielding two singly charged fragments (one group centered near m/z=460, the other centered near m/z=1350). FIG. 3A is a MS/MS spectrum of the peak at m/z 898.44 shown in FIG. 2B (lighter peak of the doublet). This peak represents a portion of the sample analyzed in FIG. 2B identified for the further analysis shown in FIG. 3A based on a predetermined pattern (peak doublets spaced at multiples of 18 Daltons). This peak represents protein fragments labeled with multidimension signals from Label Set 2 (the lighter set; see Table 3). The multidimension signals fragment at the D-P residues in the signals to produce pairs of fragments of characteristic mass. The two sets of 5 peaks in FIG. 3A represent pairs of fragments that result from fragmentation of the multidimension signals (one peak from one set of peaks paired with a peak from the other set). The peaks in a set of 5 peaks are separated by about 60 Daltons.

FIG. 3B is a MS/MS spectrum of the peak at m/z 907.45 shown in FIG. 2B (heavier peak of the doublet). This peak represents a portion of the sample analyzed in FIG. 2B identified for the further analysis shown in FIG. 3B based on a predetermined pattern (peak doublets spaced at multiples of 18 Daltons). This peak represents protein fragments labeled with multidimension signals from Label Set 1 (the heavier set; see Table 3). The multidimension signals fragment at the D-P residues in the signals to produce pairs of fragments of characteristic mass. The two sets of 7 peaks in FIG. 3B (which are tightly spaced in the graph) represent pairs of fragments that result from fragmentation of the multidimension signals (one peak from one set of peaks paired with a peak from the other set). The peaks in a set of 7 peaks are separated by about 3 Daltons (which is not well resolved at the resolution of the graph).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a reporter signal” includes a plurality of such reporter signals, reference to “the oligonucleotide” is a reference to one or more oligonucleotides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1-33. (canceled)

34. A reporter signal peptide comprising the amino acid sequence Gly-Gly-Gly-Gly-Gly-Gly-Asp-Pro-Gly-Gly-Gly-Gly-Gly-Gly, wherein six of the Gly residues have a common molecular mass that is different from a common molecular mass of the remaining six of the Gly residues.

35. The reporter signal peptide of claim 34, wherein common molecular mass of six of the Gly residues differs from the common molecular mass of the remaining six of the Gly residues by isotopic enrichment of one or more of the atoms of the Gly residues.

36. The reporter signal peptide of claim 35, wherein the isotopically enriched Gly residues include one or more ¹³C atoms.

37. The reporter signal peptide of claim 35, wherein the isotopically enriched Gly residues include an ¹⁵N atom.

38. The reporter signal peptide of claim 35, wherein the isotopically enriched Gly residues include two ¹³C atoms and one ¹⁵N atom.

39. The reporter signal peptide of claim 34, wherein the reporter signal peptide can be fragmented across the Asp-Pro peptide bond by collision-induced dissociation in an ion trap mass spectrometer.

40. The reporter signal peptide of claim 34, further comprising a coupling agent for covalent coupling to a protein or a peptide.

41. The reporter signal peptide of claim 40, wherein the coupling agent comprises a chemically reactive group.

42. The reporter signal peptide of claim 41, wherein the coupling agent further comprises a linker linking the chemically reactive group to the reporter signal peptide.

43. The reporter signal peptide of claim 41, wherein the chemically reactive group can covalently couple with a free sulfhydryl group of a cysteine residue.

44. The reporter signal peptide of claim 43, wherein the chemically reactive group is selected from the group consisting of thiols, epoxides, or nitriles.

45. The reporter signal peptide of claim 43, wherein the chemically reactive group is an alpha-haloacetyl.

46. The reporter signal peptide of claim 43, wherein the chemically reactive group is an iodoacetyl or an iodoacetamide.

47. The reporter signal peptide of claim 41, wherein the chemically reactive group can react with a free amino-terminal primary amino group of a protein or a peptide.

48. The reporter signal peptide of claim 47, wherein the chemically reactive group is selected from the group consisting of an NHS ester, an isothiocyanate, and an acetylating agent.

49. The reporter signal peptide of claim 48, wherein the chemically reactive group is an NHS ester.

50. A reporter signal peptide of claim 34 selected from the group consisting of: GGGGGGDPgggggg; GGGGGgDPGggggg, GGGGggDPGGgggg, GGGgggDPGGGggg, GGggggDPGGGGgg, GgggggDPGGGGGg, and ggggggDPGGGGGG, wherein “G” Gly residues have a higher molecular mass than “g” Gly residues.

51. A reporter signal peptide of claim 50, wherein the “G” Gly residues comprise two ¹³C atoms and one ¹⁵N atom.

52. A reporter signal peptide of claim 50, further comprising a chemically reactive group.

53. A reporter signal peptide of claim 52, wherein the chemically reactive group is selected from the group consisting of: a thiol, an epoxide, a nitrile, an NHS ester, an isothiocyanate, and an acetylating agent.

54. A set of reporter signal peptides comprising two or more reporter signal peptides of claim 34, wherein each of the reporter signal peptides has the same molecular mass.

55. The set of reporter signal peptides of claim 54, wherein each of the reporter signal peptides has the same mass-to-charge ratio following ionization in a mass spectrometer.

56. The set of reporter signal peptides of claim 54, wherein each of the reporter signal peptides can be fragmented across the Asp-Pro peptide bond by collision-induced dissociation in an ion trap mass spectrometer.

57. The set of reporter signal peptides of claim 56, wherein the mass-to-charge ratio of each fragmented reporter signal peptide in the set can be distinguished from the mass-to-charge ratio of the other fragmented reporter signal peptides in the set.

58. The set of reporter signal peptides of claim 57, wherein the reporter signal peptides further comprise a coupling agent having a chemically reactive group for covalent coupling to a target protein or peptide.

59. The set of reporter signal peptides of claim 58, wherein the chemically reactive group covalently couples a free sulfhydryl group of the target protein or peptide.

60. The set of reporter signal peptides of claim 59, wherein the chemically reactive group is selected from the group consisting of: a thiol, an epoxide, and a nitrile.

61. The set of reporter signal peptides of claim 58, wherein the chemically reactive group covalently couples an amino-terminal primary amine group of the target protein or peptide.

62. The set of reporter signal peptides of claim 61, wherein the chemically reactive group is selected from the group consisting of: an NHS ester, an isothiocyanate, and an acetylating agent.

63. The set of reporter peptides of claim 58 comprising:
Rx-GGGGGGDPgggggg,
Rx-GGGGGgDPGggggg,
Rx-GGGGggDPGGgggg,
Rx-GGGgggDPGGGggg,
Rx-GGggggDPGGGGgg,
Rx-GgggggDPGGGGGg,
and
Rx-GGGGGGDPgggggg,
wherein Rx is the coupling agent, and G and g are Gly residues with, respectively, higher and lower molecular masses by isotopic enrichment of one or more of the atoms of the Gly residues.

64. The set of reporter peptides of claim 63, wherein the isotopically enriched Gly residues include one or more ¹³C atoms.

65. The set of reporter peptides of claim 63, wherein the isotopically enriched Gly residues include an ¹⁵N atom.

66. The set of reporter peptides of claim 63, wherein the isotopically enriched Gly residues include two ¹³C atoms and one ¹⁵N atom.

67. A method comprising:
labeling a protein or a peptide in a sample with a reporter signal peptide comprising the amino acid sequence Gly-Gly-Gly-Gly-Gly-Gly-Asp-Pro-Gly-Gly-Gly-Gly-Gly-Gly, wherein six of the Gly residues have a common molecular mass that is different from a common molecular mass of the remaining six of the Gly residues;
separating the labeled protein or peptide or fragments thereof from molecules having a different mass-to-charge ratio in a mass spectrometer;
fragmenting the reporter signal peptide by collision induced dissociation in an ion trap mass spectrometer; and
detecting fragmented reporter signal peptide.

68. The method of claim 67, further comprising quantifying the amount of the fragmented reporter signal peptide.

69. The method of claim 68, further comprising comparing the amount of the fragmented reporter signal peptide to a known or an expected value.

70. The method of claim 67, further comprising denaturing the protein or peptide prior to labeling it with the reporter signal peptide.

71. The method of claim 67, further comprising producing the sample by a separation procedure.

72. The method of claim 71, wherein the separation procedure is selected from the group consisting of liquid chromatography, gel electrophoresis, two-dimensional chromatography, two-dimensional gel electrophoresis, isoelectric focusing, thin layer chromatography, centrifugation, filtration, ion chromatography, immunoaffinity chromatography, membrane separation, and a combination thereof.

73. The method of claim 67, further comprising fragmenting the labeled protein or peptide before separating the labeled protein or peptide or fragments thereof in a mass spectrometer.

74. The method of claim 73, wherein the labeled protein or peptide is fragmented by digestion with a protease.

75. The method of claim 74, wherein the protease is trypsin.

76. A method comprising:
labeling a set of proteins or peptides in a sample with a set of reporter signal peptides of claim 54;
separating the set of labeled proteins or peptides or fragments thereof from molecules having a different mass-to-charge ratio in a mass spectrometer;
fragmenting the reporter signal peptides by collision induced dissociation in an ion trap mass spectrometer; and
detecting fragmented reporter signals; and
distinguishing the fragmented reporter signal peptides from each other.

77. The method of claim 76, further comprising quantifying the amount of a first fragmented reporter signal peptide.

78. The method of claim 77, further comprising quantifying the amount of a second fragmented reporter signal peptide.

79. The method of claim 78, further comprising comparing the amounts of the first and the second fragmented reporter signal peptides.

80. The method of claim 76, wherein the sample is a complex sample comprising multiple proteins.

81. The method of claim 76, further comprising producing the sample by a separation procedure.

82. The method of claim 81, wherein the separation procedure is selected from the group consisting of liquid chromatography, gel electrophoresis, two-dimensional chromatography, two-dimensional gel electrophoresis, isoelectric focusing, thin layer chromatography, centrifugation, filtration, ion chromatography, immunoaffinity chromatography, membrane separation, and a combination thereof.

83. The method of claim 76, further comprising denaturing the set of proteins or peptides prior to labeling them with the set of reporter signals.

84. The method of claim 76, further comprising fragmenting the labeled proteins or peptides before separating the set of labeled proteins or peptides or fragments thereof in a mass spectrometer.

85. The method of claim 76, wherein the labeled proteins or peptides are fragmented by digestion with a protease.

86. The method of claim 85, wherein the protease is trypsin.

87. A kit comprising:
a set of reporter signal peptides of claim 54; and
a set of instructions for use.

88. The kit of claim 87, further comprising at least one target peptide labeled with a reporter signal peptide of claim 34.

89. The kit of claim 88, wherein the protein or peptide comprises a cysteine amino acid residue.

90. A protein or peptide labeled with a reporter signal peptide of claim 34.

91. The labeled protein or peptide of claim 90, wherein the protein or peptide comprises a cysteine amino acid residue.

92. A set of proteins or peptides according to claim 90.

93. A set of labeled peptides or proteins labeled with a set of reporter signal peptides of claim 54.

94. A reporter signal peptide comprising a single Asn-Pro amino acid sequence, wherein the reporter signal peptide can be fragmented across the Asn-Pro peptide bond by chemical cleavage.

95. The reporter signal peptide of claim 94, wherein the peptide is from about 11 to about 35 amino acids in length.

96. The reporter signal peptide of claim 94, wherein the Asn-Pro peptide bond is chemically cleavable by contact with ammonia vapor or solution.

97. A reporter signal peptide comprising a single Glu-Pro amino acid sequence, wherein the reporter signal peptide can be fragmented across the Glu-Pro peptide bond by collision-induced dissociation in an ion trap mass spectrometer.

98. The reporter signal peptide of claim 97, wherein the peptide is from about 11 to about 35 amino acids in length.

99. The reporter signal peptide of claim 94, wherein the peptide comprises one or more isotopically enriched amino acids.

100. The reporter signal peptide of claim 99, wherein the one or more isotopically enriched amino acids comprises an isotope selected from the group consisting of ²H, ³H, ¹³C, ¹⁴C, ¹⁵N, ¹⁷O, ¹⁸O and combinations thereof.

101. The reporter signal peptide of claim 99, further comprising a coupling agent for covalent coupling to a protein or a peptide.

102. The reporter signal peptide of claim 101, wherein the coupling agent comprises a chemically reactive group.

103. The reporter signal peptide of claim 102, wherein the coupling agent further comprises a linker linking the chemically reactive group to the reporter signal peptide.

104. The reporter signal peptide of claim 101, wherein the chemically reactive group can covalently couple with a free sulfhydryl group of a cysteine residue.

105. The reporter signal peptide of claim 104, wherein the chemically reactive group is selected from the group consisting of thiols, epoxides, or nitrites.

106. The reporter signal peptide of claim 104, wherein the chemically reactive group is an alpha-haloacetyl.

107. The reporter signal peptide of claim 104, wherein the chemically reactive group is an iodoacetyl or an iodoacetamide.

108. The reporter signal peptide of claim 102, wherein the chemically reactive group can react with a free amino-terminal primary amino group of a protein or a peptide.

109. The reporter signal peptide of claim 108, wherein the chemically reactive group is selected from the group consisting of an NHS ester, an isothiocyanate, and an acetylating agent.

110. The reporter signal peptide of claim 109, wherein the chemically reactive group is an NHS ester.


SEQUENCE LISTING (22 sequences)

SEQ ID NO: 1 - protein, 12 aa, Artificial Sequence
  Description of Artificial Sequence; Note=synthetic construct
CGGGGDPGGGGR

SEQ ID NO: 2 - protein, 33 aa, Artificial Sequence
  Description of Artificial Sequence; Note=synthetic construct
AGSLDPAGSLRAGSLDPAGSLRAGSLDPAGSLR

SEQ ID NO: 3 - protein, 39 aa, Artificial Sequence
  Description of Artificial Sequence; Note=synthetic construct
AGSMLDPAGSMLRAGSMLDPAGSMLRAGSMLDPAGSMLR

SEQ ID NO: 4 - protein, 11 aa, Artificial Sequence
  Description of Artificial Sequence; Note=synthetic construct
AGSLADPGSLR

SEQ ID NO: 5 - protein, 11 aa, Artificial Sequence
  Description of Artificial Sequence; Note=synthetic construct
ALSLADPGSGR

SEQ ID NO: 6 - protein, 11 aa, Artificial Sequence
  Description of Artificial Sequence; Note=synthetic construct
ALSLGDPASGR

SEQ ID NO: 7 - protein, 11 aa, Artificial Sequence
  Description of Artificial Sequence; Note=synthetic construct
AGSDPLAGSLR

SEQ ID NO: 8 - protein, 11 aa, Artificial Sequence
  Description of Artificial Sequence; Note=synthetic construct
ADPGSLAGSLR

SEQ ID NO: 9 - protein, 15 aa, Artificial Sequence
  Description of Artificial Sequence; Note=synthetic construct
  VARIANT 2: Xaa is Phe (2-NO(2))
  VARIANT 3-7,10-14: Xaa can be any amino acid
CXXXXXXDPXXXXXR

SEQ ID NO: 10 - protein, 35 aa, Artificial Sequence
  Description of Artificial Sequence; Note=synthetic construct
  VARIANT 1-9,12-21,24-30,32-35: Xaa can be any amino acid
XXXXXXXXXDPXXXXXXXXXXDPXXXXXXXRXXXX

SEQ ID NO: 11 - protein, 34 aa, Artificial Sequence
  Description of Artificial Sequence; Note=synthetic construct
  VARIANT 22: Xaa is Phe(2-NO(2))
  VARIANT 1-9,12-21,23-29,31-34: Xaa can be any amino acid
XXXXXXXXXDPXXXXXXXXXXXXXXXXXXRXXXX

SEQ ID NO: 12 - protein, 15 aa, Artificial Sequence
  Description of Artificial Sequence; Note=synthetic construct
  VARIANT 2: Xaa is Phe (2-NO(2))
  VARIANT 3-14: Xaa can be any amino acid
CXXXXXXXXXXXXXR

SEQ ID NO: 13 - protein, 12 aa, Artificial Sequence
  Description of Artificial Sequence; Note=synthetic construct
YFMTSGCDPGGR

SEQ ID NO: 14 - protein, 12 aa, Artificial Sequence
  Description of Artificial Sequence; Note=synthetic construct
YFMTSGDPCGGR

SEQ ID NO: 15 - protein, 12 aa, Artificial Sequence
  Description of Artificial Sequence; Note=synthetic construct
YFMTSDPGCGGR

SEQ ID NO: 16 - protein, 12 aa, Artificial Sequence
  Description of Artificial Sequence; Note=synthetic construct
YFMTDPSGCGGR

SEQ ID NO: 17 - protein, 12 aa, Artificial Sequence
  Description of Artificial Sequence; Note=synthetic construct
YFMDPTSGCGGR

SEQ ID NO: 18 - protein, 12 aa, Artificial Sequence
  Description of Artificial Sequence; Note=synthetic construct
YFMTSGCDPGAR

SEQ ID NO: 19 - protein, 12 aa, Artificial Sequence
  Description of Artificial Sequence; Note=synthetic construct
YFMTSGDPCGAR

SEQ ID NO: 20 - protein, 12 aa, Artificial Sequence
  Description of Artificial Sequence; Note=synthetic construct
YFMTSDPGCGAR

SEQ ID NO: 21 - protein, 12 aa, Artificial Sequence
  Description of Artificial Sequence; Note=synthetic construct
YFMTDPSGCGAR

SEQ ID NO: 22 - protein, 12 aa, Artificial Sequence
  Description of Artificial Sequence; Note=synthetic construct
YFMDPTSGCGAR